(19) United States
(12) Patent Application Publication
(10) Pub. No.: US 2026/0325729 A1
(43) Pub. Date: Oct. 22, 2026

(54) System and Method for Gene and/or Cellular Therapy

(71) Applicants: Innovative Cellular Therapeutics Holdings, Ltd., George Town (KY); Innovative Cellular Therapeutics, Inc., Rockville, MD (US)

(72) Inventors: Lei Xiao, Rockville, MD (US); Zhiyuan Cao, Rockville, MD (US); Zhao Wu, Shanghai (CN); Chengfei Pu, Shanghai (CN); Xiaogang Shen, Shanghai (CN); Haiyang Tang, Shanghai (CN); Le Tian, Rockville, MD (US)

(73) Assignees: Innovative Cellular Therapeutics Holdings, Ltd., George Town (KY); Innovative Cellular Therapeutics, Inc., Rockville, MD (US)

(21) Appl. No.: 19/427,736

(22) Filed: Dec. 19, 2025

Related U.S. Application Data
(63) Continuation of application No. 17/998,127, filed on May 7, 2021, now abandoned.
(60) Provisional application No. 63/021,983, filed on May 8, 2020.
(60) Provisional application No. 63/052,546, filed on Jul. 16, 2020.
(60) Provisional application No. 63/068,025, filed on Aug. 20, 2020.
(60) Provisional application No. 63/089,940, filed on Oct. 9, 2020.
(60) Provisional application No. 63/147,475, filed on Feb. 9, 2021.

(51) Int. Cl. A61M 1/36; C07K 16/28; C12M 1/34

(52) U.S. Cl. CPC A61M 1/362; A61M 1/3609; A61M 1/3692; C07K 16/2809; C07K 16/2818; C12M 41/36; A61M 2205/3324; A61M 2205/3368

(57) ABSTRACT

The present disclosure relates to a system for immune therapy, the system comprising a sample processing module configured to obtain whole blood from a subject; a cell incubation module configured to activate blood cells of the whole blood and/or introduce a vector into the blood cells of the whole blood; and a cell infusion module configured to infuse at least a portion of the whole blood to the subject, wherein the blood cells comprise CD3+ cells, NK cells, myeloid cells, and neutrophils.

### CROSS REFERENCE TO RELATED APPLICATIONS

This application is a continuation of co-pending U.S. patent application Ser. No. 17/998,127, filed Nov. 7, 2022, which is a U.S. National Phase Application of International Application No. PCT/US2021/031258, filed May 7, 2021, which claims the benefit of U.S. Provisional Application No. 63/021,983, filed May 8, 2020; U.S. Provisional Application 63/052,546, filed Jul. 16, 2020; U.S. Provisional Application 63/068,025, filed Aug. 20, 2020; U.S. Provisional Application No. 63/089,940, filed Oct. 9, 2020; and U.S. Provisional Application No. 63/147,475, filed Feb. 9, 2021, all of which are incorporated herein by reference in their entirety.

### SEQUENCE LISTING INFORMATION

A computer readable XML file, entitled “1071-0044USC1_ST26,” created on or about Dec. 19, 2025, with a file size of about 16,384 Bytes, contains the sequence listing for this application and is hereby incorporated by reference in its entirety.

### TECHNICAL FIELD

The present disclosure relates to compositions and methods for expanding and maintaining modified cells including genetically modified cells and uses thereof in the treatment of diseases, including cancer.

### BACKGROUND

Chimeric antigen receptor T lymphocytes (i.e., CAR T cells) identify tumor-specific markers and play a direct role in killing tumor cells. Since the first generation of CAR molecules was constructed, T Do not request examination until closer to the final due datecells expressing various CAR molecules have been widely used for treating diseases (e.g., cancers). One of the challenges of CAR T cell therapy is the development of efficient technologies and cost-effective clinical manufacturing platforms to allow safe and effective therapeutic uses.

### SUMMARY

Embodiments relate to a system for immune therapy, the system comprising: a sample processing module configured to obtain blood cells such as CD3+ cells from a blood sample from a subject; a cell incubation module configured to activate the blood cell and introduce a vector into the blood cells, and a cell infusion module configured to infuse at least a portion of the blood to the subject. In embodiments, the system is closed and/or automatic for immune therapy (e.g., gene and/or cellular therapy).

This Summary is not intended to identify key features or essential features of the claimed subject matter, nor is it intended to be used to limit the scope of the claimed subject matter.

### BRIEF DESCRIPTION OF THE DRAWINGS

The Detailed Description is described with reference to the accompanying figures. The use of the same reference numbers in different figures indicates similar or identical items.

FIG. 1 shows a schematic diagram of a process and system for immune therapy.

FIG. 2 shows growth curves of CD19 CAR T cells obtained in different manners. The cells are mixed with the vectors for 0, 2, 4, 6, 8, 12, 24, 48 hours.

FIG. 3 shows IL2 release of CD19 CAR T cells cultured with CD19 positive cells. “Stimulate” refers to the activation of T cells, for example with CD3/CD28, prior to transduction. “No Stimulate” refers to no activation.

FIG. 4 shows IL6 release of CD19 CAR T cells cultured with CD19 positive cells.

FIG. 5 shows TNF-α release of CD19 CAR T cells cultured with CD19 positive cells.

FIG. 6 shows IFNγ release of CD19 CAR T cells cultured with CD19 positive cells.

FIG. 7 shows GZMB release of CD19 CAR T cells cultured with CD19 positive cells.

FIG. 8 shows IL2 release of CD19 CAR T cells cultured with CD19 positive cells.

FIG. 9 shows IL6 release of CD19 CAR T cells cultured with CD19 positive cells.

FIG. 10 shows TNF-α release of CD19 CAR T cells cultured with CD19 positive cells.

FIG. 11 shows IFNγ release of CD19 CAR T cells cultured with CD19 positive cells.

FIG. 12 shows GZMB release of CD19 CAR T cells cultured with CD19 positive cells.

FIG. 13 shows results of functional analysis of CD19 CAR T cells cultured with CD19 positive cells.

FIG. 14 shows exemplary immune therapies.

FIG. 15 shows exemplary immune therapies.

FIG. 16 shows flow cytometry results of GFP expression by blood cells.

FIG. 17 shows flow cytometry results of GFP expression by blood cells.

FIG. 18 shows flow cytometry results of GFP expression by blood cells.

FIG. 19 shows flow cytometry results of GFP expression by blood cells.

FIG. 20 shows flow cytometry results of CAR expression in T cells.

FIG. 21 shows flow cytometry results of CAR expression in T cells.

FIG. 22 shows flow cytometry results of CAR expression in T cells.

FIG. 23 shows flow cytometry results of CAR expression in T cells.

FIG. 24 shows flow cytometry results of CAR expression in T cells.

FIG. 25 shows flow cytometry results of CAR expression in T cells.

FIG. 26 shows a schematic view of a gene/cell therapy system 200.

FIG. 27 shows a schematic view of the fluid changing module.

FIG. 28 shows a schematic view of the fluid changing device shown in FIG. 27.

FIG. 29 shows a schematic view of the infection bag for holding the blood cells transduced or transfected with the vector.

FIG. 30 shows flow diagrams of an illustrative process 2000 for implementing a cell and/or gene therapy system.

FIG. 31 shows flow diagrams of an illustrative process 2000 for implementing a cell and/or gene therapy system.

FIG. 32 shows flow cytometry assay results of whole blood transduction of vectors encoding GUCY2C CAR.

FIG. 33 shows flow cytometry assay results of whole blood transduction of vectors encoding GUCY2C CAR.

FIG. 34 shows flow cytometry assay results of whole blood transduction of vectors encoding GUCY2C CAR.

FIG. 35 shows flow cytometry assay results of whole blood transduction of vectors encoding GUCY2C CAR.

FIG. 36 shows flow cytometry assay results of whole blood transduction of vectors encoding GUCY2C CAR.

FIG. 37 shows flow cytometry assay results of whole blood transduction of vectors encoding GUCY2C CAR.

FIG. 38 shows flow cytometry assay results of whole blood transduction of vectors encoding GUCY2C CAR.

FIG. 39 shows flow cytometry assay results of whole blood transduction of vectors encoding GUCY2C CAR.

FIG. 40 shows flow cytometry assay results of whole blood transduction of vectors encoding GUCY2C CAR.

FIG. 41 shows flow cytometry assay results of whole blood transduction of vectors encoding GUCY2C CAR.

FIG. 42 shows flow cytometry assay results of whole blood transduction of vectors encoding GUCY2C CAR.

FIG. 43 shows flow cytometry assay results of whole blood transduction of vectors encoding GUCY2C CAR.

FIG. 44 shows flow cytometry assay results of whole blood transduction of vectors encoding GUCY2C CAR.

FIG. 45 shows flow cytometry assay results of whole blood transduction of vectors encoding GUCY2C CAR.

FIG. 46 shows flow cytometry assay results of whole blood transduction of vectors encoding GUCY2C CAR.

FIG. 47 shows flow cytometry assay results of whole blood transduction of vectors encoding GUCY2C CAR.

FIG. 48 shows flow cytometry assay results of whole blood transduction of vectors encoding GUCY2C CAR.

FIG. 49 shows flow cytometry assay results of whole blood transduction of vectors encoding GUCY2C CAR.

FIG. 50 shows flow cytometry assay results of whole blood transduction of vectors encoding GUCY2C CAR.

FIG. 51 shows flow cytometry assay results of whole blood transduction of vectors encoding GUCY2C CAR.

FIG. 52 shows flow cytometry assay results of whole blood transduction of vectors encoding GUCY2C CAR.

FIG. 53 shows flow cytometry assay results of whole blood transduction of vectors encoding GUCY2C CAR.

FIG. 54 shows flow cytometry assay results of whole blood transduction of vectors encoding GUCY2C CAR.

FIG. 55 shows flow cytometry assay results of whole blood transduction of vectors encoding GUCY2C CAR.

FIG. 56 shows flow cytometry assay results of whole blood transduction of vectors encoding GUCY2C CAR.

FIGS. 57A and 57B show flow cytometry results of in vivo whole blood transduction.

FIG. 58 shows a scheme for exemplary in vivo infusions.

FIG. 59 shows the proportion of GFP-infected CD3+ cells in the spleens of mice.

FIG. 60 shows results of CD19 CAR cell infection in spleens of mice.

FIG. 61 shows a scheme for the examples related to the whole blood transduction.

FIG. 62 shows the proliferation of CAR T cells in the peripheral blood of mice.

FIG. 63 shows the proliferation of tumor-bearing cells in the peripheral blood of mice.

FIG. 64 shows survival curves of mice that were infused with infected whole blood cells.

FIG. 65 shows an exemplary device for performing CAR T therapy.

### DETAILED DESCRIPTION

Unless defined otherwise, all technical and scientific terms used herein have the same meaning as commonly understood by those of ordinary skill in the art to which the disclosure belongs. Although any method and material similar or equivalent to those described herein can be used in the practice or testing of the present disclosure, preferred methods and materials are described. For the purposes of the present disclosure, the following terms are defined below.

The articles “a” and “an” are used herein to refer to one or more than one (i.e., to at least one) of the grammatical object of the article. By way of example, “an element” means one element or more than one element.

By “about” is meant a quantity, level, value, number, frequency, percentage, dimension, size, amount, weight, or length that varies by as much as 20, 15, 10, 9, 8, 7, 6, 5, 4, 3, 2 or 1% to a reference quantity, level, value, number, frequency, percentage, dimension, size, amount, weight or length.

The term “activation,” as used herein, refers to the state of a cell that has been sufficiently stimulated to induce detectable cellular proliferation. Activation can also be associated with induced cytokine production and detectable effector functions. The term “activated T cells” refers to, among other things, T cells that are undergoing cell division.

The term “antibody” is used in the broadest sense and refers to monoclonal antibodies (including full length monoclonal antibodies), polyclonal antibodies, multispecific antibodies (e.g., bispecific antibodies), and antibody fragments so long as they exhibit the desired biological activity or function. The antibodies in the present disclosure can exist in a variety of forms including, for example, polyclonal antibodies; monoclonal antibodies; Fv, Fab, Fab′, and F(ab′)2 fragments; as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “antibody fragments” refers to a portion of a full-length antibody, for example, the antigen binding or variable region of the antibody. Other examples of antibody fragments include Fab, Fab′, F(ab′)2, and Fv fragments; diabodies; linear antibodies; single-chain antibody molecules; and multispecific antibodies formed from antibody fragments.

The term “Fv” refers to the minimum antibody fragment which contains a complete antigen-recognition and -binding site. This fragment consists of a dimer of one heavy- and one light-chain variable region domain in a tight, non-covalent association. From the folding of these two domains emanates six hypervariable loops (3 loops each from the H and L chain) that contribute amino acid residues for antigen binding and confer antigen binding specificity to the antibody. However, even a single variable domain (or half of an Fv including only three complementarity determining regions (CDRs) specific for an antigen) has the ability to recognize and bind antigen, although at a lower affinity than the entire binding site (the dimer).

An “antibody heavy chain,” as used herein, refers to the larger of the two types of polypeptide chains present in all antibody molecules in their naturally occurring conformations. An “antibody light chain,” as used herein, refers to the smaller of the two types of polypeptide chains present in all antibody molecules in their naturally occurring conformations. κ and λ light chains refer to the two major antibody light chain isotypes.

The term “synthetic antibody” refers to an antibody that is generated using recombinant DNA technology, such as, for example, an antibody expressed by a bacteriophage. The term also includes an antibody that has been generated by the synthesis of a DNA molecule encoding the antibody and the expression of the DNA molecule to obtain the antibody or to obtain an amino acid encoding the antibody. The synthetic DNA is obtained using technology that is available and well known in the art.

The term “antigen” refers to a molecule that provokes an immune response, which may involve either antibody production, or the activation of specific immunologically competent cells, or both. Antigens include any macromolecule, including all proteins or peptides or molecules derived from recombinant or genomic DNA. For example, DNA including a nucleotide sequence or a partial nucleotide sequence encoding a protein or peptide that elicits an immune response, and therefore, encodes an “antigen,” as the term is used herein. An antigen need not be encoded solely by a full-length nucleotide sequence of a gene. An antigen can be generated, synthesized, or derived from a biological sample including a tissue sample, a tumor sample, a cell, or a biological fluid.

The term “anti-tumor effect,” as used herein, refers to a biological effect associated with a decrease in tumor volume, a decrease in the number of tumor cells, a decrease in the number of metastases, decrease in tumor cell proliferation, decrease in tumor cell survival, an increase in life expectancy of a subject having tumor cells, or amelioration of various physiological symptoms associated with the cancerous condition. An “anti-tumor effect” can also be manifested by the ability of the peptides, polynucleotides, cells, and antibodies in the prevention of the occurrence of tumors in the first place.

The term “auto-antigen” refers to an endogenous antigen mistakenly recognized by the immune system as being foreign. Auto-antigens include cellular proteins, phosphoproteins, cellular surface proteins, cellular lipids, nucleic acids, glycoproteins, including cell surface receptors.

The term “autologous” is used to describe a material derived from a subject that is subsequently re-introduced into the same subject.

The term “allogeneic” is used to describe a graft derived from a different subject of the same species. As an example, a donor subject may be related or unrelated to the recipient subject, but the donor subject has immune system markers which are similar to the recipient subject.

The term “xenogeneic” is used to describe a graft derived from a subject of a different species. As an example, the donor subject is from a different species than a recipient subject, and the donor subject and the recipient subject can be genetically and immunologically incompatible.

The term “cancer” is used to refer to a disease characterized by the rapid and uncontrolled growth of aberrant cells. Cancer cells can spread locally or through the bloodstream and lymphatic system to other parts of the body. Examples of various cancers include breast cancer, prostate cancer, ovarian cancer, cervical cancer, skin cancer, pancreatic cancer, colorectal cancer, renal cancer, liver cancer, brain cancer, lymphoma, leukemia, lung cancer, and the like.

Throughout this specification, unless the context requires otherwise, the words “comprise,” “includes,” and “including” will be understood to imply the inclusion of a stated step or element or group of steps or elements but not the exclusion of any other step or element or group of steps or elements.

The phrase “consisting of” is meant to include, and is limited to, whatever follows the phrase “consisting of.” Thus, the phrase “consisting of” indicates that the listed elements are required or mandatory and that no other elements may be present.

The phrase “consisting essentially of” is meant to include any element listed after the phrase and can include other elements that do not interfere with or contribute to the activity or action specified in the disclosure for the listed elements. Thus, the phrase “consisting essentially of” indicates that the listed elements are required or mandatory, and other elements may be present or optional. These other elements do not affect the activity or action of the required elements in a statistically significant manner. As an example, these other elements do not affect the ability of the required elements to kill cancer cells or to expand or maintain cells. These elements include excipients or carriers.

The terms “complementary” and “complementarity” refer to polynucleotides (i.e., a sequence of nucleotides) related to the base-pairing rules. For example, the sequence “A-G-T” is complementary to the sequence “T-C-A.” Complementarity may be “partial,” in which only some of the nucleic acids' bases are matched according to the base-pairing rules, or there may be “complete” or “total” complementarity between the nucleic acids. The degree of complementarity between nucleic acid strands has significant effects on the efficiency and strength of hybridization between nucleic acid strands.

The term “corresponds to” or “corresponding to” refers to (a) a polynucleotide having a nucleotide sequence that is substantially identical or complementary to all or a portion of a reference polynucleotide sequence or encoding an amino acid sequence identical to an amino acid sequence in a peptide or protein, or (b) a peptide or polypeptide having an amino acid sequence that is substantially identical to a sequence of amino acids in a reference peptide or protein.

The term “co-stimulatory ligand” refers to a molecule on an antigen-presenting cell (e.g., an APC, dendritic cell, B cell, and the like) that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including at least one of proliferation, activation, differentiation, and other cellular responses. A co-stimulatory ligand can include B7-1 (CD80), B7-2 (CD86), PD-L1, PD-L2, 4-1BBL, OX40L, inducible co-stimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, a ligand for CD7, an agonist or antibody that binds the Toll ligand receptor, and a ligand that specifically binds with B7-H3. A co-stimulatory ligand also includes, inter alia, an agonist or an antibody that specifically binds with a co-stimulatory molecule present on a T cell, such as CD27, CD28, 4-1BB, OX40, CD30, CD40, PD-1, ICOS, lymphocyte function-associated antigen-1 (LFA-1), CD2, CD7, LIGHT, NKG2C, B7-H3, and a ligand that specifically binds CD83.

The term “co-stimulatory molecule” refers to the cognate binding partner on a T cell that specifically binds with a co-stimulatory ligand, thereby mediating a co-stimulatory response by the T cell, such as proliferation. Co-stimulatory molecules include an MHC class I molecule, BTLA, and a Toll-like receptor.

The term “co-stimulatory signal” refers to a signal, which in combination with a primary signal, such as TCR/CD3 ligation, leads to T cell proliferation and/or upregulation or downregulation of key molecules.

The terms “disease” and “condition” may be used interchangeably or may be different in that the particular malady or condition may not have a known causative agent (so that etiology has not yet been worked out), and it is therefore not yet recognized as a disease but only as an undesirable condition or syndrome, wherein a more or less specific set of symptoms have been identified by clinicians. The term “disease” is a state of health of a subject wherein the subject cannot maintain homeostasis, and wherein if the disease is not ameliorated, then the subject's health continues to deteriorate. In contrast, a “disorder” in a subject is a state of health in which the animal is able to maintain homeostasis, but in which the animal's state of health is less favorable than it would be in the absence of the disorder. Left untreated, a disorder does not necessarily cause a further decrease in the animal's state of health.

The term “effective” refers to adequate to accomplish a desired, expected, or intended result. For example, an “effective amount” in the context of treatment may be an amount of a compound sufficient to produce a therapeutic or prophylactic benefit.

The term “encoding” refers to the inherent property of specific sequences of nucleotides in a polynucleotide, such as a gene, a cDNA, or an mRNA, to serve as a template for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence (except that a “T” is replaced by a “U”) and is usually provided in sequence listings, and the non-coding strand, used as the template for transcription of a gene or cDNA, can be referred to as encoding the protein or other product of that gene or cDNA.

The term “exogenous” refers to a molecule that does not naturally occur in a wild-type cell or organism but is typically introduced into the cell by molecular biological techniques. Examples of exogenous polynucleotides include vectors, plasmids, and/or man-made nucleic acid constructs encoding the desired protein. With regard to polynucleotides and proteins, the term “endogenous” or “native” refers to a naturally-occurring polynucleotide or amino acid sequences that may be found in a given wild-type cell or organism. Also, a particular polynucleotide sequence that is isolated from a first organism and transferred to a second organism by molecular biological techniques is typically considered an “exogenous” polynucleotide or amino acid sequence with respect to the second organism. In specific embodiments, polynucleotide sequences can be “introduced” by molecular biological techniques into a microorganism that already contains such a polynucleotide sequence, for instance, to create one or more additional copies of an otherwise naturally-occurring polynucleotide sequence, and thereby facilitate overexpression of the encoded polypeptide.

The term “expression or overexpression” refers to the transcription and/or translation of a particular nucleotide sequence into a precursor or mature protein, for example, driven by its promoter. “Overexpression” refers to the production of a gene product in transgenic organisms or cells that exceeds levels of production in normal or non-transformed organisms or cells. As defined herein, the term “expression” refers to expression or overexpression.

The term “expression vector” refers to a vector including a recombinant polynucleotide including expression control (regulatory) sequences operably linked to a nucleotide sequence to be expressed. An expression vector includes sufficient cis-acting elements for expression; other elements for expression can be supplied by the host cell or in an in vitro expression system. Expression vectors include all those known in the art, such as cosmids, plasmids (e.g., naked or contained in liposomes), and viruses (e.g., lentiviruses, retroviruses, adenoviruses, and adeno-associated viruses) that incorporate the recombinant polynucleotide.

Viruses can be used to deliver nucleic acids into a cell in vitro and in vivo (in a subject). Examples of viruses useful for the delivery of nucleic acids into cells include retrovirus, adenovirus, herpes simplex virus, vaccinia virus, and adeno-associated virus. In embodiments, various recombinant viruses may be used to deliver genetic material into target cells. For example, adenovirus can infect a variety of mammalian cell types with high efficiency. They remain epichromosal upon infection (i.e., do not integrate into the host genome), so they are only suitable for transient expression. Adenovirus can be packaged at a relatively high titer. Adeno-Associated Virus (AAV) integrates into the host cell genome at a very specific site in one human chromosome; random insertions are very rare, making it less immunogenic than other viral vectors. Like adenovirus, it can infect a variety of mammalian cell types. Overall, AAV can be produced at a moderately high titer and can infect target cells efficiently. Retrovirus (e.g., MMLV) can introduce genetic material into the genome of the host cell, making it great for long-term stable expression. Lentivirus (e.g., HIV-1, FIV, SIV) is a sub-class of retrovirus that can be used for both transient and stable gene expression. Lentivirus can infect both proliferating and non-proliferating cells and generates a low immune response in target cells. Recombinant lentivirus can be produced at a moderately high titer and is efficient in target cell transduction.

There also exist non-viral methods for delivering nucleic acids into a cell, for example, electroporation, gene gun, sonoporation, magnetofection, and the use of oligonucleotides, lipoplexes, dendrimers, and inorganic nanoparticles.

The term “homologous” refers to sequence similarity or sequence identity between two polypeptides or between two polynucleotides when a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared to ×100. For example, if 6 of 10 of the positions in two sequences are matched or homologous, then the two sequences are 60% homologous. By way of example, the DNA sequences ATTGCC and TATGGC share 50% homology. A comparison is made when two sequences are aligned to give maximum homology.

The term “immunoglobulin” or “lg” refers to a class of proteins, which function as antibodies. The five members included in this class of proteins are IgA, IgG, IgM, IgD, and IgE. IgA is the primary antibody that is present in body secretions, such as saliva, tears, breast milk, gastrointestinal secretions, and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses and is important in defense against bacteria and viruses. IgD is the immunoglobulin that has no known antibody function but may serve as an antigen receptor. IgE is the immunoglobulin that mediates immediate hypersensitivity by causing the release of mediators from mast cells and basophils upon exposure to the allergen.

The term “isolated” refers to a material that is substantially or essentially free from components that normally accompany it in its native state. The material can be a cell or a macromolecule such as a protein or nucleic acid. For example, an “isolated polynucleotide,” as used herein, refers to a polynucleotide, which has been purified from the sequences which flank it in a naturally occurring state, e.g., a DNA fragment that has been removed from the sequences that are normally adjacent to the fragment. Alternatively, an “isolated peptide” or an “isolated polypeptide” and the like, as used herein, refer to in vitro isolation and/or purification of a peptide or polypeptide molecule from its natural cellular environment and from association with other components of the cell.

The term “substantially purified” refers to a material that is substantially free from components that are normally associated with it in its native state. For example, a substantially purified cell refers to a cell that has been separated from other cell types with which it is normally associated in its naturally occurring or native state. In some instances, a population of substantially purified cells refers to a homogenous population of cells. In other instances, this term refers simply to a cell that has been separated from the cells with which they are naturally associated in their natural state. In embodiments, the cells are cultured in vitro. In embodiments, the cells are not cultured in vitro.

In the context of the present disclosure, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may, in some version, contain an intron(s).

The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. Moreover, the use of lentiviruses enables the integration of the genetic information into the host chromosome, resulting in stably transduced genetic information. HIV, SIV, and FIV are all examples of lentiviruses. Vectors derived from lentiviruses offer the means to achieve significant levels of gene transfer in vivo.

The term “modulating” refers to mediating a detectable increase or decrease in the level of a response in a subject compared with the level of a response in the subject in the absence of a treatment or compound and/or compared with the level of a response in an otherwise identical but untreated subject. The term encompasses perturbing and/or affecting a native signal or response, thereby mediating a beneficial therapeutic response in a subject, preferably a human.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence, or a ribosome binding site is operably linked to a coding sequence if it is positioned so as to facilitate translation.

The term “under transcriptional control” refers to a promoter being operably linked to and in the correct location and orientation in relation to a polynucleotide to control (regulate) the initiation of transcription by RNA polymerase and expression of the polynucleotide.

The term “overexpressed” tumor antigen or “overexpression” of the tumor antigen is intended to indicate an abnormal level of expression of the tumor antigen in a cell from a disease area such as a solid tumor within a specific tissue or organ of the patient relative to the level of expression in a normal cell from that tissue or organ. Patients having a solid tumor or a hematological malignancy characterized by overexpression of the tumor antigen can be determined by standard assays known in the art.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme), astrocytoma, CNS lymphoma, germinoma, medulloblastoma, Schwannoma craniopharyngioma, ependymoma, pinealoma, hemangioblastoma, acoustic neuroma, oligodendroglioma, meningioma, neuroblastoma, retinoblastoma, and brain metastases).

A solid tumor antigen is an antigen expressed on a solid tumor. In embodiments, solid tumor antigens are also expressed at low levels on healthy tissue. Examples of solid tumor antigens and their related disease tumors are provided in Table 1.

TABLE 1

| Solid Tumor antigen | Disease tumor |
|---|---|
| PRLR | Breast cancer |
| CLCA1 | colorectal cancer |
| MUC12 | colorectal cancer |
| GUCY2C | colorectal cancer |
| GPR35 | colorectal cancer |
| CR1L | Gastric cancer |
| MUC 17 | Gastric cancer |
| TMPRSS11B | esophageal cancer |
| MUC21 | esophageal cancer |
| TMPRSS11E | esophageal cancer |
| CD207 | bladder cancer |
| SLC30A8 | pancreatic cancer |
| CFC1 | pancreatic cancer |
| SLC12A3 | Cervical cancer |
| SSTR1 | Cervical tumor |
| GPR27 | Ovary tumor |
| FZD10 | Ovary tumor |
| TSHR | Thyroid Tumor |
| SIGLEC15 | Urothelial cancer |
| SLC6A3 | Renal cancer |
| KISS1R | Renal cancer |
| QRFPR | Renal cancer: |
| GPR119 | Pancreatic cancer |
| CLDN6 | Endometrial cancer/Urothelial |
| | cancer |
| UPK2 | Urothelial cancer (including |
| | bladder cancer) |
| ADAM12 | Breast cancer, pancreatic cancer, |
| | and the like |
| SLC45A3 | Prostate cancer |
| ACPP | Prostate cancer |
| MUC21 | Esophageal cancer |
| MUC16 | Ovarian cancer |
| MS4A12 | Colorectal cancer |
| ALPP | Endometrial cancer |
| CEA | Colorectal carcinoma |
| EphA2 | Glioma |
| FAP | Mesothelioma |
| GPC3 | Lung squamous cell carcinoma |
| IL13-Rα2 | Glioma |
| Mesothelin | Metastatic cancer |
| PSMA | Prostate cancer |
| ROR1 | Breast lung carcinoma |
| VEGFR-II | Metastatic cancer |
| GD2 | Neuroblastoma |
| FR-α | Ovarian carcinoma |
| ErbB2 | Carcinoma |
| EpCAM | Carcinoma |
| EGFRvIII | Glioma-Glioblastoma |
| EGFR | Glioma-NSCL cancer |
| tMUC1 | Cholangiocarcinoma, Pancreatic |
| | cancer, Breast |
| PSCA | pancreas, stomach, or prostate |
| | cancer |
| FCER2, GPR18, FCRLA, | breast cancer |
| CXCR5, FCRL3, FCRL2, | |
| HTR3A, and CLEC17A | |
| TRPMI, SLC45A2, and | lymphoma |
| SLC24A5 | |
| DPEP3 | melanoma |
| KCNK16 | ovarian, testis |
| LIM2 or KCNV2 | pancreatic |
| SLC26A4 | thyroid cancer |
| CD171 | Neuroblastoma |
| Glypican-3 | Sarcoma |
| IL-13 | Glioma |
| CD79a or CD79b | Lymphoma |
| MAGE A4 | Lung and other cancer types |

The term “parenteral administration” of a composition includes, e.g., subcutaneous (s.c.), intravenous (i.v.), intramuscular (i.m.), intrasternal injection, or infusion techniques.

The terms “patient,” “subject,” and “individual,” and the like are used interchangeably herein and refer to any human or animal, amenable to the methods described herein. In certain non-limiting embodiments, the patient, subject, or individual is a human or animal. In embodiments, the term “subject” is intended to include living organisms in which an immune response can be elicited (e.g., mammals). Examples of subjects include humans, and animals, such as dogs, cats, mice, rats, and transgenic species thereof.

A subject in need of treatment or in need thereof includes a subject having a disease, condition, or disorder that needs to be treated. A subject in need thereof also includes a subject that needs treatment for the prevention of a disease, condition, or disorder, for example, cancer.

The term “polynucleotide” or “nucleic acid” refers to mRNA, RNA, CRNA, rRNA, cDNA, or DNA. The term typically refers to a polymeric form of nucleotides of at least 10 bases in length, either ribonucleotides or deoxynucleotides, or a modified form of either type of nucleotide. The term includes all forms of nucleic acids, including single and double-stranded forms of nucleic acids.

The terms “polynucleotide variant” and “variant,” and the like refer to polynucleotides displaying substantial sequence identity with a reference polynucleotide sequence or polynucleotides that hybridize with a reference sequence under stringent conditions that are defined hereinafter. These terms also encompass polynucleotides that are distinguished from a reference polynucleotide by the addition, deletion, or substitution of at least one nucleotide. Accordingly, the terms “polynucleotide variant” and “variant” include polynucleotides in which one or more nucleotides have been added or deleted, or replaced with different nucleotides. In this regard, it is well understood in the art that certain alterations inclusive of mutations, additions, deletions, and substitutions can be made to a reference polynucleotide whereby the altered polynucleotide retains the biological function or activity of the reference polynucleotide or has increased activity in relation to the reference polynucleotide (i.e., optimized). Polynucleotide variants include, for example, polynucleotides having at least 50% (and at least 51% to at least 99% and all integer percentages in between, e.g., 90%, 95%, or 98%) sequence identity with a reference polynucleotide sequence described herein. The terms “polynucleotide variant” and “variant” also include naturally-occurring allelic variants and orthologs.

The terms “polypeptide,” “polypeptide fragment,” “peptide,” and “protein” are used interchangeably herein to refer to a polymer of amino acid residues and to variants and synthetic analogues of the same. Thus, these terms apply to amino acid polymers in which one or more amino acid residues are synthetic non-naturally occurring amino acids, such as a chemical analogue of a corresponding naturally occurring amino acid, as well as to naturally occurring amino acid polymers. In certain aspects, polypeptides may include enzymatic polypeptides, or “enzymes,” which typically catalyze (i.e., increase the rate of) various chemical reactions.

The term “polypeptide variant” refers to polypeptides that are distinguished from a reference polypeptide sequence by the addition, deletion, or substitution of at least one amino acid residue. In certain embodiments, a polypeptide variant is distinguished from a reference polypeptide by one or more substitutions, which may be conservative or non-conservative. In certain embodiments, the polypeptide variant comprises conservative substitutions, and, in this regard, it is well understood in the art that some amino acids may be changed to others with broadly similar properties without changing the nature of the activity of the polypeptide. Polypeptide variants also encompass polypeptides in which one or more amino acids have been added or deleted, or replaced with different amino acid residues.

The term “promoter” refers to a DNA sequence recognized by the synthetic machinery of the cell or introduced synthetic machinery required to initiate the specific transcription of a polynucleotide sequence. The term “expression control (regulatory) sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

The term “bind,” “binds,” or “interacts with” refers to a molecule recognizing and adhering to a second molecule in a sample or organism but does not substantially recognize or adhere to other structurally unrelated molecules in the sample. The term “specifically binds,” as used herein with respect to an antibody, refers to an antibody that recognizes a specific antigen but does not substantially recognize or bind other molecules in a sample. For example, an antibody that specifically binds an antigen from one species may also bind that antigen from one or more species. But, such cross-species reactivity does not itself alter the classification of an antibody as specific. In another example, an antibody that specifically binds an antigen may also bind different allelic forms of the antigen. However, such cross-reactivity does not itself alter the classification of an antibody as specific. In some instances, the terms “specific binding” or “specifically binding” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds a specific protein structure rather than to any protein. If an antibody is specific for epitope “A,” the presence of a molecule containing epitope A (or free, unlabeled A) in a reaction containing labeled “A” and the antibody will reduce the amount of labeled A bound to the antibody.

By “statistically significant,” it is meant that the result was unlikely to have occurred by chance. Statistical significance can be determined by any method known in the art. Commonly used measures of significance include the p-value, which is the frequency or probability with which the observed event would occur if the null hypothesis were true. If the obtained p-value is smaller than the significance level, then the null hypothesis is rejected. In simple cases, the significance level is defined at a p-value of 0.05 or less. A “decreased” or “reduced” or “lesser” amount is typically a “statistically significant” or a physiologically significant amount and may include a decrease that is about 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 1.5, 1.6, 1.7. 1.8, etc.) an amount or level described herein.

The term “stimulation” refers to a primary response induced by binding of a stimulatory molecule (e.g., a TCR/CD3 complex) with its cognate ligand, thereby mediating a signal transduction event, such as signal transduction via the TCR/CD3 complex. Stimulation can mediate altered expression of certain molecules, such as downregulation of TGF-β and/or reorganization of cytoskeletal structures.

The term “stimulatory molecule” refers to a molecule on a T cell that specifically binds a cognate stimulatory ligand present on an antigen-presenting cell. For example, a functional signaling domain derived from a stimulatory molecule is the zeta chain associated with the T cell receptor (TCR) complex. The stimulatory molecule includes a domain responsible for signal transduction.

The term “stimulatory ligand” refers to a ligand that when present on an antigen-presenting cell (e.g., an APC, a dendritic cell, a B-cell, and the like.) can specifically bind with a cognate binding partner (referred to herein as a “stimulatory molecule”) on a cell, for example, a T cell, thereby mediating a primary response by the T cell, including activation, initiation of an immune response, proliferation, and similar processes. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

The term “therapeutic” refers to the treatment and/or prophylaxis. A therapeutic effect is obtained by suppression, remission, or eradication of a disease state or alleviating the symptoms of a disease state.

The term “therapeutically effective amount” refers to the amount of the subject compound that will elicit the biological or medical response of a tissue, system, or subject that is being sought by the researcher, veterinarian, medical doctor, or another clinician. The term “therapeutically effective amount” includes that amount of a compound that, when administered, is sufficient to prevent the development of, or alleviate to some extent, one or more of the signs or symptoms of the disorder or disease being treated. The therapeutically effective amount will vary depending on the compound, the disease, and its severity, and the age, weight, etc., of the subject to be treated.

The term “treat a disease” refers to the reduction of the frequency or severity of at least one sign or symptom of a disease or disorder experienced by a subject.

The term “transfected” or “transformed” or “transduced” refers to a process by which an exogenous nucleic acid is transferred or introduced into the host cell. A “transfected” or “transformed” or “transduced” cell is one that has been transfected, transformed, or transduced with an exogenous nucleic acid. The cell includes the primary subject cell and its progeny.

The term “vector” refers to a polynucleotide that comprises an isolated nucleic acid and which can be used to deliver the isolated nucleic acid to the interior of a cell. Numerous vectors are known in the art including linear polynucleotides, polynucleotides associated with ionic or amphiphilic compounds, plasmids, and viruses. Thus, the term “vector” includes an autonomously replicating plasmid or a virus. The term also includes non-plasmid and non-viral compounds which facilitate the transfer of nucleic acid into cells, such as, for example, polylysine compounds, liposomes, and the like. Examples of viral vectors include adenoviral vectors, adeno-associated virus vectors, retroviral vectors, and others. For example, lentiviruses are complex retroviruses, which, in addition to the common retroviral genes gag, pol, and env, contain other genes with regulatory or structural function. Lentiviral vectors are well known in the art. Some examples of lentivirus include the Human Immunodeficiency Viruses: HIV-1, HIV-2, and the Simian Immunodeficiency Virus: SIV. Lentiviral vectors have been generated by multiply attenuating the HIV virulence genes, for example, the genes env, vif, vpr, vpu, and nef are deleted, making the vector biologically safe.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, and 6. This applies regardless of the breadth of the range.

A “chimeric antigen receptor” (CAR) molecule is a recombinant polypeptide including at least an extracellular domain, a transmembrane domain, and a cytoplasmic domain or intracellular domain. In embodiments, the domains of the CAR are on the same polypeptide chain, for example, a chimeric fusion protein. In embodiments, the domains are on different polypeptide chains, for example, the domains are not contiguous.

The extracellular domain of a CAR molecule includes an antigen binding domain. The antigen binding domain is for expanding and/or maintaining the modified cells, such as a CAR T cell, or for killing a tumor cell, such as a solid tumor. In embodiments, the antigen binding domain for expanding and/or maintaining modified cells binds an antigen, for example, a cell surface molecule or marker, on the surface of a WBC.

In embodiments, the WBC is at least one of GMP (granulocyte macrophage precursor), MDP (monocyte-macrophage/dendritic cell precursors), cMoP (common monocyte precursor), basophil, eosinophil, neutrophil, SatM (Segerate-nucleus-containing atypical monocyte), macrophage, monocyte, CDP (common dendritic cell precursor), cDC (conventional DC), pDC (plasmacytoid DC), CLP (common lymphocyte precursor), B cell, ILC (Innate Lymphocyte), NK cell, megakaryocyte, myeloblast, pro-myelocyte, myelocyte, meta-myelocyte, band cells, lymphoblast, prolymphocyte, monoblast, megakaryoblast, promegakaryocyte, megakaryocyte, platelets, or MSDC (Myeloid-derived suppressor cell). In embodiments, the WBC is a granulocyte, monocyte, and or lymphocyte. In embodiments, the WBC is a lymphocyte, for example, a B cell. In embodiments, the WBC is a B cell. In embodiments, the cell surface molecule of a B cell includes CD19, CD22, CD20, BCMA, CD5, CD7, CD2, CD16, CD56, CD30, CD14, CD68, CD11b, CD18, CD169, CD1c, CD33, CD38, CD138, or CD13. In embodiments, the cell surface molecule of the B cell is CD19, CD20, CD22, or BCMA. In embodiments, the cell surface molecule of the B cell is CD19.

The cells described herein, including modified cells such as CAR cells including CAR T cells, and modified T cells, can be derived from stem cells. Stem cells may be adult stem cells, embryonic stem cells, more particularly non-human stem cells, cord blood stem cells, progenitor cells, bone marrow stem cells, induced pluripotent stem cells, totipotent stem cells, or hematopoietic stem cells. A modified cell may also be a dendritic cell, an NK-cell, a B-cell, or a T cell selected from the group consisting of inflammatory T-lymphocytes, cytotoxic T-lymphocytes, regulatory T lymphocytes, or helper T-lymphocytes. In embodiments, Modified cells may be derived from the group consisting of CD4+ T lymphocytes and CD8+ T lymphocytes. Prior to the expansion and genetic modification of the cells of the invention, a source of cells may be obtained from a subject through a variety of non-limiting methods. T cells may be obtained from a number of non-limiting sources, including peripheral blood mononuclear cells, bone marrow, lymph node tissue, cord blood, thymus tissue, tissue from a site of infection, ascites, pleural effusion, spleen tissue, and tumors. In certain embodiments of the present invention, any number of T cell lines available and known to those skilled in the art may be used. In embodiments, modified cells may be derived from a healthy donor, from a patient diagnosed with cancer, or from a patient diagnosed with an infection. In embodiments, a modified cell is part of a mixed population of cells that present different phenotypic characteristics.

A population of cells refers to a group of two or more cells. The cells of the population could be the same, such that the population is a homogenous population of cells. The cells of the population could be different, such that the population is a mixed population or a heterogeneous population of cells. For example, a mixed population of cells could include modified cells comprising a first CAR and cells comprising a second CAR, wherein the first CAR and the second CAR bind different antigens.

The term “stem cell” refers to any of certain types of cell which have the capacity for self-renewal and the ability to differentiate into other kind(s) of a cell. For example, a stem cell gives rise either to two daughter stem cells (as occurs in vitro with embryonic stem cells in culture) or to one stem cell and a cell that undergoes differentiation (as occurs, e.g., in hematopoietic stem cells, which give rise to blood cells). Different categories of stem cells may be distinguished on the basis of their origin and/or on the extent of their capacity for differentiation into other types of cells. For example, stem cells may include embryonic stem (ES) cells (i.e., pluripotent stem cells), somatic stem cells, induced pluripotent stem cells, and any other types of stem cells.

The pluripotent embryonic stem cells are found in the inner cell mass of a blastocyst and have an innate capacity for differentiation. For example, pluripotent embryonic stem cells have the potential to form any type of cell in the body. When grown in vitro for long periods of time, ES cells maintain pluripotency as progeny cells retain the potential for multilineage differentiation.

Somatic stem cells can include fetal stem cells (from the fetus) and adult stem cells (found in various tissues, such as bone marrow). These cells have been regarded as having a capacity for differentiation that is lower than that of the pluripotent ES cells—with the capacity of fetal stem cells being greater than that of adult stem cells. Somatic stem cells differentiate into only a limited number of types of cells and have been described as multipotent. The “tissue-specific”stem cells normally give rise to only one type of cell. For example, embryonic stem cells may be differentiated into blood stem cells (e.g., Hematopoietic stem cells (HSCs)), which may be further differentiated into various blood cells (e.g., red blood cells, platelets, white blood cells, etc.).

Induced pluripotent stem cells (i.e., iPS cells or iPSCs) may include a type of pluripotent stem cell artificially derived from a non-pluripotent cell (e.g., an adult somatic cell) by inducing an expression of specific genes. Induced pluripotent stem cells are similar to natural pluripotent stem cells, such as embryonic stem (ES) cells, in many aspects, such as the expression of certain stem cell genes and proteins, chromatin methylation patterns, doubling time, embryoid body formation, teratoma formation, viable chimera formation, and potency and differentiability. Induced pluripotent cells can be obtained from adult stomach, liver, skin, and blood cells.

In embodiments, the antigen binding domain for killing a tumor binds an antigen on the surface of a tumor, for example, a tumor antigen or tumor marker. Tumor antigens are proteins that are produced by tumor cells that elicit an immune response, particularly T cell-mediated immune responses. Tumor antigens are well known in the art and include, for example, tumor associated MUC1 (tMUC1), a glioma-associated antigen, carcinoembryonic antigen (CEA), β-human chorionic gonadotropin, alpha fetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, surviving, telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor, CD19, and mesothelin. For example, when the tumor antigen is CD19, the CAR thereof can be referred to as CD19 CAR or 19CAR, which is a CAR molecule that includes an antigen binding domain that binds CD19.

In embodiments, the extracellular antigen binding domain of a CAR includes at least one scFv or at least a single domain antibody. As an example, there can be two scFvs on a CAR. The scFv includes a light chain variable (VL) region and a heavy chain variable (VH) region of a target antigen-specific monoclonal antibody joined by a flexible linker. Single chain variable region fragments can be made by linking light and/or heavy chain variable regions by using a short linking peptide (Bird et al., Science 242:423-426, 1988). An example of a linking peptide is the GS linker having the amino acid sequence (GGGGS)3 (SEQ ID NO: 8), which bridges approximately 3.5 nm between the carboxy terminus of one variable region and the amino terminus of the other variable region. Linkers of other sequences have been designed and used (Bird et al., 1988, supra). In general, linkers can be short, flexible polypeptides and preferably comprised of about 20 or fewer amino acid residues. The single chain variants can be produced either recombinantly or synthetically. For the synthetic production of scFv, an automated synthesizer can be used. For recombinant production of scFv, a suitable plasmid containing a polynucleotide that encodes the scFv can be introduced into a suitable host cell, either eukaryotic, such as yeast, plant, insect, or mammalian cells, or prokaryotic, such as E. coli. Polynucleotides encoding the scFv of interest can be made by routine manipulations such as ligation of polynucleotides. The resultant scFv can be isolated using standard protein purification techniques known in the art.

The cytoplasmic domain of the CAR molecules described herein includes one or more co-stimulatory domains and one or more signaling domains. The co-stimulatory and signaling domains function to transmit the signal and activate molecules, such as T cells, in response to antigen binding. The one or more co-stimulatory domains are derived from stimulatory molecules and/or co-stimulatory molecules, and the signaling domain is derived from a primary signaling domain, such as the CD3 zeta domain. In embodiments, the signaling domain further includes one or more functional signaling domains derived from a co-stimulatory molecule. In embodiments, the co-stimulatory molecules are cell surface molecules (other than antigens receptors or their ligands) that are required for activating a cellular response to an antigen.

In embodiments, the co-stimulatory domain includes the intracellular domain of CD27, CD28, 4-1BB, OX40, CD30, CD40, PD-1, ICOS, lymphocyte function-associated antigen-1 (LFA-1), CD2, CD7, LIGHT, NKG2C, B7-H3, a ligand that specifically binds with CD83, or any combination thereof. In embodiments, the signaling domain includes a CD3 zeta domain derived from a T cell receptor.

The CAR molecules described herein also include a transmembrane domain. The incorporation of a transmembrane domain in the CAR molecules stabilizes the molecule. In embodiments, the transmembrane domain of the CAR molecules is the transmembrane domain of a CD28 or 4-1BB molecule.

A spacer domain can be incorporated between the extracellular domain and the transmembrane domain of the CAR. As used herein, the term “spacer domain” generally means any oligo- or polypeptide that functions to link the transmembrane domain to the extracellular domain and/or the cytoplasmic domain on the polypeptide chain. A spacer domain can include up to 300 amino acids, preferably 10 to 100 amino acids, and most preferably 25 to 50 amino acids.

In embodiments, the intracellular domain comprises a co-stimulatory signaling region that comprises an intracellular domain of a co-stimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, and any combination thereof.

In embodiments, the antigen is Epidermal growth factor receptor (EGFR), Variant III of the epidermal growth factor receptor (EGFRvIII), Human epidermal growth factor receptor 2 (HER2), Mesothelin (MSLN), Prostate-specific membrane antigen (PSMA), Carcinoembryonic antigen (CEA), Disialoganglioside 2 (GD2), Interleukin-13Ra2 (IL13Rα2), Glypican-3 (GPC3), Carbonic anhydrase IX (CAIX), L1 cell adhesion molecule (L1-CAM), Cancer antigen 125 (CA125), Cluster of differentiation 133 (CD133), Fibroblast activation protein (FAP), Cancer/testis antigen 1B (CTAG1B), Mucin 1 (MUC1), Folate receptor-α (FR-α), CD19, FZD10, TSHR, PRLR, Muc 17, GUCY2C, CD207, CD3, CD5, B-Cell Maturation Antigen (BCMA), or CD4.

In embodiments, the vector comprises a nucleic acid sequence encoding a binding molecule (e.g., an antigen binding molecule). In embodiments, the binding molecule is a CAR or TCR. In embodiments, the vector is a lentivirus. In embodiments, the lentivirus can be packaged with a particle (e.g., a Nano particle) such as to be released for a predetermined time and directly infused to the subject such that the lentivirus can be transferred to T cells of the subject.

In embodiments, the antigen binding molecule is a CAR or a T Cell Receptor (TCR). The term “Chimeric Antigen Receptor” or alternatively a “CAR” refers to a recombinant polypeptide construct comprising at least an extracellular antigen binding domain, a transmembrane domain, and an intracellular signaling domain (e.g., cytoplasmic domain). In embodiments, the domains in the CAR polypeptide construct are in the same polypeptide chain (e.g., comprising a chimeric fusion protein) or not contiguous with each other (e.g., in different polypeptide chains).

In embodiments, the intracellular signaling domain can include a functional signaling domain derived from a stimulatory molecule and/or a co-stimulatory molecule as described above. In certain embodiments, the intracellular signaling domain includes a functional signaling domain derived from a primary signaling domain (e.g., a primary signaling domain of CD3-zeta). In other embodiments, the intracellular signaling domain further includes one or more functional signaling domains derived from at least one co-stimulatory molecule. The co-stimulatory signaling region refers to a portion of the CAR, including the intracellular domain of a co-stimulatory molecule. Co-stimulatory molecules are cell surface molecules other than antigens receptors or their ligands that are required for an efficient response of lymphocytes to antigen.

Between the extracellular domain and the transmembrane domain of the CAR, there can be incorporated a spacer domain. As used herein, the term “spacer domain” generally means any oligo- or polypeptide that functions to link the transmembrane domain to either the extracellular domain or the cytoplasmic domain in the polypeptide chain. A spacer domain can include up to 300 amino acids, preferably 10 to 100 amino acids, and most preferably 25 to 50 amino acids.

The extracellular domain of a CAR can include an antigen binding domain (e.g., an scFv, a single domain antibody, or TCR (e.g., a TCR alpha binding domain or TCR beta binding domain)) that targets a specific tumor marker (e.g., a tumor antigen). Tumor antigens are proteins that are produced by tumor cells that elicit an immune response, particularly T cell-mediated immune responses. Tumor antigens are well known in the art and include, for example, a glioma-associated antigen, carcinoembryonic antigen (CEA), β-human chorionic gonadotropin, alpha fetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor, and mesothelin. For example, the tumor antigen is CD19, and the CAR thereof may be referred to as CD19 CAR.

In embodiments, the extracellular ligand-binding domain comprises an scFv comprising the light chain variable (VL) region and the heavy chain variable (VH) region of a target antigen-specific monoclonal antibody joined by a flexible linker. Single chain variable region fragments are made by linking light and/or heavy chain variable regions by using a short linking peptide (Bird et al., Science 242:423-426, 1988). An example of a linking peptide is the GS linker having the amino acid sequence (GGGGS)3 (SEQ ID NO: 8), which bridges approximately 3.5 nm between the carboxy terminus of one variable region and the amino terminus of the other variable region. Linkers of other sequences have been designed and used (Bird et al., 1988, supra). In general, linkers can be short, flexible polypeptides and preferably comprised of about 20 or fewer amino acid residues. Linkers can, in turn, be modified for additional functions, such as attachment of drugs or attachment to solid supports. The single chain variants can be produced either recombinantly or synthetically. For the synthetic production of scFv, an automated synthesizer can be used. For recombinant production of scFv, a suitable plasmid containing a polynucleotide that encodes the scFv can be introduced into a suitable host cell, either eukaryotic, such as yeast, plant, insect, or mammalian cells, or prokaryotic, such as E. coli. Polynucleotides encoding the scFv of interest can be made by routine manipulations such as ligation of polynucleotides. The resultant scFv can be isolated using standard protein purification techniques known in the art.

In embodiments, the tumor antigen includes HER2, CD19, CD20, CD22, Kappa or light chain, CD30, CD33, CD123, CD38, ROR1, ErbB3/4, EGFR, EGFRvIII, EphA2, FAP, carcinoembryonic antigen, EGP2, EGP40, mesothelin, TAG72, PSMA, NKG2D ligands, B7-H6, IL-13 receptor α 2, IL-11 receptor α, MUC1, MUC16, CA9, GD2, GD3, HMW-MAA, CD171, Lewis Y, G250/CAIX, HLA-AI MAGE A1, HLA-A2 NY-ESO-1, PSC1, folate receptor-α, CD44v7/8, 8H9, NCAM, VEGF receptors, 5T4, Fetal AchR, NKG2D ligands, CD44v6, TEM1, TEM8, or viral-associated antigens expressed by the tumor.

More information about CARs and their uses can be found in PCT Patent Publications WO2020106843 and WO2019140100 and in PCT Patent Application NO: PCT/US20/13099, which are incorporated herein by reference in their entirety.

“NFAT promoter” refers to one or more NFAT responsive elements linked to a minimal promoter of any gene expressed by T cells. In embodiments, the minimal promoter of a gene expressed by T cells is a minimal human IL2 promoter. The NFAT responsive elements can comprise, e.g., NFAT1, NFAT2, NFAT3, and/or NFAT4 responsive elements. The NFAT promoter (or a functional portion or functional variant thereof) can comprise any number of binding motifs, e.g., at least two, at least three, at least four, at least five, or at least six, at least seven, at least eight, at least nine, at least ten, at least eleven, or up to twelve binding motifs. In embodiments, the NFAT promoter comprises six NFAT binding motifs.

The NFAT promoter (or a functional portion or functional variant thereof) is operatively associated with the nucleotide sequence encoding a target protein (or a functional portion or functional variant thereof). “Operatively associated with” means that the nucleotide sequence encoding target protein (or a functional portion or functional variant thereof) is transcribed into target protein mRNA when the NFAT protein binds to the NFAT promoter sequence (or a functional portion or functional variant thereof). Without being bound to a particular theory, it is believed that NFAT is regulated by a calcium signaling pathway. In particular, it is believed that TCR stimulation (by, e.g., an antigen) and/or stimulation of the calcium signaling pathway of the cell (by, e.g., PMA/lonomycin) increases intracellular calcium concentration and activates calcium channels. It is believed that the NFAT protein is then dephosphorylated by calmodulin and translocates to the nucleus, where it binds with the NFAT promoter sequence (or a functional portion or functional variant thereof) and activates downstream gene expression. By providing an NFAT promoter (or a functional portion or functional variant thereof) that is operatively associated with the nucleotide sequence encoding target protein (or a functional portion or functional variant thereof), the nucleic acids of the invention advantageously make it possible to express target protein (or a functional portion or functional variant thereof) only when the host cell including the nucleic acid is stimulated by, e.g., PMA/lonomycin and/or an antigen. More information can be found at U.S. Pat. No. 8,556,882, which is incorporated by reference in its entirety.

Embodiments relate to a system for immune therapy, the system comprising: a sample processing module configured to obtain CD3+ cells from a blood sample from a subject; a cell incubation module configured to activate CD3+ cell and introduce a vector into the CD3+ cells, and a cell infusion module configured to infuse at least a portion of the CD3+ to the subject.

The sample processing module can further comprise a unit configured to obtain human cells from the peripheral blood of a subject. For example, the unit is an apheresis device in which the blood of a person is passed through an apparatus that separates out one particular constituent and returns the remainder to the circulation.

In embodiments, the sample separation module can purify CD3+ cells by negative selection using, e.g., the RosetteSep T cell enrichment Cocktail or positive selection using anti-CD3 coupled to magnetic particles. Following isolation, CD3+ cells were cultured in X-VIVO 15 (Cambrex, Walkersville, MD) supplemented with 5% normal human AB serum (Valley Biomedical, Winchester, VA), 2 mM L-glutamine (Cambrex), 20 mM HEPES (Cambrex), and IL2 (100 units/mL; R&D Systems). In some embodiments, the blood sample can be diluted using Dulbecco phosphate buffered saline (DPBS), and apheresis density gradient centrifugation can be performed by the sample separation module to obtain PBMCs containing lymphocytes. MACS buffer can be used to rinse the PBMCs, and Pan T Cell-Ab cocktail can be mixed with PBMCs and incubated for 5 min. Pan T cell beads cocktail can be added and incubated at 2-8 degrees for 10 min. In embodiments, the sample separation module can include or be coupled to LS columns used to collect CD3+ cells. In embodiments, the output port can be configured to infuse the remaining components of the blood sample or non-desired cells (e.g., CD3-cells) back to the subject and to flow the desired cells (e.g., CD3+ cells) to the cell incubation module.

As used herein, the term “flow” or “flowing” with respect to the cells, components of the blood, and/or fluids refers to moving or transporting them from one location to another.

The cell incubation module can be configured for stimulation/activation and culturing of the CD3+ cells. For example, the CD3+ cells can be stimulated with magnetic beads precoated with agonist antibodies against CD3 and CD28 at a ratio of three beads per cell, and then resuspended at a concentration of 106 CD+3 cells/mL for expansion for a predetermined time (e.g., 2-20 hours). CD3+ cells can be then transduced with vectors including nucleic acids encoding CD19-BBζ CAR (CD19 4-1BB CD3 zeta CAR) hours after the cell stimulation and expansion for a predetermined time (e.g., 1-5 hours) or harvested at specific time points for analysis. CD3+ cells can be maintained in culture at a concentration of 0.1 to 1×106 cells/mL by adjusting the concentration based on counting by flow cytometry using antibodies to human CD4 and CD8. After completion of cell culture, the magnetic beads can be removed and tested for release criteria specified for T cell phenotype, cell viability (≥70%), concentration (≥80% CD3+CD45+), and transduction efficiency (≥2%). In embodiments, CD3+ cells can be co-cultured with beads conjugated with CD19 antigen comprising a His tag (e.g., 6-histidines attached to the C terminal of the CD19 antigen), after removal of beads conjugated with anti-CD3 and anti-CD28 and transduction with lentiviral virus containing a nucleic acid encoding an anti-CD19 CAR (i.e., CD19 CAR).

The cell infusion module can comprise an input port, an output port, and a rotating container. The input port can be configured to obtain the CD3+ cells from the cell incubation module; the rotating container can be configured to remove beads (e.g., magnetic beads precoated with agonist antibodies against CD3 and CD28 or CD19 beads) and replace cultural media of the CD+3 cells with media suitable for infusion; and the output port can be configured to provide the CD+3 cell for the infusion to the subject.

In embodiments, the sample processing module is coupled or connected to the cell incubation module that is coupled or connected to the cell infusion module such that the CD3+ cells flow through the system starting from the subject and ending back to the subject.

In embodiments, the sample processing module comprises: an input port configured to receive the blood sample from the subject; a sample separation module configured to: separate the CD3+ cells from remaining components of the blood sample, and collect the CD3+ cells; and an output port configured to flow the CD3+ cells to the cell incubation module, and flow the remaining components of the blood sample to the subject.

In embodiments, the sample separation module is configured to contact the blood sample with a composition comprising a CD3 aggregation reagent comprising CD3 recognizing moiety coupled to a magnetic particle; apply gravity sedimentation for sedimentation of CD3+ cells, and a magnetic field gradient to the blood sample for immobilizing the magnetic particle simultaneously; generate a pellet and a supernatant phase; and recover the desired cells from the pellet. In embodiments, the sample separation module is configured to contact the blood sample with a composition comprising antigen recognizing moieties specifically binding undesired cellular components; apply gravity sedimentation for sedimentation of undesired cells, and a magnetic field gradient to the blood sample for immobilizing the magnetic particle simultaneously; generate a pellet and a supernatant phase; and recover the desired cells from the supernatant phase.

In embodiments, the system comprises a material made of plastic. The plastic can be selected from the group consisting of polystyrol, polystyrene, polyvinylchloride, polycarbonate, glass, polyacrylate, polyacrylamide, polymethylmethacrylate (PMMA), polyethylene terephthalate (PET), polytetrafluoroethylene (PTFE), thermoplastic polyurethane (TPU), silicone, polyethylene (PE), collagen, chitin, alginate, hyaluronic acid derivatives, polylactide (PLA), polyglycolide (PGA) and their copolymers, polystyrol, polystyrene, polycarbonate, polyacrylate, ceramics, glass materials such as hydroxyapatite (HA) and calcium phosphate, and compositions comprising one or more of the above-mentioned materials.

In embodiments, the system further comprises one or more sensors configured to monitor the progress of separation of the blood sample from the subject, in particular by detecting the formation of layers of the blood sample, the change of pH value of the blood sample, and/or the change of temperature of the blood sample.

In embodiments, the cell incubation module comprises a rotating container configured to culture cells and/or grow cells. The rotating container is disposable and/or has been sterilized.

Embodiments relate to a method of immune therapy by implementing the system, the method comprising: receiving peripheral blood of a subject; collecting CD3+ cells from the peripheral blood; returning the remaining cells of the peripheral blood to the subject; obtaining vectors comprising a nucleic acid sequence encoding a CAR and one or more agents that activate T cells; mixing the vectors with the obtained CD3+ cells and incubating the mixture for a predetermined time to introduce the nucleic acid sequence into the CD3+ cells; and replacing culture media of the transfected CD3+ cells with a solution suitable for infusion into the subject, and providing at least a portion of the transfected CD3+ cells to the subject.

In embodiments, the method is completed in less than 1, 2, 3, 4, or 5 days.

Embodiments relate to an apparatus for sample processing, the apparatus comprising: an input port configured to receive blood sample from a subject; a sample separation module configured to isolate desired cells from the blood sample, an output port configured to: flow the isolated desired cells for further processing, and return the rest of the cells and/or remaining components of the blood sample to the subject.

In embodiments, the sample processing module can further comprise a unit configured to obtain human cells from the peripheral blood of the subject. In embodiments, the unit is an apheresis device in which the blood of a person is passed through an apparatus that separates out one particular constituent and returns the remainder to the circulation of the subject.

Embodiments relate to a method for sample processing by implementing the apparatus, the method comprising: receiving a blood sample from a subject; isolating desired cells from the blood sample; flowing the desired cells for further processing; returning the rest of the cells and/or remaining components of the blood sample to the subject; introducing a nucleic acid encoding an antigen binding molecule into the desired cell to obtain a transfected cell; and administering an effective amount of a composition comprising the transfected cells. In embodiments, the desired cells are CD3+ cells.

Embodiments relate to a method of delivering a therapeutic protein to the peripheral blood system of a subject, the method comprising: administering to the peripheral blood system of the subject, a viral vector (e.g., retrovirus, adenovirus, adeno associated virus, and/or Lentivirus vectors) comprising a nucleic acid sequence encoding the therapeutic protein, such that cells, organ, or tissue of the peripheral blood system expresses the therapeutic protein in the subject. The therapeutic protein can be an antigen binding molecule.

Embodiments relate to a method of treating cancer in a subject, the method comprising: administering to the peripheral blood system of the subject a viral vector comprising a nucleic acid sequence encoding a CAR molecule such that cells, organ or tissue of the peripheral blood system express the CAR molecule in the subject; and monitoring T cell response resulting from the expression of CAR molecule in the subject. Embodiments relate to a method of in vivo expression of antigen binding molecule in lymphocytes of a subject, the method comprising: administering to the peripheral blood system of the subject a viral vector comprising a nucleic acid sequence encoding an antigen binding molecule such that cells, organ or tissue of the peripheral blood system express the antigen binding molecule of the subject.

Adeno associated virus (AAV) is a small nonpathogenic virus of the parvoviridae family. AAV is distinct from other members of this family by its dependence upon a helper virus for replication. In the absence of a helper virus, AAV can integrate in a locus-specific manner into the q arm of chromosome 19. Approximately 5 kb genome of AAV consists of one segment of single-stranded DNA of either plus or minus polarity. The ends of the genome are short inverted terminal repeats which can fold into hairpin structures and serve as the origin of viral DNA replication. Physically, the parvovirus virion is non-enveloped, and its icosahedral capsid is approximately 20-30 nm in diameter.

In general, an adeno-associated virus (AAV) includes three related proteins referred to as VPL protein and two shorter proteins, called VP2 and VP3, that are essentially amino-terminal truncations of VPL. Depending on the capsid and other factors known to those of ordinary skill, the three capsid proteins VPL, VP2 and VP3 are typically present in the capsid at a ratio approximating 1:1:10, respectively, although this ratio, particularly the amount of VP3, can vary significantly and should not to be considered limiting in any respect. The ends of the AAV genome have short inverted terminal repeats (ITR), which have the potential to fold into T-shaped hairpin structures that serve as the origin of viral DNA replication. Within the ITR region, two elements have been described, which are central to the function of the ITR, a GAGC repeat motif and the terminal resolution site (TRS). The repeated motif has been shown to bind Rep (replication protein) when the ITR is in either a linear or hairpin conformation. This binding serves to position Rep68/78 for cleavage at the TRS, which occurs in a site- and strand-specific manner. In addition to their role in replication, the GAGC repeat motif and the TRS appear to be central to viral integration. Contained within the chromosome 19 integration locus is a Rep binding site with an adjacent TRS. The GAGC repeat motif and the TRS have been shown to be functional for locus-specific integration. AAV are useful as gene therapy vectors as they can penetrate cells and introduce nucleic acid/genetic material so that the nucleic acid/genetic material can be stably maintained in cells.

In addition, these adeno-associated viruses (AAVs) can introduce nucleic acid/genetic material into specific sites, for example, a specific site on chromosome 19. Because AAV is not associated with pathogenic disease in humans, AAV vectors are able to deliver heterologous polynucleotide sequences encoding therapeutic proteins and agents to human patients without causing substantial AAV pathogenesis or disease. Accordingly, recombinant AAV (rAAV) vectors, including serotypes and variants, provide a means for the delivery of nucleic acids encoding proteins into cells ex vivo, in vitro, and in vivo, such that the cells express the encoded proteins. For example, an rAAV vector can include a heterologous nucleic acid encoding a desired protein or peptide. Vector delivery or administration to a subject (e.g., mammal) provides the encoded protein to the subject. More Information on gene therapy and its clinical uses can be found at U.S. Pat. Nos. 9,433,688, 9,644,216, and Ser. No. 10/113,183 as well as Milone, M. C., O'Doherty, U. Clinical use of lentiviral vectors. Leukemia 32, 1529-1541 (2018), Rodrigues, G. A., Shalaev, E., Karami, T. K. et al. Pharmaceutical Development of AAV-Based Gene Therapy Products for the Eye. Pharm Res 36, 29 (2019), and Marquez Loza, L. I.; Yuen, E. C.; McCray, P. B., Jr. Lentiviral Vectors for the Treatment and Prevention of Cystic Fibrosis Lung Disease. Genes 2019, 10, 218, which are incorporated by reference in their entirety.

Modified AAV vectors, such as recombinant nucleic acids and polypeptides, mean that the composition (e.g., AAV or sequences) has been manipulated (i.e., engineered) in a manner that it generally does not occur in nature. A particular example of a recombinant AAV vector would be one in which a nucleic acid that is not normally present in the wild-type viral (e.g., AAV) genome (“heterologous”) is inserted within the viral genome. A “recombinant” AAV vector is distinguished from an AAV genome since all, or a part of the viral genome has been replaced with a non-native sequence with respect to the AAV genomic nucleic acid, such as a heterologous nucleic acid sequence. Although the term “recombinant” is not always used herein in reference to AAV vectors, as well as its sequences such as nucleic acids and polypeptides, recombinant forms of AAV, and sequences including nucleic acids and polypeptides, are expressly included in spite of any such omission. Typically for AAV, one or both inverted terminal repeat (ITR) sequences of the AAV genome are retained in the AAV vector. Incorporation of a non-native sequence therefore defines the AAV vector as a “recombinant” AAV (rAAV) vector.

A recombinant AAV vector can be packaged as a “particle” for subsequent transduction of a cell, for example, ex vivo, in vitro, or in vivo transduction. Where a recombinant AAV vector sequence is encapsidated or packaged into an AAV particle, the particle can also be referred to herein as an “rAAV.” Such particles include proteins that encapsidate or package the vector genomes, and in the case of AAV, capsid proteins. An “AAV viral particle” or “AAV particle” refers to a viral particle composed of at least one AAV capsid protein (typically all of the capsid proteins of an AAV) and an encapsidated nucleic acid, referred to as a vector genome. If the particle comprises heterologous nucleic acid, it is typically referred to as “rAAV.”

An AAV vector “genome” refers to the portion of the recombinant plasmid sequence that is ultimately packaged or encapsidated to form an AAV particle. In cases where recombinant plasmids are used to construct or manufacture recombinant AAV vectors, the AAV vector genome does not include the portion of the “plasmid” that does not correspond to the vector genome sequence of the recombinant plasmid. This non-vector genome portion of the recombinant plasmid is referred to as the “plasmid backbone,” which is important for cloning and amplification of the plasmid, a process that is needed for propagation and recombinant AAV production but is not itself packaged or encapsidated into rAAV particles.

In embodiments, rAAV vectors include capsids derived from AAV1, AAV2, AAV3, AAV4, AAV5, AAV6, AAV7, AAV8, AAV9, AAV10, AAV11, RhIO, Rh74, or AAV-2i8, as well as variants {e.g., capsid variants, such as amino acid insertions, additions and substitutions) thereof. AAV vector serotypes and variants include capsid variants (e.g., LK03, 4-1, etc). rAAV serotypes and rAAV variants (e.g., capsid variants such as LK03, 4-1) may or may not be distinct from other AAV serotypes (e.g., distinct from VP1, VP2, and/or VP3 sequences). The term “serotype” is a distinction used to refer to an AAV having a capsid that is serologically distinct from other AAV serotypes. Serologic distinctiveness is determined based on the lack of cross-reactivity between antibodies to one AAV as compared to another AAV. Such cross-reactivity differences are usually due to differences in capsid protein sequences/antigenic determinants (e.g., due to VP1, VP2, and/or VP3 sequence differences of AAV serotypes). Despite the possibility that AAV variants, including capsid variants, may not be serologically distinct from a reference AAV or other AAV serotype, they differ by at least one nucleotide or amino acid residue compared to the reference or other AAV serotype.

Recombinant AAV vector (e.g., rAAV), as well as methods and uses thereof, include any viral strain or serotype. As a non-limiting example, a recombinant AAV vector genome can be based upon any AAV genome, such as AAV-1, -2, -3, -4, -5, -6, -7, -8, -9, -10, -11, -rh74, -rhIO or AAV-2i8. Such vectors can be based on the same strain or serotype (or subgroup or variant) or be different from each other. As a non-limiting example, a recombinant AAV vector genome based upon one serotype genome can be identical to one or more of the capsid proteins that package the vector. In addition, a recombinant AAV vector genome can be based upon an AAV (e.g., AAV2) serotype genome distinct from one or more of the capsid proteins that package the vector, in which case at least one of the three capsid proteins can be an AAV1, AAV2, AAV3, AAV4, AAV5, AAV6, AAV7, AAV8, AAV9, AAV10, AAV11, RhIO, Rh74 or AAV-218 or variant (e.g., capsid variants such as LK03, 4-1, etc.). AAV vectors, therefore, include gene/protein sequences identical to gene/protein sequences characteristic for a particular serotype. As used herein, an “AAV vector related to AAV1” refers to one or more AAV proteins (e.g., VP1, VP2, and/or VP3 sequences) that have substantial sequence identity to one or more polynucleotides or polypeptide sequences that comprise AAVI. Analogously, an “AAV vector related to AAV8” refers to one or more AAV proteins (e.g., VP1, VP2, and/or VP3 sequences) that have substantial sequence identity to one or more polynucleotides or polypeptide sequences that comprise AAV8. An “AAV vector related to AAV-Rh74” refers to one or more AAV proteins (e.g., VP1, VP2, and/or VP3 sequences) that have substantial sequence identity to one or more polynucleotides or polypeptide sequences that comprise AAV-Rh74 (see, e.g., VP1, VP2, VP3). Such AAV vectors, which can be related to another serotype, for example, AAVI, AAV2, AAV3, AAV4, AAV5, AAV6, AAV7, AAV8, AAV9, AAV10, AAVI 1, RhIO, Rh74, or AAV-2i8, can therefore have one or more distinct sequences from AAVI, AAV2, AAV3, AAV4, AAV5, AAV6, AAV7, AAV8, AAV9, AAV10, AAV11, RhIO, Rh74, and AAV-218, but can exhibit substantial sequence identity to one or more genes and/or proteins, and/or have one or more functional characteristics of AAVI, AAV2, AAV3, AAV4, AAV5, AAV6, AAV7, AAV8, AAV9, AAV10, AAV11, RhIO, Rh74 or AAV-218 (e.g., such as cell/tissue tropism). Exemplary non-limiting AAV-Rh74 and related AAV variants include capsid variant 4-1 in Example 6.

An “AAV ITR” or “AAV ITRs” refers to the art-recognized regions found at each end of the AAV genome which function together in cis as origins of DNA replication and as packaging signals for the virus. AAV ITRs, together with the AAV rep coding region, provide for the efficient excision and rescue from and integration of a nucleotide sequence interposed between two flanking ITRs into a mammalian cell genome.

The nucleotide sequences of AAV ITRs are known. An “AAV ITR” need not have the wild-type nucleotide sequence depicted, but may be altered, e.g., by the insertion, deletion, or substitution of nucleotides. Additionally, the AAV ITR can be derived from any of several AAV serotypes. Furthermore, 5′ and 3′ ITRs which flank a heterologous nucleic acid sequence in an AAV vector need not necessarily be identical or derived from the same AAV serotype or isolate, so long as they function as intended, i.e., to allow for excision and rescue of the sequence of interest from a host cell genome or vector, and to allow integration of the heterologous sequence into the recipient cell genome.

AAV “empty capsids” as used herein do not contain a vector genome (hence, the term “empty”), in contrast to “genome containing capsids,” which contain an AAV vector genome. Empty capsids are virus-like particles in that they react with one or more antibodies that react with the intact (genome containing AAV vector) virus. AAV empty capsids are believed to bind to or react with antibodies against the AAV vectors, thereby functioning as a decoy to reduce inactivation of the AAV vector. Such a decoy acts to absorb antibodies directed against the AAV vector, thereby increasing or improving AAV vector transgene transduction of cells (introduction of the transgene), and in turn, increased cellular expression of the transcript and/or encoded protein. Empty capsids can be generated and purified to the desired quality, and their quantities can be determined. For example, empty capsid titer can be measured by spectrophotometry by optical density at 280 nm wavelength (based on Sommer et al., Mol. Ther. 2003 January; 7 (I): 122-8). Empty-AAV or empty capsids are sometimes naturally found in AAV vector preparations. Such natural mixtures can be used as described herein or if desired, be manipulated to increase or decrease the amount of empty capsid and/or vector. For example, the amount of empty capsid can optionally be adjusted to an amount that would be expected to reduce the inhibitory effect of antibodies that react with an AAV vector that is intended to be used for vector-mediated gene transduction in the subject. The use of empty capsids is described in US Publication 2014/0336245. In various embodiments, AAV empty capsids are formulated with rAAV vectors and/or administered to a subject. In particular aspects, AAV empty capsids are formulated with less than or an equal amount of vector (e.g., about 1.0 to 100-fold AAV vectors to AAV empty capsids, or about a 1:1 ratio of AAV vectors to AAV empty capsids). In other particular aspects, AAV vectors are formulated with an excess of AAV empty capsids (e.g., greater than 1-fold AAV empty capsids to AAV vectors, e.g., 1.0 to 100-fold AAV empty capsids to AAV vectors).

In embodiments, the pair of ITRs are derived from or comprises a sequence of any of AAV1, AAV2, AAV3, AAV4, AAV5, AAV6, AAV7, AAV8, AAV9, AAV10, AAV11, AAV 12, LK01, LK02, LK03, AAV 4-1, AAV-218 ITRs, or a mixture thereof.

In embodiments, the AAV capsid protein is derived from or comprise a sequence any of: AAV1, AAV2, AAV3, AAV4, AAV5, AAV6, AAV7, AAV8, AAV9, AAV10, AAV11, AAV 12, LK01, LK02, LK03, AAV 4-1, AAV-218 ITRs, or a mixture thereof.

A “unit dose” or a “unit dosage form” as used herein refers to physically discrete units suited as unitary dosages for the subject to be treated; each unit containing a predetermined quantity optionally in association with a pharmaceutical carrier (excipient, diluent, vehicle or filling agent) which, when administered in one or more doses, is calculated to produce the desired effect (e.g., prophylactic or therapeutic effect). Unit dosage forms can be within, for example, ampules and vials, which can include a liquid composition or a composition in a freeze-dried or lyophilized state; a sterile liquid carrier, for example, can be added prior to administration or delivery in vivo. Individual unit dosage forms can be included in multi-dose kits or containers. rAAV vectors, rAAV particles, and pharmaceutical compositions thereof can be packaged in single or multiple unit dosage forms for ease of administration and uniformity of dosage.

In embodiments, the nucleic acid encoding the therapeutic protein described herein has reduced cytosine-guanine dinucleotide (CpG) as compared to a non-CpG reduced nucleic acid encoding the therapeutic protein. In embodiments, the nucleic acid encoding the therapeutic protein described herein has reduced cytosine-guanine dinucleotide (CpG) compared to a wild-type nucleic acid encoding the therapeutic protein.

In embodiments, the rAAV vector further comprises one or more of an intron, an expression control element, a filler polynucleotide sequence and/or poly A signal, or a combination thereof.

In embodiments, the rAAV particles are administered at a dose in a range from about 1×108-1×1010, 1×1010-1×1011, 1×1011-1×1012, 1×1012-1×1013, or 1×1013-1×1014 vector genomes per kilogram (vg/kg) of the subject. In embodiments, the rAAV particles are administered at the dose of less than 1×10 vector genomes per kilogram (vg/kg) of the subject.

In embodiments, the rAAV particles are administered at a dose of about 5×1011 vector genomes per kilogram (vg/kg) of the subject. In embodiments, the rAAV particles administered are at least 1×1010 vector genomes (vg) per kilogram (vg/kg) of the weight of the subject, or between about 1×1010 to 1×1011 vg/kg of the weight of the subject, or between about 1×1011 to 1×1012 vg/kg (e.g., about 1×1011 to 2×1011 vg/kg or about 2×1011 to 3×1011 vg/kg or about 3×1011 to 4×1011 vg/kg or about 4×1011 to 5×1011 vg/kg or about 5×1011 to 6×1011 vg/kg or about 6×1011 to 7×1011 vg/kg or about 7×1011 to 8×1011 vg/kg or about 8×1011 to 9×1011 vg/kg or about 9×1011 to 1×1012 vg/kg) of the weight of the subject, or between about 1×1010 to 1×1012 vg/kg of the weight of the subject, to achieve a desired therapeutic effect.

In embodiments, the subject does not develop a substantial immune response against the rAAV particle.

In embodiments, the subject does not develop a substantial humoral immune response against the rAAV particle.

In embodiments, the subject does not develop a substantial immune response against the rAAV particle for at least 1, 2, 3, 4, 5, 6, 7, 8, 9, 10, 11, 12, 13, or 14 continuous days, weeks or months.

In embodiments, the subject does not develop a detectable immune response against the rAAV particle.

In embodiments, the subject does not produce an immune response against the therapeutic protein and/or the rAAV particle sufficient to block the therapeutic effect in the subject for at least 1, 2, 3, 4, 5, 6, 7, 8, 9, 10, 11, 12, 13 or 14 continuous days, weeks or months.

More information on AAV vectors, production of vectors, pharmaceutical compositions including AAV vectors, and methods thereof can be found in U.S. Pat. No. 9,433,688, which is incorporated by reference in its entirety.

In embodiments, the viral vector is an rAAV particle comprising an AAV capsid protein and a vector comprising the nucleic acid encoding antigen binding molecule inserted between a pair of AAV inverted terminal repeats (ITRs) in a manner effective to infect the cells, organ, or tissue of the peripheral system in the subject such that the cells, organ or tissue express antigen binding molecule.

In embodiments, administering the viral vector to a subject comprises infusion or injection of the viral vector into the systemic circulation of the subject. In embodiments, the administering comprises intravenous or intra-arterial infusion or injection into the systemic circulation of the subject.

In embodiments, the AAV is used to deliver an antigen binding molecule to a subject. The antigen binding molecule is a CAR that comprises an extracellular domain, a transmembrane domain, and an intracellular domain, and the extracellular domain binds an antigen.

In embodiments, the intracellular domain comprises a co-stimulatory signaling region that comprises an intracellular domain of a co-stimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, and any combination thereof.

In embodiments, the antigen is Epidermal growth factor receptor (EGFR), Variant III of the epidermal growth factor receptor (EGFRvIII), Human epidermal growth factor receptor 2 (HER2), Mesothelin (MSLN), Prostate-specific membrane antigen (PSMA), Carcinoembryonic antigen (CEA), Disialoganglioside 2 (GD2), Interleukin-13Ra2 (IL13Rα2), Glypican-3 (GPC3), Carbonic anhydrase IX (CAIX), L1 cell adhesion molecule (L1-CAM), Cancer antigen 125 (CA125), Cluster of differentiation 133 (CD133), Fibroblast activation protein (FAP), Cancer/testis antigen 1B (CTAG1B), Mucin 1 (MUC1), Folate receptor-α (FR-α), CD19, FZD10, TSHR, PRLR, Muc 17, GUCY2C, CD207, CD3, CD5, B-Cell Maturation Antigen (BCMA), or CD4.

In embodiments, the antigen binding molecule is a T Cell Receptor (TCR). In embodiments, the TCR is modified TCR. In embodiments, the TCR is derived from spontaneously occurring tumor-specific T cells in patients. In embodiments, the TCR binds to a tumor antigen. In embodiments, the tumor antigen comprises CEA, gp100, MART-1, p53, MAGE-A3, or NY-ESO-1. In embodiments, the TCR comprises TCRγ and TCRδ chains or TCRα and TCRβ chains. In embodiments, a T cell clone that expresses a TCR with a high affinity for the target antigen can be isolated. In embodiments, tumor-infiltrating lymphocytes (TILs) or peripheral blood mononuclear cells (PBMCs) can be cultured in the presence of antigen-presenting cells (APCs) pulsed with a peptide representing an epitope known to elicit a dominant T cell response when presented in the context of a defined HLA allele. High-affinity clones can be then selected based on MHC-peptide tetramer staining and/or the ability to recognize and lyse target cells pulsed with low titrated concentrations of cognate peptide antigen. After the clone has been selected, the TCRα and TCRβ chains or TCRγ and TCRδ Chains are identified and isolated by molecular cloning. For example, for TCRα and TCRβ chains, the TCRα and TCRβ gene sequences are then used to generate an expression construct that ideally promotes stable, high-level expression of both TCR chains in human T cells. The transduction vehicle (e.g., a gammaretrovirus or lentivirus) can be then generated and tested for functionality (antigen specificity and functional avidity) and used to produce a clinical lot of the vector. An aliquot of the final product is then used to transduce the target T cell population (generally purified from patient PBMCs), which is expanded before infusion into the patient.

In embodiments, the binding element of the CAR can include any antigen binding moiety that, when bound to its cognate antigen, affects a tumor cell such that the tumor cell fails to grow or is promoted to die or diminish.

The nucleic acid sequences encoding for the desired molecules, such as CAR molecules, can be obtained using recombinant methods known in the art, such as, for example, by screening libraries from cells expressing the gene, by deriving the gene from a vector known to include the same, or by isolating directly from cells and tissues containing the same, using standard techniques. Alternatively, the gene of interest can be produced synthetically rather than cloned.

The expression of natural or synthetic nucleic acids encoding CARs is typically achieved by operably linking a nucleic acid encoding the CAR polypeptide or portions thereof to one or more promoters and incorporating the construct into an expression vector. The vectors can be suitable for the replication and integration of eukaryotes. Typical cloning vectors contain transcription and translation terminators, initiation sequences, and promoters useful for regulation of the expression of the desired nucleic acid sequence.

Additional information related to the expression of synthetic nucleic acids encoding CARs and gene transfer into mammalian cells is provided in U.S. Pat. No. 8,906,682, incorporated by reference in its entirety.

The embodiments of the present disclosure further relate to vectors in which a DNA encoding a desired molecule of the present disclosure can be inserted. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

Pharmaceutical compositions of the present disclosure can be administered in a manner appropriate to the disease to be treated (or prevented). The quantity and frequency of administration will be determined by such factors as the condition of the patient, and the type and severity of the patient's disease, although appropriate dosages can be determined by clinical trials. As an example, pharmaceutical compositions disclosed herein include nucleic acids encoding CAR or vectors described herein and a pharmaceutically acceptable carrier.

The term “pharmaceutically acceptable” means approved by a regulatory agency of the U.S. Federal or a state government or the EMA (European Medicines Agency) or listed in the U.S. Pharmacopeia (United States Pharmacopeia-33/National Formulary-28 Reissue, published by the United States Pharmacopeial Convention, Inc., Rockville Md., publication date: April 2010) or other generally recognized pharmacopeia for use in animals, and more particularly in humans.

The term “carrier” refers to a diluent, adjuvant {e.g., Freund's adjuvant (complete and incomplete)), excipient, or vehicle with which the therapeutic is administered. Pharmaceutical carriers can be sterile liquids, such as water and oils, including those of petroleum, animal, vegetable, or synthetic origins, such as peanut oil, soybean oil, mineral oil, sesame oil, and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. For the use of (further) excipients and their use see also “Handbook of Pharmaceutical Excipients”, fifth edition, R. C. Rowe, P. J. Seskey and S. C. Owen, Pharmaceutical Press, London, Chicago.

The administration of the pharmaceutical compositions described herein can be carried out in any convenient manner, including by aerosol inhalation, injection, ingestion, transfusion, implantation, or transplantation. The compositions described herein can be administered to a patient subcutaneously, intradermally, intratumorally, intranodally, intramedullary, intramuscularly, by intravenous (i. v.) injection, or intraperitoneally. In embodiments, the T cell compositions of the present disclosure are administered to a patient by intradermal or subcutaneous injection. In another embodiment, the T cell compositions of the present disclosure are preferably administered by i.v. injection. The compositions of T cells can be injected directly into a tumor, lymph node, or site of infection. In certain embodiments of the present disclosure, cells activated and expanded using the methods described herein, or other methods known in the art where T cells are expanded to therapeutic levels, are administered to a patient in conjunction with (e.g., before, simultaneously, or following) any number of relevant treatment modalities, including but not limited to treatment with agents such as antiviral therapy, cidofovir, and interleukin-2, Cytarabine (also known as ARA-C) or natalizumab treatment for MS patients or efalizumab treatment for psoriasis patients or other treatments for PML patients. In further embodiments, the T cells of the present disclosure can be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludarabine, cyclosporin, FK506, rapamycin, mycophenolic acid, steroids, FR901228, cytokines, and irradiation. These drugs inhibit either the calcium-dependent phosphatase calcineurin (cyclosporine and FK506) or inhibit the p70S6 kinase that is important for growth factor-induced signaling (rapamycin). (Liu et al., Cell 66:807-815, 1991; Henderson et al., Immun 73:316-321, 1991; Bierer et al., Curr. Opin. Immun 5:763-773, 1993; Isoniemi (supra)). In embodiments, the cell compositions of the present disclosure are administered to a patient in conjunction with (e.g., before, simultaneously, or following) bone marrow transplantation, T cell ablative therapy using either chemotherapy agents such as fludarabine, external-beam radiation therapy (XRT), cyclophosphamide, or antibodies such as OKT3 or CAMPATH. In other embodiments, the cell compositions of the present disclosure are administered following B-cell ablative therapy, such as agents that react with CD20, e.g., Rituxan. For example, in embodiments, subjects can undergo standard treatment with high dose chemotherapy followed by peripheral blood stem cell transplantation. In certain embodiments, following the transplant, subjects receive an infusion of the expanded immune cells of the present disclosure. In other embodiments, expanded cells are administered before or following surgery.

The dosage of the above treatments to be administered to a patient will vary with the precise nature of the condition being treated and the recipient of the treatment. The scaling of dosages for human administration can be performed according to art-accepted practices by a physician, depending on various factors.

When “an immunologically effective amount,” “an anti-tumor effective amount,” “a tumor-inhibiting effective amount,” or “therapeutic amount” is indicated, the precise amount of the compositions of the present disclosure to be administered can be determined by a physician with consideration of individual differences in age, weight, tumor size, the extent of infection or metastasis, and condition of the patient (subject). It can be stated that a pharmaceutical composition comprising the T cells described herein can be administered at a dosage of 104 to 109 cells/kg body weight, preferably 105 to 106 cells/kg body weight, including all integer values within those ranges. T cell compositions can also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly. In certain embodiments, it can be desired to administer activated T cells to a subject and then subsequently redraw the blood (or have apheresis performed), collect the activated and expanded T cells, and reinfuse the patient with these activated and expanded T cells. This process can be carried out multiple times every few weeks. In certain embodiments, T cells can be activated from blood draws of from 10 cc to 400 cc. In certain embodiments, T cells are activated from blood draws of 20 cc, 30 cc, 40 cc, 50 cc, 60 cc, 70 cc, 80 cc, 90 cc, or 100 cc. Not to be bound by theory, using this multiple blood draw/multiple reinfusion protocols can select out certain populations of T cells.

Additional information on the methods of cancer treatment using engineered or modified T cells is provided in U.S. Pat. No. 8,906,682, incorporated by reference in its entirety.

Embodiments relate to an in vitro method for preparing modified cells. The method can include obtaining a sample of cells from the subject. For example, the sample can include T cells or T cell progenitors. The method can further include transfecting or transducing the cells with a DNA encoding at least a CAR, culturing the population of CAR cells ex vivo in a medium that selectively enhances proliferation of CAR-expressing T cells.

In embodiments, the sample is a cryopreserved sample. In embodiments, the sample of cells is from umbilical cord blood or a peripheral blood sample from the subject. In embodiments, the sample of cells is obtained by apheresis or venipuncture. In embodiments, the sample of cells is a subpopulation of T cells.

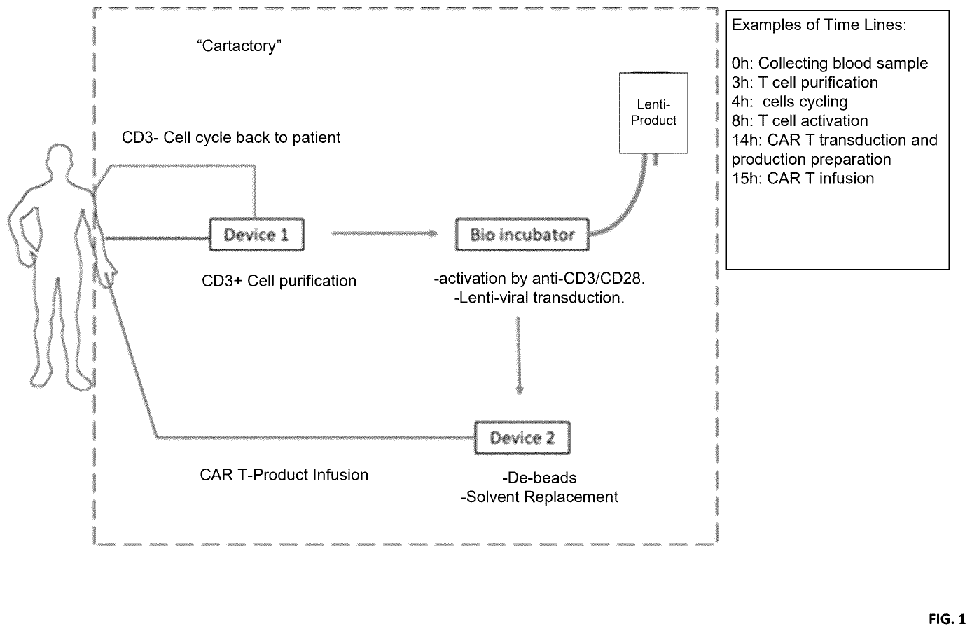

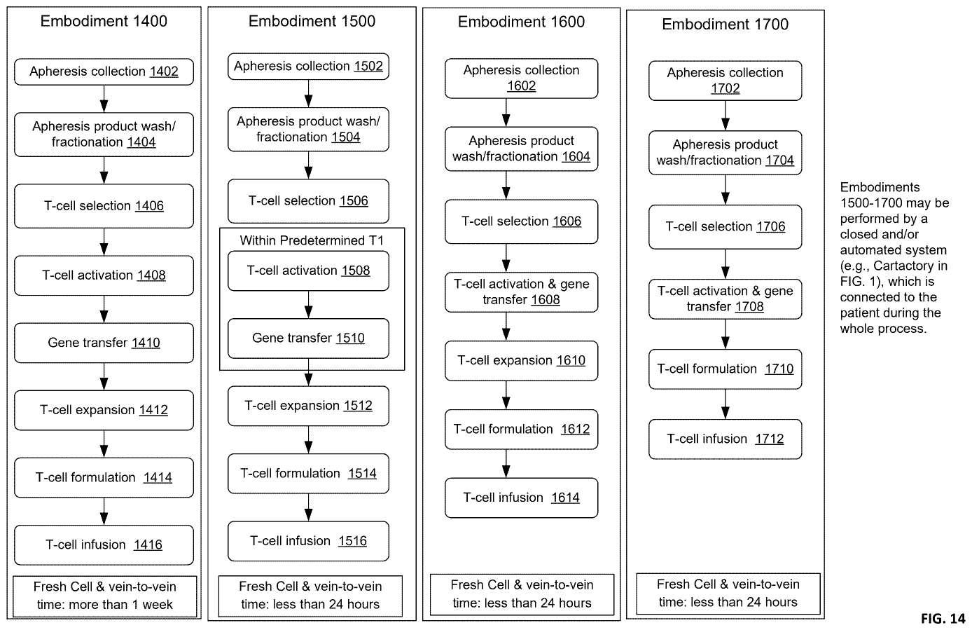

FIG. 14 shows examples of immune therapy, which include several embodiments having multiple operations. The order in which the operations are described is not intended to be construed as a limitation, and any number of the described blocks can be combined in any order or removed and/or in parallel to implement the process. Other processes described throughout this disclosure, in addition to Embodiments 1400, 1500, 1600, and 1700, shall be interpreted accordingly. The Embodiments are described with reference to the system shown in FIG. 1. However, the Embodiments can be implemented using other schemes, in other environments, and/or with immune therapy systems. Embodiment 1400 shows a conventional process for CAR T therapy. More information can be found at Molecular Therapy, Oncolytics (2016) 3, 16015; Wang X, Rivière I. Clinical manufacturing of CAR T cells: foundation of a promising therapy. Mol Ther Oncolytics. 2016 Jun. 15; 3:16015, which are incorporated herein by reference in their entirety. If fresh cells are used, vein-to-vein time is generally more than a week. Embodiments 1500, 1600, and 1700 show enhanced immune therapy based on CAR T technology. For example, in Embodiment 1500, the predetermined T1 can be 0.1-10 hours, which means that T cell activation and gene transfer can be performed within the predetermined time T1. Alternatively, T cell activation and gene transfer can be performed at the same time, as shown in Embodiments 1600 and 1700. For example, the isolated T cells can be mixed with lentivirus vectors encoding CARs and therapeutic agents and activation agents (e.g., CD3/CD28 beads). In embodiments, T cell expansion in vitro can be reduced since the therapeutic agents (e.g., IL-6, IL-12, and IFNγ) can enhance cell expansion in the subject's body. In embodiments, several steps in Embodiment 1700 can be modified or removed. For example, PBMC can be mixed with lentivirus vectors and activation agents directly. Thus, either modified PMBC or isolated modified T cells can be infused back to the subject. Table 2 shows examples of vectors. The system herein can be applied to other immune therapies (e.g., TIL/TCR). Embodiments 1500-1700 can be performed by a closed and/or automated system (e.g., Cartactory (a CAR T Factory) in FIG. 1), which is connected to the patient during the whole process. In embodiments, if fresh cells are used, vein-to-vein times for Embodiments 1500-1700 can be completed in less than 1 hr, 12 hrs, 24 hrs, 48 hrs, or 72 hrs (including numbers in between). In embodiments, the closed system is attached to the subject such that the subject's material (e.g., infused blood) will be limited to the closed system, and no operation related to the material infused back to the subject will be performed out of the system.

The present disclosure describes a method of providing modified cells. The method can comprise obtaining whole blood cells from a subject or a healthy donor, mixing a nucleic acid encoding a CAR or a TCR with the whole blood cells to obtain the modified cells. In embodiments, the modified cells comprise neutrophils. It has been reported that neutrophils promote the production of IL-12 by macrophages, induce UTCαβ into type I activation state, release IFNγ, and exert anti-tumor immune function. In embodiments, the modified cells comprise myeloid cells. In embodiments, the modified cells further comprise a polynucleotide encoding IL-12. Several markers can be used to remain myeloid cells in the blood sample, and examples of the markers include CD33, CD156a, and CD11b. In embodiments, B cells can be removed from the whole blood to reduce the risk of generation of B cell line cancer. For example, CD19 or CD20 antibodies can be incorporated into the system 200.

The present disclosure describes a method of delivering a therapeutic protein to the peripheral blood system of a subject, the method comprising: administering to the peripheral blood system of the subject a viral vector comprising a nucleic acid encoding the therapeutic protein such that cells, organ or tissue of the peripheral blood system express the antigen binding molecule of the subject. In embodiments, the therapeutic protein is an antigen binding molecule. In embodiments, the antigen binding molecule is a CAR.

The present disclosure describes a method of treating cancer in a subject, the method comprising: administering to the peripheral blood system of the subject a viral vector comprising a nucleic acid sequence encoding an antigen binding molecule such that cells, organ, or tissue of the peripheral blood system express the antigen binding molecule of the subject; and monitoring T cell response derived from the expression of antigen binding molecule in the subject.

The present disclosure describes a method of in vivo expression of antigen binding molecule in lymphocytes of a subject, the method comprising: administering into the peripheral blood system of the subject a viral vector comprising a nucleic acid sequence encoding a CAR, such that cells, organ or tissue of the peripheral blood system express the CAR of the subject.

In embodiments, the nucleic acid sequence encodes a humanized CD19 CAR described in PCT Publication No: WO2018126369, which is incorporated by its entirety.

In embodiments, the nucleic acid sequence encodes a CAR targeting an antigen of a non-essential tissue described in PCT Publication No: WO2018064921, which is incorporated herein by reference in its entirety.

In embodiments, the nucleic acid sequence encodes a CAR and a therapeutic agent described in PCT Publication No: WO2020106843, which is incorporated herein by reference in its entirety.

In embodiments, the nucleic acid sequence encodes one or more components of CoupledCAR® described in PCT Publication No: WO2020146743, which is incorporated herein by reference in its entirety.

In embodiments, the nucleic acid sequence encodes a modified immune checkpoint molecule (e.g., PD-1) as described in U.S. Pat. No. 9,572,837, which is incorporated herein by reference in its entirety. An immune checkpoint molecule refers to a molecule that is associated with the T cells and regulates T cell response. In embodiments, the immune checkpoint molecule is selected from the group consisting of PD-1, cytotoxic T lymphocyte antigen-4 (CTLA-4), B- and T-lymphocyte attenuator (BTLA), T cell immunoglobulin mucin-3 (TIM-3), lymphocyte-activation protein 3 (LAG-3), T cell immunoreceptor with Ig and ITIM domains (TIGIT), leukocyte-associated immunoglobulin-like receptor 1 (LAIRI), natural killer cell receptor 2B4 (2B4), and CD 160.

The present disclosure describes methods of producing mixed CAR cells from cells of a subject having cancer or a healthy donor. For example, the mixed CAR cells include conventional T cells (T cells expressing αβTCR), γδT lymphocytes (γδT cells), NKT, and/or NK cells. General information of CAR-engineered cells of each of these cells may be found in Rotolo, R. et al., CAR-Based Strategies beyond T Lymphocytes: Integrative Opportunities for Cancer Adoptive Immunotherapy. Int. J. Mol. Sci. 2019, 20, 2839, which is incorporated by reference in its entirety. CAR NKT cells effectively localized at the tumor site and presented strong antitumor activity without inducing GvHD. The adoptive transfer of expanded, activated autologous NK cells reported a limited clinical efficacy related to the inhibition by self-HLA molecules, while NK cells from an allogeneic source can hold the potential to be developed as a valid alternative approach. To minimize the occurrence of GvHD, allogeneic NK cells can be obtained from HLA-matched or haploidentical donors.

In embodiments, the method can include obtaining mixed cells including T cells and NK cells, and introducing a polynucleotide encoding a CAR into the mixed cells to obtain mixed CAR cells. For example, anti-CD56 magnetic beads can be implemented to select NK cells. In embodiments, the method can include obtaining T cells and NK cells, separately, introducing the polynucleotide into the T cells and NK cells to obtain CAR T cells and CAR NK cells, and mixing the CAR T cells and CAR NK cells to obtain the mixed CAR cells.

In embodiments, the method can include collecting peripheral blood mononuclear cells (PBMCs) from the subject and selecting CD3+ cells from the PBMCs. In certain embodiments, the PBMCs can be mixed with a group of antibodies to allow the group of antibodies to bind target cells. In these instances, the group of antibodies does not include CD3 antibodies. The target cells can be then removed from the PBMCs to obtain a solution containing the CD3+ cells. For example, the group of antibodies can include at least one of CD14, CD15, CD16, CD19, CD34, CD36, CD56, CD123, or CD235a. The CD3+ cells can then be cultured with anti-CD3 and anti-CD28 beads and introduced with a polynucleotide encoding a CAR to obtain CAR T cells. The method can further comprise obtaining CAR NK cells from the healthy donor. For example, the method can include collecting PBMCs from the healthy donor and selecting CD56+ cells from the PBMCs, which are then introduced with a polynucleotide encoding the CAR to obtain the CAR NK cells. The CAR T cells from a subject having cancer and CAR NK cells from a healthy donor can be mixed based on a predetermined ratio to obtain the mixed CAR cells, which are then infused into the subject. For example, the ratios between the CAR T cells and CAR NK cells comprises 1:10 to 1:1000,000 (e.g., 1:100, 1:1,000, 1:10,00, and 1:10,000). In embodiments, the method can include collecting peripheral blood mononuclear cells (PBMCs) from the subject and selecting CD3+ cells and CD56+ from the PBMCs, which are then introduced with a polynucleotide encoding the CAR to obtain both CAR T cells and CAR NK cells. These mixed CAR cells are then infused into the subject. In embodiments, whole blood cells or PMBCs can be introduced with a polynucleotide encoding CAR, and CAR T cells and/or CAR NK cells can be selected for the infusion. In embodiments, IL-15 can be added to the mixed cells for helping in vitro culturing of NK cells.

In embodiments, substantially whole blood can be used in the above embodiments. Substantially whole blood is the blood that is isolated from an individual(s), has not been subjected to a PBMC enrichment procedure, and is diluted by less than 50% with other solutions. For example, this dilution can be from the addition of an anti-coagulant as well as the addition of a volume of fluid comprising retroviral particles.

TABLE 2
Example mixture components

| | Mixture | Mixture | Mixture | Mixture |
|---|---|---|---|---|
| | component (I) | component (II) | component (III) | component (VI) |
| Activation | CD3/CD28 beads | CD3/CD28 beads | CD3/CD28 beads | CD3/CD28 beads |
| agents | | | | |
| Vectors | Vector encoding CD19 | Vector encoding CD19 | Vector encoding CD19 | Vector encoding CD19 |
| | CAR, NFAT-IL6 | CAR, NFAT-L6-2A-IFNγ | CAR, NFAT-IL6 | CAR, NFAT-IL6 |
| | Vector encoding CD19 | Vector encoding CD19 | Vector encoding solid | Vector encoding CD19 |
| | CAR, NFAT-IL12 | CAR, NFAT-IL12 | tumor, NFAT-IL12 | CAR, NFAT-IL12 |
| | Vector encoding CD19 | Vector encoding solid | Vector encoding solid | Vector encoding CD19 |
| | CAR, NFAT-IFNγ | tumor CAR | tumor, NFAT-IFNγ | CAR, NFAT-IFNγ |
| | Vector encoding solid | | Vector encoding solid | Vector encoding solid |
| | tumor CAR | | tumor CAR | tumor, NFAT-IL12 |
| | | | | Vector encoding solid |
| | | | | tumor, NFAT-IFNγ |
| | | | | Vector encoding solid |
| | | | | tumor CAR |

Embodiments relate to a system for immune therapy or causing a T cell response, the system comprising: a sample processing module configured to obtain T cells from a blood sample from a subject; a cell incubation module configured to activate T cell and introduce a vector into the T cells; and a cell infusion module configured to infuse at least a portion of the T to the subject, wherein: the sample processing module comprises: an input port configured to receive the blood sample from the subject; a sample separation module configured to: separate the T cells from remaining components of the blood sample, collecting the T cells; an output port configured to: flow the T cells to the cell incubation module, and wherein the sample separation module is configured to: contact the blood sample with a composition comprising T cell aggregation reagent comprising T cell recognizing moiety coupled to a magnetic particle or antigen recognizing moieties specifically binding undesired cellular components; apply simultaneously gravity sedimentation for sedimentation of T cells or undesired cells and a magnetic field gradient to the blood sample for immobilizing the magnetic particle; generate a pellet and a supernatant phase; and recover the desired cells from the supernatant phase or the pellet.

In embodiments, causing a T cell response includes stimulating a T cell response.

Embodiments relate to a system for immune therapy or causing a T cell response, the system comprising: a sample processing module configured to obtain blood cells from a blood sample from a subject; a cell incubation module configured to activate blood cells and/or introduce a vector into the blood cells; and a cell infusion module configured to infuse at least a portion of the transduced blood cells to the subject, wherein the blood cells comprising CD3+ cells.

In embodiments, the sample processing module is coupled to the cell incubation module that is coupled to the cell infusion module such that the blood cells flow through the system starting from the subject and ending back to the subject.

In embodiments, the sample processing module comprises: an input port configured to receive the blood sample from the subject; a sample separation module configured to: separate the blood cells from remaining components of the blood sample, collecting the blood cells; an output port configured to flow the blood cells to the cell incubation module, and flow the remaining components of the blood sample to the subject.

In embodiments, the sample separation module is configured to: contact the blood sample with a composition comprising a CD3+ aggregation reagent comprising CD3 recognizing moiety coupled to a magnetic particle or antigen recognizing moieties specifically binding undesired cellular components; apply gravity sedimentation for sedimentation of CD3+ cells or undesired cells and a magnetic field gradient to the blood sample for immobilizing the magnetic particle simultaneously; generate a pellet and a supernatant phase; and recover the desired cells from the supernatant phase or the pellet.

In embodiments, the system comprises a material composed of plastic. The plastic can comprise polystyrol, polystyrene, polyvinylchloride, polycarbonate, glass, polyacrylate, polyacrylamide, polymethylmethacrylate (PMMA), polyethylene terephthalate (PET), polytetrafluoroethylene (PTFE), thermoplastic polyurethane (TPU), silicone, polyethylene (PE), collagen, chitin, alginate, hyaluronic acid derivatives, polylactide (PLA), polyglycolide (PGA) and their copolymers, polystyrol, polystyrene, polycarbonate, polyacrylate, ceramics, glass materials, like hydroxyapatite (HA), and calcium phosphate, and compositions comprising one or more of the above-mentioned materials.

In embodiments, the system further comprises one or more sensors configured to detect the progress of separation of the blood sample from the subject, in particular by detecting the formation of layers of the blood sample, the change of pH value of the blood sample, and/or the change in temperature of the blood sample.

In embodiments, the cell incubation module comprises a rotating container configured to culture cells and/or grow cells. The rotating container is disposable and/or has been sterilized.

Embodiments relate to a method of immune therapy or causing a T cell response by implementing any system above, the method comprising: receiving peripheral blood of a subject; obtain the CD3+ cells from the peripheral blood; providing the remaining cells of the peripheral blood to the subject; obtaining vectors comprising a nucleic acid sequence encoding a CAR and one or more agents that activate T cells; mixing the vectors and the obtained CD3+ cells and incubating the mixture for a predetermined time to introduce the nucleic acid sequence into the CD3+ cells; replacing culturing media of the CD3+ cells with a solution suitable for infusion, and providing at least a portion of the CD3+ cells to the subject. In embodiments, the method is completed less than 1, 2, 3, 4, or 5 days.

Embodiments relate to an apparatus for sample processing or causing a T cell response, the apparatus comprising: an input port configured to receive blood sample from a subject; a sample separation module configured to isolate desired cells from the blood sample; and an output port configured to: flow the desired ells for further processing, and flow undesired cells and/or remaining components of the blood sample to the subject. In embodiments, the desired cells are blood cells (e.g., CD3+ cells), and the blood sample is peripheral blood.

In embodiments, the sample processing module can further comprise a unit configured to obtain human cells from the peripheral blood of the subject.

In embodiments, the unit is an apheresis device in which the blood of a subject is passed through an apparatus that separates out one particular constituent and returns the remainder to the circulation.

Embodiments relate to a method for sample processing or causing a T cell response by implementing any of the apparatus, system or device described herein, the method comprising: receiving a blood sample from a subject; isolating desired cells from the blood sample; flowing the desired ells for further processing; flowing undesired cells and/or remaining components of the blood sample to the subject; introducing a nucleic acid sequence encoding an antigen binding molecule into the desired cell; and administering an effective amount of a composition comprising the blood cells to the subject. In embodiments, the blood cells administered to the subject comprise CD3+ cells.

In embodiments, a vein-to-vein time is between 30 minutes and 1 hour (hr), 1 hr and 72 hours (hrs), 1 hr and 12 hrs, 1 hr and 24 hrs, 1 hr and 48 hrs, 12 hrs and 24 hrs, 12 hr and 48 hrs, or 48 and 72 hrs (including numbers between). In embodiments, the vein-to-vein time is between 30 minutes and 12 hrs or 12 hrs and 24 hrs. In embodiments, the vein-to-vein time is within 12 hrs or 24 hrs.

In embodiments, activation of the collected human cells and introduction of vectors into human cells are performed at the same time.

In embodiments, the vectors introduced into the human cells comprise a polynucleotide encoding a CAR targeting a WBC antigen (e.g., CD19, CD20, CD22, and BCMA), a second polynucleotide encoding a CAR targeting a solid tumor antigen (e.g., listed in Table 1), a third polynucleotide encoding IL-12, a fourth polynucleotide encoding IL-6, and/or a polynucleotide encoding IFNγ.

In embodiments, the vectors introduced into the human cells comprise a polynucleotide encoding a CAR targeting a WBC antigen (e.g., CD19, CD20, CD22, and BCMA), a second polynucleotide encoding a CAR targeting a solid tumor antigen (e.g., listed in Table 1), and a third polynucleotide encoding a cytokine. In embodiments, the vectors introduced into the human cells comprise a first vector comprising a polynucleotide encoding a CAR targeting a WBC antigen (e.g., CD19, CD20, CD22, and BCMA) and a second vector comprising a polynucleotide encoding a CAR targeting a solid tumor antigen (e.g., listed in Table 1), the first and second vectors being separate vectors.

In embodiments, the human cells infused into the subject comprise transduced T cells comprising a CAR targeting a WBC antigen, transduced T cells comprising a CAR targeting a solid tumor. In embodiments, the transduced T cells comprise a CAR targeting a WBC antigen that does not comprise a CAR targeting the solid tumor, and the transduced T cells comprising a CAR targeting a solid tumor do not comprise the CAR targeting the WBC antigen.

In embodiments, the human cells infused into the subject comprise one or more vectors listed in Table 2.

In embodiments, the CAR comprises an extracellular domain, a transmembrane domain, and an intracellular domain, and the extracellular domain binds an antigen. In embodiments, the intracellular domain comprises a co-stimulatory signaling region that comprises an intracellular domain of a co-stimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, and any combination thereof. In embodiments, the antigen is Epidermal growth factor receptor (EGFR), Variant III of the epidermal growth factor receptor (EGFRvIII), Human epidermal growth factor receptor 2 (HER2), Mesothelin (MSLN), Prostate-specific membrane antigen (PSMA), Carcinoembryonic antigen (CEA), Disialoganglioside 2 (GD2), Interleukin-13Ra2 (IL13Rα2), Glypican-3 (GPC3), Carbonic anhydrase IX (CAIX), L1 cell adhesion molecule (L1-CAM), Cancer antigen 125 (CA125), Cluster of differentiation 133 (CD133), Fibroblast activation protein (FAP), Cancer/testis antigen 1B (CTAG1B), Mucin 1 (MUC1), Folate receptor-α (FR-α), CD19, FZD10, TSHR, PRLR, Muc 17, GUCY2C, CD207, CD3, CD5, B-Cell Maturation Antigen (BCMA), or CD4.

In embodiments, the vectors described herein comprise a nucleic acid sequence encoding a binding molecule. In embodiments, the binding molecule is a CAR or TCR. In embodiments, the vector is a lentivirus.

Embodiments relate to a method of delivering a therapeutic protein to the peripheral blood system of a subject, the method comprising: administering to the peripheral blood system of the subject a viral vector comprising a nucleic acid sequence encoding an antigen binding molecule such that cells, organ or tissue of the peripheral blood system express the antigen binding molecule in the subject.

In embodiments, the nucleic acid encoding the therapeutic protein described herein has reduced cytosine-guanine dinucleotide (CpG) compared to a non-CpG reduced nucleic acid encoding the therapeutic protein.

In embodiments, the nucleic acid encoding the therapeutic protein described herein has reduced cytosine-guanine dinucleotide (CpG) compared to a wild-type nucleic acid encoding the therapeutic protein.

Embodiments relate to a method of treating cancer in a subject, the method comprising: administering to the peripheral blood system of the subject a viral vector comprising a nucleic acid sequence encoding a chimeric antigen receptor such that cells, organ or tissue of the peripheral blood system express the antigen binding molecule of the subject; and monitoring T cell response derived from the expression of antigen binding molecule in the subject.

Embodiments relate to a method of in vivo expression of antigen binding molecule in lymphocytes of a subject, the method comprising: administering to the peripheral blood system of the subject a viral vector comprising a nucleic acid sequence encoding a chimeric antigen receptor such that cells, organ or tissue of the peripheral blood system express the antigen binding molecule of the subject.

In embodiments, the viral vector is an rAAV particle comprising an AAV capsid protein and a vector comprising the nucleic acid encoding antigen binding molecule inserted between a pair of AAV inverted terminal repeats (ITRs) in a manner effective to infect the cells, organ, or tissue of the peripheral system in the subject such that the cells, organ or tissue express antigen binding molecule.

In embodiments, the vector further comprises one or more of an intron, an expression control element, a filler polynucleotide sequence and/or poly A signal, or a combination thereof.

In embodiments, the pair of ITRs are derived from or comprises a sequence of any of AAV1, AAV2, AAV3, AAV4, AAV5, AAV6, AAV7, AAV8, AAV9, AAV10, AAV11, AAV 12, LK01, LK02, LK03, AAV 4-1, AAV-218 ITRs, or a mixture thereof.

In embodiments, the AAV capsid protein is derived from or comprise a sequence any of: AAV1, AAV2, AAV3, AAV4, AAV5, AAV6, AAV7, AAV8, AAV9, AAV10, AAV11, AAV 12, LK01, LK02, LK03, AAV 4-1, AAV-218 ITRs, or a mixture thereof.

In embodiments, the rAAV particles are administered at a dose of about 5×1011 vector genomes per kilogram (vg/kg) of the subject.

In embodiments, the subject does not develop a substantial immune response against the rAAV particle. In embodiments, the subject does not develop a substantial humoral immune response against the rAAV particle. In embodiments, the subject does not develop a substantial immune response against the rAAV particle for at least 1, 2, 3, 4, 5, 6, 7, 8, 9, 10, 11, 12, 13, or 14 continuous days, weeks or months.

In embodiments, the subject does not develop a detectable immune response against the rAAV particle. In embodiments, the subject does not produce an immune response against the therapeutic protein and/or the rAAV particle sufficient to block the therapeutic effect in the subject for at least 1, 2, 3, 4, 5, 6, 7, 8, 9, 10, 11, 12, 13 or 14 continuous days, weeks or months.

In embodiments, the administering comprises infusion or injection into the systemic circulation of the subject. In embodiments, the administering comprises intravenous or intra-arterial infusion or injection into the systemic circulation of the subject.

In embodiments, the viral vector further comprises a nucleic acid encoding IL-12, and anti-tumor activities of the modification are enhanced as compared to a subject that is administered with the viral vector without the nucleic acid encoding IL-12, and expression of IL-12 is regulated by NFAT promoter.

In embodiments, the method further comprises administering the subject with an effective amount of CD28 and CD3 agonists and/or protamine sulfate, wherein transduction ratios and anti-tumor activities are enhanced as compared with a subject that is administered with the viral vector without the arsonists and protamine sulfate.

Embodiments relate to a modified cell generated or processed by any suitable preceding method, apparatus, device, or systems.

In embodiments, the modified cell comprises an antigen binding molecule, wherein expression and/or function of one or more molecules in the modified cell has been enhanced or reduced (including eliminated), and wherein the one or more molecules comprising at least one of G-CSF, GM-CSF, and a derivative of G-CSF or GM-CSF.

Embodiments relate to a kit comprising an effective amount of vector-free nucleic acids encoding at least one of G-CSF, GM-CSF, and a derivative of G-CSF or GM-CSF to render a population of immune cells specific for a tumor antigen expressed on the surface of the cells of a subject.

Embodiments relate to a method or use of polynucleotide, the method comprising: providing a viral particle (e.g., AAV, lentivirus or their variants) comprising a vector genome, the vector genome comprising the polynucleotide encoding G-CSF, GM-CSF, and a derivative of G-CSF or GM-CSF and a polynucleotide encoding an antigen binding molecule, the polynucleotide operably linked to an expression control element conferring transcription of the polynucleotides; and administering an amount of the viral particle to a subject such that the polynucleotide is expressed in the subject, wherein the one or more molecules are overexpressed in cancer cells, associated with recruitment of immune cells, and/or associated with autoimmunity.

In embodiments, the AAV preparation can include AAV vector particles, empty capsids, and host cell impurities, thereby providing an AAV product substantially free of AAV empty capsids.

Embodiments relate to a method of eliciting or enhancing T cell response, treating a subject in need thereof or enhancing cancer treatment thereof, the method comprising administering an effective amount of the composition of or the kit described herein to the subject.

In embodiments, the enhanced expression and/or function of the one or more molecules is implemented by introducing a nucleic acid encoding the one or more molecules and/or the binding molecule, which is present in the modified cell in a recombinant DNA construct, in an mRNA, or in a viral vector.

In embodiments, the nucleic acid is an mRNA, which is not integrated into the genome of the modified cell. In embodiments, the nucleic acid is associated with an oxygen-sensitive polypeptide domain. In embodiments, the oxygen-sensitive polypeptide domain comprises HIF VHL binding domain.

In embodiments, the nucleic acid sequence is regulated by a promoter comprising a binding site for a transcription modulator that modulates the expression and/or secretion of the therapeutic agent in the cell. In embodiments, the transcription modulator is or includes Hif1a, NFAT, FOXP3, and/or NFkB.

In embodiments, the one or more molecules comprise at least one of G-CSF or GM-CSF, or a combination thereof. In embodiments, one or more molecules comprise at least one of a receptor of G-CSF or GM-CSF, or a combination thereof. In embodiments, one or more molecules comprise at least one of IL-33, IL-1β, TNFα, MALP-2, IL1, and IL17.

In embodiments, the modified cell comprises the antigen binding molecule, wherein the antigen binding molecule is a chimeric antigen receptor (CAR) comprising an antigen-binding domain, a transmembrane domain, and an intracellular signaling domain.

In embodiments, the antigen-binding domain binds to a tumor antigen is selected from a group consisting of: TSHR, CD19, CD123, CD22, CD30, CD171, CS-1, CLL-1, CD33, EGFRvIII, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-11Ra, PSCA, PRSS21, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6, E7, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, and IGLL1.

In embodiments, the intracellular signaling domain comprises a co-stimulatory signaling domain, or a primary signaling domain and a co-stimulatory signaling domain, wherein the co-stimulatory signaling domain comprises a functional signaling domain of a protein selected from the group consisting of CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D. In embodiments, the modified cell comprises the antigen binding molecule, the antigen binding molecule is a modified TCR. In embodiments, the TCR is derived from spontaneously occurring tumor-specific T cells in patients. In embodiments, the TCR binds to a tumor antigen. In embodiments, the tumor antigen comprises CEA, gp100, MART-1, p53, MAGE-A3, or NY-ESO-1. In embodiments, the TCR comprises TCRγ and TCRδ Chains or TCRα and TCRβ chains, or a combination thereof.

In embodiments, the modified cell is derived from an immune cell (e.g., a population of immune effector cells). In embodiments, the immune cell is a T cell or an NK cell. In embodiments, the immune effector cell is a T cell. In embodiments, the T cell is a CD4+ T cell, a CD8+ T cell, or a combination thereof. In embodiments, the cell is a human cell.

In embodiments, the modified cell comprises a nucleic acid sequence encoding a binding molecule and a dominant negative form of an inhibitory immune checkpoint molecule or a receptor thereof. In embodiments, the inhibitory immune checkpoint molecule is selected from the group consisting of programmed death 1 (PD-1), cytotoxic T lymphocyte antigen-4 (CTLA-4), B- and T-lymphocyte attenuator (BTLA), T cell immunoglobulin mucin-3 (TIM-3), lymphocyte-activation protein 3 (LAG-3), T cell immunoreceptor with Ig and ITIM domains (TIGIT), leukocyte-associated immunoglobulin-like receptor 1 (LAIRI), natural killer cell receptor 2B4 (2B4), and CD 160.

In embodiments, the inhibitory immune checkpoint molecule is modified PD-1. In embodiments, the modified PD-1 lacks a functional PD-1 intracellular domain for PD-1 signal transduction, interferes with a pathway between PD-1 of a human T cell of the human cells and PD-L1 of a certain cell, comprises or is a PD-1 extracellular domain or a PD-1 transmembrane domain, or a combination thereof, or a modified PD-1 intracellular domain comprising a substitution or deletion as compared to a wild-type PD-1 intracellular domain, or comprises or is a soluble receptor comprising a PD-1 extracellular domain that binds to PD-L1 of a certain cell.

In embodiments, the modified cell is engineered to express and secrete a therapeutic agent such as a cytokine. In embodiments, the cytokine is or comprises IL-6 or IFN-γ, or a combination thereof. In embodiments, the cytokine is or comprises IL-15 or IL-12, or a combination thereof.

In embodiments, the modified cell is engineered to express or secrete a small protein such as a recombinant or native cytokine. In embodiments, the small protein is or comprises IL-12, IL-6, or IFN-γ.

In embodiments, the modified cell is derived from a healthy donor or the subject having cancer.

In embodiments, the modified cell has a reduced expression of the endogenous T cell receptor alpha constant (TRAC) gene.

In embodiments, the modified cell comprises a first CAR binding an antigen of a white blood cell and a second CAR binding a solid tumor antigen. In embodiments, the modified cell comprises a bispecific CAR binding a white blood antigen and a solid tumor antigen.

Embodiments relate to a pharmaceutical composition comprising a population of the modified cells and a population of additional modified cells, wherein the modified cells bind a first antigen, and the additional modified cells bind a second antigen, which is different from the first antigen. In embodiments, the first antigen is a WBC antigen, and the second antigen is a solid tumor antigen. In embodiments, the second antigen is a WBC antigen, and the first antigen is a solid tumor antigen.

In embodiments, the WBC antigen is or comprises CD19, CD22, CD20, BCMA, CD5, CD7, CD2, CD16, CD56, CD30, CD14, CD68, CD11b, CD18, CD169, CD1c, CD33, CD38, CD138, or CD13. In embodiments, the WBC antigen is or comprises CD19, CD20, CD22, or BCMA.

In embodiments, the solid tumor antigen comprises tMUC 1, PRLR, CLCA1, MUC12, GUCY2C, GPR35, CR1L, MUC 17, TMPRSS11B, MUC21, TMPRSS11E, CD207, SLC30A8, CFC1, SLC12A3, SSTR1, GPR27, FZD10, TSHR, SIGLEC15, SLC6A3, KISS1R, CLDN18.2, QRFPR, GPR119, CLDN6, UPK2, ADAM12, SLC45A3, ACPP, MUC21, MUC16, MS4A12, ALPP, CEA, EphA2, FAP, GPC3, IL13-Ra2, Mesothelin, PSMA, ROR1, VEGFR-II, GD2, FR-α, ErbB2, EpCAM, EGFRvIII, B7-H3, or EGFR. In embodiments, the solid tumor antigen comprises tumor-associated MUC1, ACPP, TSHR, GUCY2C, UPK2, CLDN18.2, PSMA, DPEP3, CXCR5, B7-H3, MUC16, SIGLEC-15, CLDN6, Muc17, PRLR, or FZD10.

Embodiments relate to a method of eliciting or enhancing T cell response, treating a subject in need thereof or enhancing cancer treatment thereof, the method comprising administering an effective amount of the pharmaceutical composition comprising modified T cells. In embodiments, the modified T cells comprise a nucleic acid encoding hTERT, SV40LT, or a combination thereof. In embodiments, the modified T cells are more proliferable than T cells without the nucleic acid encoding hTERT, SV40LT, or a combination thereof. In embodiments, the proliferable T cells retain functions of normal T cells/CAR T cells, for example, cell therapy functions.

In embodiments, integration of the nucleic acid encoding hTERT, the nucleic acid encoding SV40LT, or a combination thereof includes genomic integration of the nucleic acid encoding hTERT, a nucleic acid encoding SV40LT, or a combination thereof and constitutive expression of hTERT, SV40LT, or a combination thereof. In embodiments, expression of hTERT, SV40LT, or a combination thereof, is regulated by an inducible expression system such as a rtTA-TRE system.

In embodiments, the modified T cells comprise a CAR and the nucleic acid encoding hTERT, nucleic acid encoding SVOLT, or a combination thereof, and the modified T cells are cultured in the presence of an agent that is recognized by the extracellular domain of the CAR.

In embodiments, the modified T cells comprise a nucleic acid sequence encoding a suicide gene such as an HSV-TK system.

In embodiments, the modified T cells have reduced graft-versus-host disease (GVHD) response in a bioincompatible human recipient as compared to the GVHD response of the primary human T cell.

In embodiments, the modified T cells have reduced expression of the endogenous TRAC gene.

In embodiments, the blood sample from the subject includes peripheral blood, which then is activated by a T cell activator (e.g., beads conjugated with anti-CD3 and beads conjugated with anti-CD28) and/or introduced with a polynucleotide encoding a binding molecule (e.g., CAR or TCR).

Embodiments relate to a method of enhancing in vivo transduction of T cells or enhancing a gene therapy, the method comprising: administering to a subject having a form of cancer an effective amount of a pharmaceutical composition comprising a viral vector comprising a nucleic acid encoding a CAR or TCR and one or more compounds, the one or more compounds comprising a T cell activator and/or a transduction adjuvant; and allowing the viral vector to be introduced into the T cells of the subject, wherein a transduction rate of the nucleic acid into the T cells is greater than a transduction rate of nucleic acid into T cells of a subject that is administered with an effective amount of a pharmaceutical composition comprising the viral vector without the one or more compounds.

Embodiments relate to a method of performing or enhancing a gene therapy, the method comprising: administering to a subject having a form of cancer with an effective amount of a pharmaceutical composition comprising a viral vector comprising a nucleic acid encoding a CAR or TCR and one or more compounds, the one or more compounds comprising a T cell activator and a transduction adjuvant; and allowing the viral vector to be introduced into the T cells of the subject.

In embodiments, the methods related to gene therapies herein can be combined with techniques associated with CoupledCAR® described in PCT Publication Nos: WO2020106843 and WO2020146743, which are incorporated by their entirety. In embodiments, the viral vector(s) can include a nucleic acid encoding a CAR targeting a WBC antigen (e.g., CD19 and BCMA) and a nucleic acid encoding a CAR targeting a solid tumor antigen (e.g., GCC and TSHR). In embodiments, the viral vector(s) can include (1) a nucleic acid encoding a CAR targeting a WBC antigen (e.g., CD19 and BCMA) and IL-6, a nucleic acid encoding the CAR targeting the WBC antigen and IL-12, or a nucleic acid encoding the CAR targeting the WBC antigen and IFN-γ, or a combination thereof, and (2) a nucleic acid encoding a CAR targeting a solid tumor antigen (e.g., GCC and TSHR).

In embodiments, the intracellular domain of the CAR can comprise a co-stimulatory domain and a domain associated with the signaling of IL-2R or an exogenous JAK-binding motif (e.g., JAK-STAT domain). In embodiments, the term “exogenous association motif” means any association motif that is recombinantly introduced into a domain, for example, an intracellular signaling domain such as a cytoplasmic domain of an interleukin receptor chain, a cytoplasmic co-stimulatory domain, or a CD34 intracellular signaling domain, but that does not exist natively in the domain or at the specific location in the domain. For example, an exogenous JAK-binding motif can be inserted into an intracellular signaling domain, such as a cytoplasmic domain of an interleukin receptor chain. The “JAK-binding motif” used herein refers to a BOX-1 motif that allows for tyrosine kinase JAK association, for example, JAK1. The JAK-binding motif can be, for example, amino acid numbers 278 to 286 of NCBI RefSeq: NP_000869.1. In this instance, a “domain” means one region in a polypeptide, for example, which is folded into a particular structure independently of other regions and/or has a particular function. The domain can, for example, be the cytoplasmic portion of a molecule or a part thereof. As used herein, the “cytoplasmic domain” of a molecule can refer to the full cytoplasmic domain or a part thereof that induces an intracellular signal when activated.

The term “variant” refers to a molecule comprising a substitution, deletion, or addition of one or a few to a plurality of amino acids and includes particularly conservatively substituted molecules, provided that the variant substantially retains the same function as the original sequence. For example, IL receptor variants can comprise substitutions, deletions, or additions outside the JAK-binding motif and the STAT association motif. Thus, an IL receptor chain variant can comprise up to 50, up to 40, up to 30, up to 20, or up to 10 amino acid deletion and/or conservative substitutions in a region outside of the JAK-binding and STAT association motifs. Similarly, variants of other molecules can comprise up to 50, up to 40, up to 30, up to 20, or up to 10 amino acid deletion and/or conservative substitutions, in a region outside of a region identified specifically herein. As used herein, the phrase “wherein the intracellular segment comprises an endogenous or exogenous JAK-binding motif and a STAT5 association motif” includes the intracellular segment comprising more than one cytoplasmic domain, and the JAK binding motif and the STAT5 association motif can be in the same cytoplasmic domain or can be in separate cytoplasmic domains. In embodiments, the viral vector(s) can include a nucleic acid encoding IL2R such that white blood cells (e.g., T cells) can overexpress IL2R, enhancing the expansion of the white blood cells.

In embodiments, the transduction adjuvant is or comprises protamine sulfate (PS), optionally from 1 micro g/ml to 50 micro g/ml or from about 1 micro g/ml to about 50 micro g/ml protamine sulfate; a fibronectin-derived transduction adjuvant; and/or RetroNectin.

In embodiments, the T cell activator comprises anti-CD3 and/or anti-CD28 antibodies.

In embodiments, the one or more compounds comprise antibodies or agonists binding CD3 and/or CD28 and PS.

In embodiments, the viral vector comprises a nucleic acid encoding IL-12, wherein anti-tumor activities in the subject are greater than in a subject that is administered with a viral vector comprising the nucleic acid encoding the CAR in the absence of the nucleic acid encoding IL-12.

Embodiments relate to a method for (1) causing T cell response in a subject, (2) expanding T cells in a subject, (3) inhibiting the growth of tumor cells in a subject, (4) genetically modifying lymphocytes (e.g., NK or T cells), and/or (5) treating tumors in a subject, the method comprising: administering to the subject an effective amount of a recombinant virus comprising a nucleic acid encoding a chimeric antigen receptor (CAR). In embodiments, the recombinant virus further comprises a nucleic acid encoding a therapeutic agent (e.g., cytokines). In embodiments, the subject has liquid cancer, such as blood cancer (Non-Hodgkin's Lymphoma (NHL)). In embodiments, the CAR binds a WBC antigen (e.g., CD19, CD20, BCMA). In embodiments, the method further comprises administering a therapeutic agent such as IL-12.

Embodiments relate to a method for (1) causing T cell response in a subject, (2) expanding T cells in a subject, (3) inhibiting the growth of tumor cells in a subject, (4) genetically modifying lymphocytes, and/or (5) treating tumors in a subject, the method comprising: administering to the subject an effective amount of a recombinant virus carrying a nucleic acid sequence encoding a first CAR binding a first antigen and a second CAR binding a second antigen.

Embodiments relate to a method for (1) causing T cell response in a subject, (2) expanding T cells in a subject, (3) inhibiting the growth of tumor cells in a subject, (4) genetically modifying lymphocytes, and/or (5) treating tumors in a subject, the method comprising: administering to the subject an effective amount of a first recombinant virus carrying a nucleic acid sequence encoding a first CAR binding a first antigen and a second recombinant virus carrying a nucleic acid sequence encoding a second CAR binding a second antigen. In embodiments, the first antigen comprises a cell surface molecule of a WBC, a tumor antigen, or a solid tumor antigen.

In embodiments, the WBC is a granulocyte, a monocyte, or a lymphocyte. In embodiments, the WBC is a B cell. In embodiments, the cell surface molecule of the WBC is CD19, CD22, CD20, BCMA, CD5, CD7, CD2, CD16, CD56, CD30, CD14, CD68, CD11b, CD18, CD169, CD1c, CD33, CD38, CD138, or CD13. In embodiments, the cell surface molecule of the WBC is CD19, CD20, CD22, or BCMA. In embodiments, the cell surface molecule of the WBC is CD19 or BCMA.

In embodiments, the tumor antigen comprises a solid tumor antigen. In embodiments, the solid tumor antigen comprises tumor associated MUC1 (tMUC1), PRLR, CLCA1, MUC12, GUCY2C, GPR35, CR1L, MUC 17, TMPRSS11B, MUC21, TMPRSS11E, CD207, SLC30A8, CFC1, SLC12A3, SSTR1, GPR27, FZD10, TSHR, SIGLEC15, SLC6A3, KISS1R, CLDN18.2, QRFPR, GPR119, CLDN6, UPK2, ADAM12, SLC45A3, ACPP, MUC21, MUC16, MS4A12, ALPP, CEA, EphA2, FAP, GPC3, IL13-Ra2, Mesothelin, PSMA, ROR1, VEGFR-II, GD2, FR-α, ErbB2, EpCAM, EGFRvIII, B7-H3, or EGFR. In embodiments, the solid tumor antigen comprises tMUC1, ACPP, TSHR, GUCY2C, UPK2, CLDN18.2, PSMA, DPEP3, CXCR5, B7-H3, MUC16, SIGLEC-15, CLDN6, Muc17, PRLR, or FZD10. In embodiments, the solid tumor antigen comprises tMUC1, ACPP, TSHR, GUCY2C, UPK2, or CLDN18.2.

In embodiments, the CAR comprises an antigen binding domain, a transmembrane domain, a co-stimulatory domain, and a CD3 zeta domain. In embodiments, the co-stimulatory domain comprises the intracellular domain of CD27, CD28, 4-1BB, OX40, CD30, CD40, PD-1, ICOS, lymphocyte function-associated antigen-1 (LFA-1), CD2, CD7, LIGHT, NKG2C, B7-H3, a ligand that binds CD83, or a combination thereof.

In embodiments, the first CAR comprises an scFv binding CD19, an intracellular domain of 4-1BB or CD28, and CD3 zeta domain, and the second CAR comprises an scFv binding tMUC1, ACPP, TSHR, GUCY2C, or CLDN18.2., an intracellular domain of 4-1BB or CD28, and CD3 zeta domain.

In embodiments, the nucleic acid sequence further encodes a dominant negative form of PD-1.

In embodiments, the nucleic acid sequence further encodes a therapeutic agent. In embodiments, the therapeutic agent comprises a cytokine. In embodiments, the cytokine is IL6 and/or INF-γ. In embodiments, the cytokine is at least one of IL6, IL12, IL-15, IL-7, TNF-α, or IFN-γ.

In embodiments, the nucleic acid sequence is under the control of a promoter sequence which expresses the product of the nucleic acid sequence in lymphocytes.

In embodiments, the recombinant virus is Adenovirus, Adeno-Associated Virus (AAV), Retrovirus (e.g., MMLV), or Lentivirus (e.g., HIV-1, FIV, SIV). In embodiments, the recombinant virus is Lentivirus. In embodiments, the recombinant virus is AAV. In embodiments, the effective amount comprises 1×109 to 2×1012 rAAV.

In embodiments, the promoter is a cell-specific promoter. In embodiments, the promoter is the chicken beta-actin promoter/CMV enhancer.

Embodiments relate to a pharmaceutical composition comprising at least one of the recombinant viruses of any suitable preceding embodiments.

Embodiments relate to a pharmaceutical composition of (1) causing T cell response in a subject, (2) expanding T cells in a subject, (3) inhibiting growth of tumor cells in a subject, (4) genetically modifying lymphocytes, (5) treating tumors in a subject, and/or (6) enhancing T cell response, T cell expansion in vivo, inhibition growth of tumor cells, and/or treatment of the tumor, the pharmaceutical composition comprising: an effective amount of one or more recombinant viruses carrying a nucleic acid sequence encoding the first CAR of any preceding suitable embodiments binding a first antigen and the second CAR of any preceding suitable embodiments binding a second antigen.

Embodiments relate to a method for (1) causing T cell response in a subject, (2) expanding T cells in a subject, (3) inhibiting the growth of tumor cells in a subject, (4) genetically modifying lymphocytes, and/or (5) treating tumors in a subject, the method comprising: administering to the subject an effective amount of the composition described above. In embodiments, the method further comprises: administering to the subject an effective amount of an agent that activates T cells to allow the introduction of the recombinant virus(es).

In embodiments, the agent comprises a T cell activator (e.g., beads conjugated with anti-CD3 and beads conjugated with anti-CD28). In embodiments, the agent comprises Transact™.

In embodiments, the recombinant virus(es) are in the form of a viral particle (e.g., replication-incompetent recombinant retroviral particle) that comprises a membrane-bound T cell activation element on the surface of the particle (more information about the replication-incompetent recombinant retroviral particle can be found in PCT Patent Publication No: WO2018009923 and WO2018161064, which are incorporated herein in its entirety).

In embodiments, the membrane-bound T cell activation element comprises or is anti-CD3 scFv.

In embodiments, viral particle further comprises a membrane-bound polypeptide capable of binding to CD28.

In embodiments, the composition comprises CD3 and CD28 agonists.

In embodiments, T cell response, T cell expansion in vivo, inhibition growth of tumor cells, a level of modification (e.g., expression ratio) in vivo, and/or treatment of the tumor are enhanced as compared with a subject that is administered with the composition without CD3 and CD28 agonists. In embodiments, the composition comprises an effective amount of protamine sulfate.

In embodiments, T cell response, T cell expansion in vivo, inhibition growth of tumor cells, a level of modification (e.g., expression ratio) in vivo, and/or treatment of the tumor are enhanced as compared with a subject that is administered with the composition without the protamine sulfate.

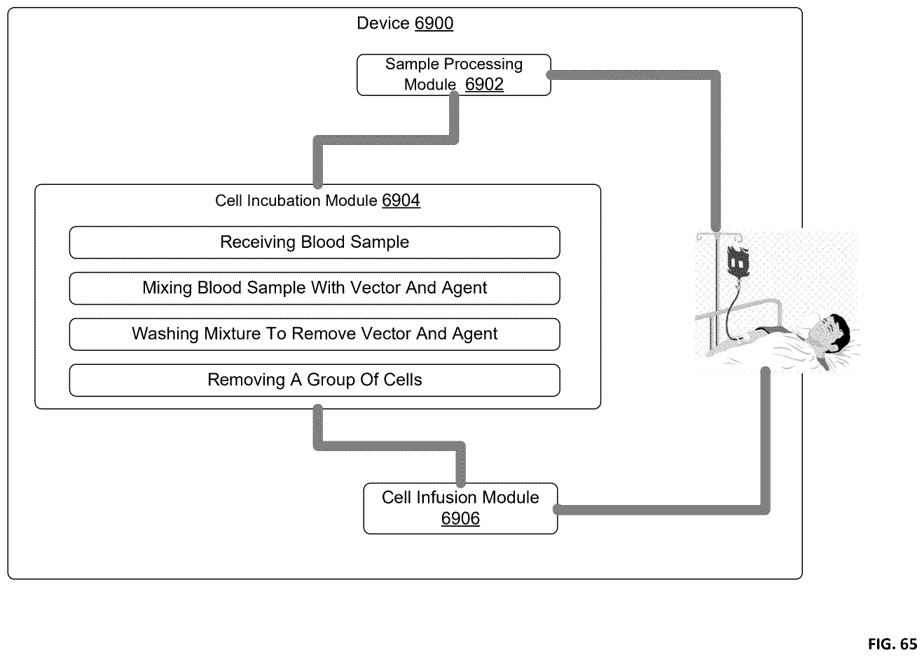

Embodiments relate to a device for immune therapy or causing a T cell response. An example of the device is illustrated in FIG. 65. The device 6900 comprises a sample processing module 6902, a cell incubation module 6904, and a cell infusion module 6906. In embodiments, the sample processing module is configured to a blood sample from a subject, the blood sample comprising T cells. The cell incubation module 6904 is configured to receive the blood sample, mix the blood sample with one or more vectors and an agent that activates T cells to introduce the one or more vectors into cells of the blood sample, and wash the blood sample to remove the one or more nontransduced vectors. The cell infusion module 6906 is configured to infuse at least a portion of the blood sample to the subject. In embodiments, the sample processing module is coupled to the cell incubation module that is coupled to the cell infusion module such that the portion of the blood sample flow through the device starting from the subject and ending back to the subject.

In embodiments, the blood sample comprises substantial whole blood, and the portion of the blood sample comprises at least two of CD3+ cells, NK cells, myeloid cells, and Neutrophil. In embodiments, the myeloid cells can include a vector encoding IL-12 such that the myeloid cells can overexpress IL-12.

In embodiments, the cell incubation module is further configured to remove one or more blood cells from the blood sample, and one or more blood cells comprise B cells. In embodiments, the cell incubation module is further configured to remove one or more blood cells from the blood sample using a bead conjugated with an antibody against a B cell marker.

In embodiments, the blood sample comprises PBMCs.

In embodiments, the agent comprises a T cell activator. In embodiments, the agent comprises a bead conjugated with anti-CD3 and a bead conjugated with anti-CD28. In embodiments, the agent comprises Transact™.

In embodiments, one or more vectors are in the form of a viral particle that comprises a membrane-bound T cell activation element on the surface of the viral particle. In embodiments, the membrane-bound T cell activation element comprises or is anti-CD3. In embodiments, the viral particle further comprises a membrane-bound polypeptide capable of binding to CD28.

In embodiments, the device comprises a material composed of plastic. The plastics can comprise polystyrol, polystyrene, polyvinylchloride, polycarbonate, glass, polyacrylate, polyacrylamide, polymethylmethacrylate (PMMA), polyethylene terephthalate (PET), polytetrafluoroethylene (PTFE), thermoplastic polyurethane (TPU), silicone, polyethylene (PE), collagen, chitin, alginate, hyaluronic acid derivatives, polylactide (PLA), polyglycolide (PGA) and their copolymers, polystyrol, polystyrene, polycarbonate, polyacrylate, ceramics, glass materials, like hydroxyapatite (HA), and calcium phosphate.

In embodiments, the device further comprises one or more sensors configured to detect the progress of separation of the blood sample, in particular by detecting the formation of layers of the blood sample, a change of pH value of the blood sample, and/or a change in temperature of the blood sample.

In embodiments, the cell incubation module comprises a rotating container configured to culture cells and/or grow cells. The rotating container is disposable and/or has been sterilized.

In embodiments, a vein-to-vein time is between 30 minutes and 1 hour (hr), 1 hr and 72 hours (hrs), 1 hr and 12 hrs, 1 hr and 24 hrs, 1 hr and 48 hrs, 12 hrs and 24 hrs, 12 hr and 48 hrs, or 48 and 72 hrs (including numbers between). In embodiments, the vein-to-vein time is between 30 minutes and 12 hrs or 12 hrs and 24 hours. In embodiments, the vein-to-vein time is within 12 hrs or 24 hrs.

In embodiments, one or more vectors comprise a polynucleotide encoding a CAR targeting a WBC antigen and a polynucleotide encoding a CAR targeting a solid tumor antigen.

In embodiments, the one or more vectors further comprise a polynucleotide encoding IL-12, a polynucleotide encoding IL-6, and/or a polynucleotide encoding IFNγ.

In embodiments, the one or more vectors comprise a polynucleotide encoding a CAR targeting a solid tumor antigen, and the solid tumor antigen is a tumor associated MUC1 (tMUC1), PRLR, CLCA1, MUC12, GUCY2C, GPR35, CR1L, MUC 17, TMPRSS11B, MUC21, TMPRSS11E, CD207, SLC30A8, CFC1, SLC12A3, SSTR1, GPR27, FZD10, TSHR, SIGLEC15, SLC6A3, KISS1R, CLDN18.2, QRFPR, GPR119, CLDN6, UPK2, ADAM12, SLC45A3, ACPP, MUC21, MUC16, MS4A12, ALPP, CEA, EphA2, FAP, GPC3, IL13-Ra2, Mesothelin, PSMA, ROR1, VEGFR-II, GD2, FR-α, ErbB2, EpCAM, EGFRvIII, B7-H3, or EGFR.

In embodiments, the CAR comprises an antigen binding domain, a transmembrane domain, a co-stimulatory domain, and a CD3 zeta domain. In embodiments, the co-stimulatory domain comprises an intracellular domain of CD27, CD28, 4-1BB, OX40, CD30, CD40, PD-1, ICOS, lymphocyte function-associated antigen-1 (LFA-1), CD2, CD7, LIGHT, NKG2C, B7-H3, a ligand that binds CD83, or a combination thereof.

Embodiments relate to a method of CAR T therapy, the method comprising: obtaining a blood sample from a subject, the blood sample comprising substantial whole blood; removing one or more cells from the blood sample to obtain a group of cells comprising at least CD3+ cells, NK cells, myeloid cells, and Neutrophils, the one or more cells comprising B cells; mixing the group of cells with one or more vectors and an agent that activates T cells to introduce the one or more vectors into the group of cells; and infusing at least a portion of the group of cells to the subject.

Embodiments relate to a method of CAR T therapy, the method comprising: obtaining a blood sample from a subject, the blood sample comprising substantial whole blood; mixing the blood sample with one or more vectors and an agent that activates T cells to introduce the one or more vectors into cells of the blood sample; removing one or more cells from the blood sample to obtain a group of cells comprising at least CD3+ cells, NK cells, myeloid cells, and Neutrophils, the one or more cells comprising B cells; and infuse at least a portion of the blood sample to the subject.

In embodiments, a vein-to-vein time is between 30 minutes and 1 hour (hr), 1 hr and 72 hours (hrs), 1 hr and 12 hrs, 1 hr and 24 hrs, 1 hr and 48 hrs, 12 hrs and 24 hrs, 12 hr and 48 hrs, or 48 and 72 hrs (including numbers between). In embodiments, the vein-to-vein time is between 30 minutes and 12 hrs or 12 hrs and 24 hrs. In embodiments, the vein-to-vein time is within 12 hrs or 24 hrs.

In embodiments, one or more vectors comprise a polynucleotide encoding a chimeric antigen receptor (CAR) targeting a WBC antigen and a polynucleotide encoding a CAR targeting a solid tumor antigen.

In embodiments, the one or more vectors further comprise a polynucleotide encoding IL-12, a polynucleotide encoding IL-6, and/or a polynucleotide encoding IFNγ.

In embodiments, the gene and/or cell therapy described herein can be implemented by fusosome techniques. For example, the one or more vectors can be incorporated into fusosomes, which can be used as a vehicle for the delivery of nucleic acids in CAR T therapy. A fusosome comprises a bilayer of amphipathic lipids enclosing a cavity containing a fusogen. The fusosome can be a membrane preparation obtained from a cell. Examples of fusosome include an extracellular vesicle, a microvesicle, a nanovesicle, an exosome, an apoptotic body (from apoptotic cells), a microparticle (obtained from, for example, platelets), an ectosome (obtained from, for example, neutrophiles and monocytes in serum), a prostatosome (obtained from prostate cancer cells), a cardiosome (obtained from cardiac cells), or any combination thereof. Fusosomes can be released naturally from a cell or can be obtained from cells treated to enhance their formation. Fusosomes can also comprise synthetic lipids.

The fusogen enclosed in the cavity of the fusosome can be a fusogen encoded by an endogenous retroviral envelope gene or a fusogen encoded by a pseudotyped vector. Examples of fusogen include a protein, a glycoprotein, a lipid, a small molecule, a virus, or a nucleic acid. In embodiments, the nucleic acid can encode an antibody binding molecule, such as a CAR molecule. The nucleic acid can comprise viral nucleic acid, such as retroviral nucleic acid, for example, a lentiviral nucleic acid. The nucleic acid can comprise nucleic acid of AAV. In embodiments, the fusogen includes therapeutic protein, antibody binding molecule, or a non-mammalian protein, such as a viral protein.

Fusosomes have the desired properties for facilitating the delivery of agents including nucleic acids for gene therapy to a target cell. The agents are the fusogen described herein. In embodiments, the fusosome containing the fusogen can fuse with a target cell. In embodiments, the fusosome. In embodiments, the fusosome containing the fusogen can be administered to a subject in need thereof for treatment of disease, as described herein.

In embodiments, the fusosome described herein comprises one or more fusogens, such as one or more nucleic acids encoding one or more antibody binding molecules, such as CAR molecules. In embodiments, the one or more nucleic acids comprise one or more vectors including the nucleic acid encoding a CAR binding a WBC antigen and the nucleic acid encoding a CAR binding a solid tumor antigen. The vector can comprise viral nucleic acid, such as lentiviral or AAV nucleic acid for delivering the nucleic acid encoding the CAR molecules. Examples of WBC antigens and solid tumor antigens are described herein. In embodiments, the WBC antigen is CD19. In embodiments the solid tumor antigen includes tumor associated MUC1 (tMUC1), PRLR, CLCA1, MUC12, GUCY2C, GPR35, CR1L, MUC 17, TMPRSS11B, MUC21, TMPRSS11E, CD207, SLC30A8, CFC1, SLC12A3, SSTR1, GPR27, FZD10, TSHR, SIGLEC15, SLC6A3, KISS1R, CLDN18.2, QRFPR, GPR119, CLDN6, UPK2, ADAM12, SLC45A3, ACPP, MUC21, MUC16, MS4A12, ALPP, CEA, EphA2, FAP, GPC3, IL13-Ra2, Mesothelin, PSMA, ROR1, VEGFR-II, GD2, FR-α, ErbB2, EpCAM, EGFRvIII, B7-H3, or EGFR.

In embodiments, one or more nucleic acids of the fusogen can further comprise one or more vectors including the nucleic acid encoding IL-12, IL-6, and/or IFNγ. The fusosomes described can be used with the system and device described herein for delivering one or more nucleic acids of interest to a subject in need thereof, for example, for immune therapy or causing a T cell response. The fusosome comprising the fusogen can be mixed with the blood cells obtained from the subject for introducing the fusogen into the blood cells so that the transduced blood cells can be reinfused into the subject in need thereof.

In embodiments, the one or more vectors can be modified for decreased cytotoxicity and phagocytosis mediated by PBMC cells, evading complement, reducing immunogenicity, and thus improving transduction efficiency. For example, modifications associated with HLA-G or HLA-E to have decreased cytotoxicity mediated by PBMC cell lysis, modifications associated with CD47 can evade macrophage phagocytosis, and/or modifications associated with immunogenic protein knockdown to reduce immunogenicity.

TABLE 3
Sequence IDs and corresponding identifiers

| Identifiers | SEQ ID NO: |
|---|---|
| SP | 1 |
| Hinge & transmembrane domain | 2 |
| 41-BB Co-stimulatory domain | 3 |
| CD3-zeta | 4 |
| CD19 CAR | 5 |
| TSHR CAR | 6 |
| GCC CAR 1 | 7 |
| Linker | 8 |
| GCC CAR 2 (CD8 signal peptide-VL-linker-VH- | 9 |
| CD8 hinge & transmembrane- CD137- CD3z) | |

The related sequences are provided in Innovative Cellular Therapeutics' PCT Patent Applications Nos: PCT/CN2016/075061, PCT/CN2018/08891, and PCT/US19/13068, which are incorporated herein by reference in their entirety.

The present disclosure is further described by reference to the following exemplary embodiments and examples. These exemplary embodiments and examples are provided for purposes of illustration only and are not intended to be limiting unless otherwise specified. Thus, the present disclosure should in no way be construed as being limited to the following exemplary embodiments and examples but rather should be construed to encompass any and all variations which become evident as a result of the teaching provided herein.

### EXEMPLARY EMBODIMENTS

The following are exemplary embodiments:
1. A device or system for immune therapy or causing a T cell response, the device comprising: a sample processing module configured to obtain a blood sample from a subject, the blood sample comprising T cells; a cell incubation module configured to: receive the blood sample, mix the blood sample with one or more vectors and an agent that activates T cells to introduce the one or more vectors into cells of the blood sample, and wash the blood sample to remove the one or more nontransduced vectors; and a cell infusion module configured to infuse at least a portion of the blood sample to the subject, wherein the sample processing module is coupled to the cell incubation module that is coupled to the cell infusion module such that the portion of the blood sample flow through the device starting from the subject and ending back to the subject.
2. The device or system of embodiment 1, wherein the blood sample comprises substantial whole blood, and the portion of the blood sample comprises at least two of CD3+ cells, NK cells, myeloid cells, and Neutrophil.
3. The device or system of embodiment 2, wherein the cell incubation module is further configured to remove one or more blood cells from the blood sample, and the one or more blood cells comprise B cells.
4. The device or system of embodiment 2, wherein the cell incubation module is further configured to remove one or more blood cells from the blood sample using a bead conjugated with an antibody against a B cell marker.
5. The device or system of embodiment 1, wherein the blood sample comprises PBMCs.
6. The device or system of embodiment 1, wherein the agent comprises a T cell activator.
7. The device or system of embodiment 1, wherein the agent comprises a bead conjugated with anti-CD3 and a bead conjugated with anti-CD28.
8. The device or system of embodiment 1, wherein the agent comprises Transact™.
9. The device or system of embodiment 1, wherein the one or more vectors are in a form of a viral particle that comprises a membrane-bound T cell activation element on a surface of the viral particle.
10. The device or system of embodiment 9, wherein the membrane-bound T cell activation element comprises or is anti-CD3.
11. The device or system of embodiment 9, wherein the viral particle further comprises a membrane-bound polypeptide capable of binding to CD28.
12. The device or system of any one of embodiments 1-11, wherein the device comprises a material chosen from at least one of plastics, polystyrol, polystyrene, polyvinylchloride, polycarbonate, glass, polyacrylate, polyacrylamide, polymethylmethacrylate (PMMA), polyethylene terephthalate (PET), polytetrafluoroethylene (PTFE), thermoplastic polyurethane (TPU), silicone, polyethylene (PE), collagen, chitin, alginate, hyaluronic acid derivatives, polylactide (PLA), polyglycolide (PGA) and their copolymers, polystyrol, polystyrene, polycarbonate, polyacrylate, ceramics, glass materials, like hydroxyapatite (HA), and calcium phosphate.
13. The device or system of any one of embodiments 1-12, wherein the device further comprises one or more sensors configured to detect progress of separation of the blood sample from the subject, in particular by detecting formation of layers of the blood sample, a change of pH value of the blood sample, and/or a change in temperature of the blood sample.
14. The device or system of any one of embodiments 1-12, wherein the cell incubation module comprises a rotating container configured to culture cells, and/or grow cells, and wherein the rotating container is disposable and/or has been sterilized.
15. The device or system of any one of embodiments 1-14, wherein a vein-to-vein time is between 30 minutes and 1 hour (hr), 1 hr and 72 hours (hrs), 1 hr and 12 hrs, 1 hr, and 24 hrs, 1 hr, and 48 hrs, 12 hrs and 24 hrs, 12 hr and 48 hrs, or 48 and 72 hrs (including numbers between).
16. The device or system of any one of embodiments 1-14, wherein a vein-to-vein time is between 30 minutes and 12 hrs or 12 hrs and 24 hrs. 17. The device of any one of embodiments 1-16, wherein the one or more vectors comprise a polynucleotide encoding a CAR targeting a WBC antigen and a polynucleotide encoding a CAR targeting a solid tumor antigen.
18. The device or system of any one of embodiments 1-17, wherein the one or more vectors further comprise a polynucleotide encoding IL-12, a polynucleotide encoding IL-6, and/or a polynucleotide encoding IFNγ.
19. The device or system of any one of embodiments 1-17, wherein the one or more vectors comprise a polynucleotide encoding a CAR targeting a solid tumor antigen, and the solid tumor antigen includes tumor associated MUC1 (tMUC1), PRLR, CLCA1, MUC12, GUCY2C, GPR35, CR1L, MUC 17, TMPRSS11B, MUC21, TMPRSS11E, CD207, SLC30A8, CFC1, SLC12A3, SSTR1, GPR27, FZD10, TSHR, SIGLEC15, SLC6A3, KISS1R, CLDN18.2, QRFPR, GPR119, CLDN6, UPK2, ADAM12, SLC45A3, ACPP, MUC21, MUC16, MS4A12, ALPP, CEA, EphA2, FAP, GPC3, IL13-Ra2, Mesothelin, PSMA, ROR1, VEGFR-II, GD2, FR-α, ErbB2, EpCAM, EGFRvIII, B7-H3, or EGFR.
20. The device or system of embodiment 19, wherein the CAR comprises an antigen binding domain, a transmembrane domain, a co-stimulatory domain, and a CD3 zeta domain.
21. The device or system of embodiment 20, wherein the co-stimulatory domain comprises an intracellular domain of CD27, CD28, 4-1BB, OX40, CD30, CD40, PD-1, ICOS, lymphocyte function-associated antigen-1 (LFA-1), CD2, CD7, LIGHT, NKG2C, B7-H3, a ligand that binds CD83, or a combination thereof.
22. A method of CAR T therapy, the method comprising: obtaining a blood sample from a subject, the blood sample comprising substantial whole blood; removing one or more cells from the blood sample to obtain a group of cells comprising at least CD3+ cells, NK cells, myeloid cells, and Neutrophils, the one or more cells comprising B cells; mixing the group of cells with one or more vectors and an agent that activates T cells to introduce the one or more vectors into the group of cells; and infuse at least a portion of the group of cells to the subject.
23. A method of CAR T therapy, the method comprising: obtaining a blood sample from a subject, the blood sample comprising substantial whole blood; mixing the blood sample with one or more vectors and an agent that activates T cells to introduce the one or more vectors into cells of the blood sample; removing one or more cells from the blood sample to obtain a group of cells comprising at least CD3+ cells, NK cells, myeloid cells, and Neutrophils, the one or more cells comprising B cells; and infuse at least a portion of the blood sample to the subject.
24. The method of embodiment 22 or 23, wherein a vein-to-vein time is between 30 minutes and 1 hour (hr), 1 hr and 72 hours (hrs), 1 hr and 12 hrs, 1 hr, and 24 hrs, 1 hr, and 48 hrs, 12 hrs and 24 hrs, 12 hr and 48 hrs, or 48 and 72 hrs (including numbers between).
25. The method of embodiment 22 or 23, wherein a vein-to-vein time is between 30 minutes and 12 hrs or 12 hrs and 24 hrs. 26. The method of embodiment 22 or 23, wherein the one or more vectors comprise a polynucleotide encoding a CAR targeting a WBC antigen and a polynucleotide encoding a CAR targeting a solid tumor antigen.
27. The method of embodiment 22 or 23, wherein the one or more vectors further comprise a polynucleotide encoding IL-12, a polynucleotide encoding IL-6, and/or a polynucleotide encoding IFNγ.
28. A method of enhancing treatment of cancer, the method comprising: obtaining a whole blood sample; mixing the whole blood sample with a medium comprising a viral vector nucleic acid sequences encoding a CAR and IL-12 to obtain modified blood cells; and administering the modified blood cells to a subject having a form of cancer, wherein the treatment on the subject is enhanced as compared with a subject that is administered with nucleic acid sequences encoding the CAR without IL-12.
29. The method of embodiment 28, wherein the medium comprises protamine sulfate (PS) and LentiBOOST™, the treatment on the subject is enhanced as compared with a subject that is administered with the pharmaceutical composition without including the PS and LentiBOOST™
30. A method of enhancing treatment of cancer, the method comprising: obtaining a whole blood sample; mixing the whole blood sample with a medium comprising a viral vector nucleic acid sequences encoding a CAR to obtain modified blood cells; and administering the modified blood cells to a subject having a form of cancer, wherein the medium comprises protamine sulfate (PS) and LentiBOOST™, the treatment on the subject is enhanced as compared with a subject that is administered with the pharmaceutical composition without including the PS and LentiBOOST™
31. The method of embodiment 28, wherein the nucleic acid sequences further encode IL-12, and the treatment on the subject is enhanced as compared with a subject that is administered with nucleic acid sequences encoding the CAR without IL-12.
32. A method of enhancing treatment of cancer, the method comprising: administering a pharmaceutical composition comprising a viral vector comprising nucleic acid sequences encoding a CAR and IL-12 to a subject having a form of cancer, wherein the treatment on the subject is enhanced as compared with a subject that is administered with nucleic acid sequences encoding the CAR without IL-12.
33. The method of embodiment 32, wherein the pharmaceutical composition comprises protamine sulfate (PS) and LentiBOOST™, the treatment on the subject is enhanced as compared with a subject that is administered with the pharmaceutical composition without including the PS and LentiBOOST™.
34. A method of enhancing treatment of cancer, the method comprising: administering a pharmaceutical composition comprising a viral vector comprising nucleic acid sequences encoding a CAR to a subject having a form of cancer, wherein the pharmaceutical composition comprises protamine sulfate (PS) and LentiBOOST™, the treatment on the subject is enhanced as compared with a subject that is administered with the pharmaceutical composition without including the PS and LentiBOOST™.
35. The method of embodiment 32, wherein the nucleic acid sequences further encode IL-12, and the treatment on the subject is enhanced as compared with a subject that is administered with nucleic acid sequences encoding the CAR without IL-12.
36. The method of any one of embodiments 32-35, wherein cells, organs, or tissue of the peripheral blood system express the antigen binding molecule of the subject.
37. The method of any one of embodiments 32-36, further comprising monitoring T cell response derived from the expression of the CAR in the subject.
38. The method of any one of embodiments 28-37, wherein the enhanced treatment comprises enhanced anti-tumor activities and/or a survival time of the subject.
39. The method of any one of embodiments 28-38, wherein the viral vector comprises a lentivirus.
40. The method of any one of embodiments 32-39, wherein the viral vector is an rAAV particle comprising an AAV capsid protein and a vector comprising the nucleic acid encoding antigen binding molecule inserted between a pair of AAV inverted terminal repeats (ITRs) in a manner effective to infect the cells, organ or tissue of the peripheral system in the subject such that the cells, organ or tissue express antigen binding molecule.
41. The method of any one of embodiments 32-40, wherein the nucleic acid encoding IL-12 has reduced cytosine-guanine dinucleotide (CpG) compared to a non-CpG reduced nucleic acid encoding the therapeutic protein.
42. The method of any one of embodiments 32-40, wherein the nucleic acid encoding IL-12 has reduced cytosine-guanine dinucleotide (CpG) compared to a wild-type nucleic acid encoding the therapeutic protein.
43. The method of any one of embodiments 32-42, wherein the vector further comprises one or more of an intron, an expression control element, a filler polynucleotide sequence and/or poly A signal, or a combination thereof.
44. The method of any one of embodiments 40-43, wherein the pair of ITRs are derived from or comprises a sequence of AAV1, AAV2, AAV3, AAV4, AAV5, AAV6, AAV7, AAV8, AAV9, AAV10, AAV11, AAV 12, LK01, LK02, LK03, AAV 4-1, AAV-218 ITRs, or a mixture thereof.
45. The method of any one of embodiments 40-44, wherein the AAV capsid protein is derived from or comprise a sequence of AAV1, AAV2, AAV3, AAV4, AAV5, AAV6, AAV7, AAV8, AAV9, AAV10, AAV11, AAV 12, LK01, LK02, LK03, AAV 4-1, AAV-218 ITRs, or a mixture thereof.
46. The method of any one of embodiments 40-45, wherein the rAAV particles are administered at a dose in a range from about 1×108-1×1010, 1×1010-1×1011, 1×1011-1×1012, 1×1012-1×1013, or 1×1013-1×1014 vector genomes per kilogram (vg/kg) of the subject.
47. The method of any one of embodiments 40-46, wherein the rAAV particles are administered at the dose of embodiment 47 of less than 1×10 vector genomes per kilogram (vg/kg) of the subject.
48. The method of any of embodiments 40-46, wherein the rAAV particles are administered at a dose of about 5×1011 vector genomes per kilogram (vg/kg) of the subject.
49. The method of any one of embodiments 40-48, wherein the rAAV particles administered vector genomes (vg) per kilogram (vg/kg) of the weight of the subject, or between about 1×1010 to 1×1011 vg/kg of the weight of the subject, or between about 1×1011 to 1×1012 vg/kg (e.g., about 1×1011 to 2×1011 vg/kg or about 2×1011 to 3×1011 vg/kg or about 3×1011 to 4×1011 vg/kg or about 4×1011 to 5×1011 vg/kg or about 5×1011 to 6×1011 vg/kg or about 6×1011 to 7×1011 vg/kg or about 7×1011 to 8×1011 vg/kg or about 8×1011 to 9×1011 vg/kg or about 9×1011 to 1×1012 vg/kg) of the weight of the subject, or 12-13 between about 1×10 to 1×10 vg/kg of the weight of the subject, to achieve a desired therapeutic effect.
50. The method of any one of embodiments 32-49, wherein the subject does not develop a substantial immune response against the rAAV particle.
51. The method of any one of embodiments 32-49, wherein the subject does not develop a substantial humoral immune response against the rAAV particle.
52. The method of any one of embodiments 32-49, wherein the subject does not develop a substantial immune response against the rAAV particle for at least 1, 2, 3, 4, 5, 6, 7, 8, 9, 10, 11, 12, 13 or 14 continuous days, weeks or months.
53. The method of any one of embodiments 32-49, wherein the subject does not develop a detectable immune response against the rAAV particle.
54. The method of any one of embodiments 32-49, wherein the subject does not produce an immune response against the therapeutic protein and/or the rAAV particle sufficient to block the therapeutic effect in the subject for at least 1, 2, 3, 4, 5, 6, 7, 8, 9, 10, 11, 12, 13 or 14 continuous days, weeks or months.
55. The method of any of embodiments 32-54, wherein the administering comprises infusion or injection into the systemic circulation of the subject.
56. The method of any one of embodiments 32-54, wherein the administering comprises intravenous or intra-arterial infusion or injection into the systemic circulation of the subject.
57. The method of any of embodiments 28-56 wherein the CAR comprises an extracellular domain, a transmembrane domain, and an intracellular domain, the extracellular domain binds an antigen, the intracellular domain comprises a co-stimulatory signaling region that comprises an intracellular domain of a co-stimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, and any combination thereof, and the antigen is Epidermal growth factor receptor (EGFR), Variant III of the epidermal growth factor receptor (EGFRvIII), Human epidermal growth factor receptor 2 (HER2), Mesothelin (MSLN), Prostate-specific membrane antigen (PSMA), Carcinoembryonic antigen (CEA), Disialoganglioside 2 (GD2), Interleukin-13Ra2 (IL13Rα2), Glypican-3 (GPC3), Carbonic anhydrase IX (CAIX), L1 cell adhesion molecule (L1-CAM), Cancer antigen 125 (CA125), Cluster of differentiation 133 (CD133), Fibroblast activation protein (FAP), Cancer/testis antigen 1B (CTAG1B), Mucin 1 (MUC1), Folate receptor-α (FR-α), CD19, FZD10, TSHR, PRLR, Muc 17, GUCY2C, CD207, CD3, CD5, B-Cell Maturation Antigen (BCMA), or CD4.
58. The method of any of embodiments 28-57, wherein expression of IL-12 is regulated by NFAT promoter.
59. The method of any of embodiments 28-58, wherein the pharmaceutical composition or the medium further comprises CD28 and CD3 agonists.
60. The method of any of embodiments 28-58, wherein the pharmaceutical composition or the medium further comprises CD28 and CD3 antibodies.
61. The method of any of embodiments 28-58, wherein the pharmaceutical composition or the medium further comprises TransAct™.
62. A pharmaceutical composition for use in a method of enhancing treatment of cancer, the method comprising: administering the pharmaceutical composition comprising a viral vector comprising nucleic acid sequences encoding a CAR and IL-12 to a subject having a form of cancer, wherein the treatment on the subject is enhanced as compared with a subject that is administered with nucleic acid sequences encoding the CAR without IL-12.
63. The pharmaceutical composition for use of embodiment 62, wherein the pharmaceutical composition comprises protamine sulfate (PS) and LentiBOOST™, the treatment on the subject is enhanced as compared with a subject that is administered with the pharmaceutical composition without including the PS and LentiBOOST™
64. A pharmaceutical composition for use in a method of enhancing treatment of cancer, the method comprising: administering a pharmaceutical composition comprising a viral vector comprising nucleic acid sequences encoding a CAR to a subject having a form of cancer, wherein the pharmaceutical composition comprises protamine sulfate (PS) and LentiBOOST™ the treatment on the subject is enhanced as compared with a subject that is administered with the pharmaceutical composition without including the PS and LentiBOOST™.
65. The pharmaceutical composition for use of embodiment 64, wherein the nucleic acid sequences further encode IL-12, and the treatment on the subject is enhanced as compared with a subject that is administered with nucleic acid sequences encoding the CAR without IL-12.
66. Modified blood cells for use in a method of enhancing treatment of cancer, the method comprising: obtaining a whole blood sample; mixing the whole blood sample with a medium comprising a viral vector nucleic acid sequences encoding a CAR and IL-12 to obtain modified blood cells; and administering the modified blood cells to a subject having a form of cancer, wherein the treatment on the subject is enhanced as compared with a subject that is administered with nucleic acid sequences encoding the CAR without IL-12.
67. The modified blood cells for use of embodiment 66, wherein the medium comprises protamine sulfate (PS) and LentiBOOST™, the treatment on the subject is enhanced as compared with a subject that is administered with the pharmaceutical composition without including the PS and LentiBOOST™
68. Modified blood cells for use in a method of enhancing treatment of cancer, the method comprising: obtaining a whole blood sample; mixing the whole blood sample with a medium comprising a viral vector nucleic acid sequences encoding a CAR to obtain modified blood cells; and administering the modified blood cells to a subject having a form of cancer, wherein the medium comprises protamine sulfate (PS) and LentiBOOST™, the treatment on the subject is enhanced as compared with a subject that is administered with the pharmaceutical composition without including the PS and LentiBOOST™.
69. The modified blood cells for use of embodiment 28, wherein the nucleic acid sequences further encode IL-12, and the treatment on the subject is enhanced as compared with a subject that is administered with nucleic acid sequences encoding the CAR without IL-12.
70. The method of any suitable embodiment of embodiments 1-69, wherein the modified blood cells do not include B cells.
71. The method of any suitable embodiment of embodiments 1-70, wherein the modified blood cells comprise T cells, NK cells, myeloid cells, and Neutrophil.
72. The method of any suitable embodiment of embodiments 1-70, wherein the modified blood cells comprise T cells, NK cells, and Neutrophil.
73. A method of enhancing in vivo transduction of T cells or enhancing a gene therapy, the method comprising: administering to a subject having a form of cancer with an effective of a pharmaceutical composition comprising a viral vector comprising a nucleic acid encoding a CAR or TCR and one or more compounds, the one or more compounds comprising a T cell activator and/or a transduction adjuvant; and allowing the viral vector to be introduced into the T cells of the subject, wherein a transduction rate of the nucleic acid is greater than T cells of a subject that is administered with an effective of a pharmaceutical composition comprising the viral vector without the one or more compounds.
74. A method of performing or enhancing a gene therapy, the method comprising: administering to a subject having a form of cancer with an effective of a pharmaceutical composition comprising a viral vector comprising a nucleic acid encoding a CAR or TCR and one or more compounds, the one or more compounds comprising a T cell activator and a transduction adjuvant; and allowing the viral vector to be introduced into the T cells of the subject.
75. The method of embodiment 73 or 74, wherein the T cell activator comprises anti-CD3 and/or anti-CD28 antibodies.
76. The method of embodiment 73 or 74, wherein the one or more compounds comprise antibodies or agonists binding CD3 and/or CD28 and PS.
77. The method of any one of embodiments 73-76, wherein the viral vector comprising a nucleic acid encoding IL-12, wherein anti-tumor activities of the subject is greater than a subject that is administered with a viral vector comprising the nucleic acid encoding the CAR and not comprising the nucleic acid encoding IL-12.
78. The method of any one of embodiments 73-77, wherein the CAR binds CD19.
79. The method of any one of embodiments 73-78, wherein the form of cancer comprises lymphoma.
80. The method of any one of embodiments 73-79, wherein the viral vector comprises a nucleic acid encoding a dominant negative form of PD-1.
81. The method of any suitable preceding embodiment, wherein the viral vector(s) include a nucleic acid encoding a CAR targeting a WBC antigen (e.g., CD19 and BCMA) and a nucleic acid encoding a CAR targeting a solid tumor antigen (e.g., GCC and TSHR).
81. The method of any suitable preceding embodiment, wherein the viral vector(s) can include (1) a nucleic acid encoding a CAR targeting a WBC antigen (e.g., CD19 and BCMA) and IL-6, a nucleic acid encoding the CAR targeting the WBC antigen and IL-12, or a nucleic acid encoding the CAR targeting the WBC antigen and IFN-γ, or a combination thereof, and (2) a nucleic acid encoding a CAR targeting a solid tumor antigen (e.g., GCC and TSHR).
82. A method of enhancing T cell response in a subject or treating a subject having cancer, the method comprising: administering an effective amount of a composition comprising modified cells to the subject having a form of cancer associated with or expressing an antigen. (e.g., a solid tumor antigen); and administering an effective amount of: a composition comprising one or more nucleic acids encoding the antigen or a variant thereof, a composition comprising additional modified cells and/or microorganisms (e.g., cold viruses) comprising: the one or more nucleic acids or the antigen or a variant thereof.
83. The method of embodiment 82, wherein the modified cells comprise modified T cells, modified NK cells, modified macrophages, or modified dendritic cells.
84. The method of embodiment 82, wherein the modified cells comprise at least two different modified cells: a first modified cell comprising an antigen binding domain for expanding and/or maintaining the modified cells; and a second modified cell comprising an antigen binding domain for killing a target cell, such as a tumor cell (e.g., the solid tumor antigen).
85. The method of embodiment 84, wherein the modified cells are modified T cells.
86. The method of embodiment 84, wherein the at least two different modified cells include two different modified T cells, two different modified immune cells, or a combination thereof.
87. The method of embodiment 84, wherein the modified immune cells include modified T cells, DC cells, and/or macrophages.
88. The method of embodiment 84, wherein the antigen binding domain for expanding/or and maintaining the modified cells bind the surface antigen of a WBC, and the antigen binding domain for killing a target cell binds a tumor antigen.
89. The method of embodiment 88, wherein the WBC is a B cell.
90. The method of embodiment 88, wherein the cell surface antigen of the WBC is CD19, CD22, CD20, BCMA, CD5, CD7, CD2, CD16, CD56, CD30, CD14, CD68, CD11b, CD18, CD169, CD1c, CD33, CD38, CD138, or CD13.
91. The method of embodiment 88, wherein the cell surface antigen of the WBC is CD19, CD20, CD22, or BCMA.
92. The method of any of embodiments 82-91, wherein the solid tumor antigen is tMUC1, PRLR, CLCA1, MUC12, GUCY2C, GPR35, CR1L, MUC 17, TMPRSS11B, MUC21, TMPRSS11E, CD207, SLC30A8, CFC1, SLC12A3, SSTR1, GPR27, FZD10, TSHR, SIGLEC15, SLC6A3, KISS1R, QRFPR, GPR119, CLDN6, UPK2, ADAM12, SLC45A3, ACPP, MUC21, MUC16, MS4A12, ALPP, CEA, EphA2, FAP, GPC3, IL13-Ra2, Mesothelin, PSMA, ROR1, VEGFR-II, GD2, FR-α, ErbB2, EpCAM, EGFRvIII, B7-H3, EGFR, or one of those listed in Table 1.
93. The method of any of embodiments 82-91, wherein the solid tumor antigen is tMUC1, TSHR, GUCY2C, ACPP, CLDN18.2, PSMA, MAGE A4, MSLN, CD205, ADAM12, GPC-3, or UPK2.
94. The method of embodiment 82, wherein the modified cells comprise an exogenous polynucleotide encoding a therapeutic agent.
95. The method of embodiment 94, wherein the exogenous polynucleotide comprises a promoter comprising a binding site for a transcription modulator that modulates the expression and/or secretion of IL-6, IFNγ, or a combination thereof, in the modified cell.
96. The method of embodiment 94, wherein the exogenous polynucleotide comprises a promoter comprising a binding site for a transcription modulator that modulates the expression and/or secretion of IL-6 and IFNγ.
97. The method of embodiment 95, wherein the transcription modulator comprises Hif1a, NFAT, FOXP3, or NFkB.
98. The method of any of embodiments 94-97, wherein the therapeutic agent comprises at least one of IL-1P, IL-2, IL-4, IL-5, IL-6, IL-7, IL-8, IL-10, IL-12, IL-13, IL-15, IL-17, IL-1Ra, IL-2R, IFN-γ, IFN-γ, MIP-In, MIP-IP, MCP-1, TNFα, GM-CSF, GCSF, CXCL9, CXCL10, CXCR factors, VEGF, RANTES, EOTAXIN, EGF, HGF, FGF-P, CD40, CD40L, and ferritin.
99. The method of any of embodiments 94-97, wherein the therapeutic agent comprises a co-stimulatory structure, activating antibody, or ligand agonist, such as CD205, CD40L, CD28L, CD137L, or a dominant negative form of an immune checkpoint molecule. In embodiments, the therapeutic agent is expressed by a target cell of the one or more nucleic acids (e.g., APC such as B cell or DC, or T cell).
100. The method of any of embodiments 82-99, wherein the modified cell comprises a modified immune checkpoint molecule (e.g., PD-1).
101. The method of embodiment 100 wherein the immune checkpoint molecule is selected from the group consisting of PD-1, cytotoxic T lymphocyte antigen-4 (CTLA-4), B- and T-lymphocyte attenuator (BTLA), T-cell immunoglobulin mucin-3 (TIM-3), lymphocyte-activation protein 3 (LAG-3), T-cell immunoreceptor with Ig and ITIM domains (TIGIT), leukocyte-associated immunoglobulin-like receptor 1 (LAIRI), natural killer cell receptor 2B4 (2B4), and CD 160.
102. The method of embodiment 100 wherein a modified PD-1 is a dominant negative form of PD-1.
103. The method of embodiment 100 wherein modified PD-1 comprises an extracellular domain of PD-1 and a cytoplasmic domain of the PD-1 polypeptide is truncated, or the modified cell has a partial or complete deletion of the PD-1 gene and a reduced amount of PD-1 as compared to the corresponding wild-type cell, or a non-functional PD-1 gene.
104. The method of embodiment 100 wherein the modified PD-1 comprises a mutation of Tyrosine residue 223 and/or a mutation of Tyrosine residue 248.
105. The method of any of embodiments 82-104, wherein the modified cells comprise a binding molecule binding the antigen.
106. The method of embodiment 105, wherein the binding molecule is a chimeric antigen receptor.
107. The method of embodiment 106, wherein the CAR comprises an extracellular domain, a transmembrane domain, and an intracellular domain, and the extracellular domain binds a tumor antigen.
108. The method of embodiment 106, wherein the intracellular domain comprising a co-stimulatory domain that comprises an intracellular domain of a co-stimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, or a combination thereof, and/or wherein the intracellular domain comprises a CD3 zeta signaling domain.
109. The method of embodiment 105, wherein the binding molecule is a TCR.
110. The method of embodiment 105, wherein the modified cells comprise a modified T Cell Receptor (TCR).
111. The method of embodiment 109 or 110, wherein the TCR is derived from spontaneously occurring tumor-specific T cells in patients.
112. The method of embodiment 109 or 110, wherein the TCR binds a tumor antigen.
113. The method of embodiment 109 or 110, wherein the tumor antigen comprises CEA, gp100, MART-1, p53, MAGE-A3, or NY-ESO-1.
114. The method of embodiment 109 or 110, wherein the TCR comprises TCRγ and TCRδ chains, TCRα and TCRβ chains, or a combination thereof.
115. The method of any of embodiments 82-114, wherein the modified cells are derived from TILs.
116. The method of embodiment 82, wherein the additional modified cells comprise PBMCs, blood cells (red blood cells), DCs, B cells, and/or T cells.
117. The method of embodiment 116, wherein the additional modified cells are formulated to a vaccine. More information about the formulation of vaccines can be found at B-cell epitope peptide cancer vaccines: a new paradigm for combination immunotherapies with novel checkpoint peptide vaccine, Pravin T P Kaumaya, Future Oncology 2020 16:23, 1767-1791, Companion vaccines for CAR T-cell therapy: applying basic immunology to enhance therapeutic efficacy; Adam E Snook, Future Medicinal Chemistry 2020 12:15, 1359-1362, which are incorporated by their entirety.
118. The method of embodiment 82, wherein the one or more nucleic acids further comprise a polynucleotide encoding the therapeutic agent described in preceding embodiments. More information about modified mRNA expressing therapeutic agent to leukocytes can be found at Veiga, N., Goldsmith, M., Granot, Y. et al. Cell specific delivery of modified mRNA expressing therapeutic proteins to leukocytes. Nat Commun 9, 4493 (2018), which is incorporated by their entirety.
119. The method of any of embodiments 82-118, wherein a SynNotch system is added to the one or more nucleic acids to allow DC cells to express antigen outside the cell and connect intracellularly with genes that activate cells or enhance cell killing, such as cytokines and co-stimulatory ligands, and when the extracellular antigen recognizes the tumor, it will cause the intracellular SynNotch signal to activate the cell and enhance the CAR-T killing function.
120. The method of any of embodiments 82-119, wherein the one or more nucleic acids are in vitro transcribed RNA disposed in liposomes.
121. The method of embodiment 120, wherein the liposomes comprise N-[1-(2,3-dioleyloxy) propyl]-N,N, N-trimethylammonium chloride (DOTMA), dioleoylphosphatidylethanolamine (DOPE), or DOTMA and cholesterol.
122. The method of any of embodiments 82-121, wherein the antigen comprises a non-essential tissue antigen. More information about the non-essential tissue antigen can found at a U.S. patent Ser. No. 10/793,638B.
123. The method of any of embodiments 82-121, wherein the antigen comprises a tumor-associated antigen (TAA). More information about the non-essential tissue antigen can found at a PCT Patent Publication NO: WO2020146743, which is incorporated by their entirety.
124. The method of any of embodiments 82-121, wherein the antigen comprises a neoantigen, a class of tumor-specific antigens, that differs from the traditional tumor-associated antigen (TAA). TAA is not unique to tumor tissue as it is also present in normal tissues; it is highly expressed in proliferating tumor cells expressing HER2, MART-1, MUC1, and MAGE (Li L, Goedegebuure S P, Gillanders W E. Preclinical and clinical development of neoantigen vaccines. Ann Oncol. 2017; 28: xii11-7), which is incorporated by their entirety.
125. The method of any of embodiments 82-124, wherein the antigen or the one or more nucleic acids are delivered into cells using the systems and methods described in the U.S. patent Ser. No. 10/526,573, which is incorporated by its entirety.
126. The method of any of embodiments 82-125, wherein the one or more nucleic acids are inserted into the genome of the microorganism (e.g., vaccinia viruses).
127. A polynucleotide(s) comprising the one or more nucleic acids of any of embodiments 82-126.
128. A cell comprising the polynucleotide(s) of embodiment 127.
129. A method of enhancing T cell response in a subject or treatment of a subject having cancer, the method comprising:
administering an effective amount of a composition comprising CAR T cells to the subject having a form of cancer associated with or expressing an antigen (e.g., a solid tumor antigen) that the CAR binds; and
administering one or more nucleic acids encoding the antigen or a variant thereof, a level of T cell response or anti-tumor activities caused by the CAR T cells enhanced as compared to a subject that is administered with the CAR T cells without the one or more nucleic acids.
130. A method of enhancing T cell response in a subject or treatment of a subject having cancer, the method comprising: administering an effective amount of a composition comprising first CAR T cells targeting a first antigen (e.g., a WBC antigen such as CD19 and BCMA) to the subject having a form of cancer associated with or expressing an antigen (e.g., a solid tumor antigen); administering an effective amount of a composition comprising second CAR T cells targeting the antigen to the subject; and in a predetermined time or in response to a predetermined condition, administering one or more nucleic acids encoding the antigen or a variant thereof, a level of T cell response or anti-tumor activities caused by the CAR T cells enhanced as compared to a subject that is administered with the CAR T cells without the one or more nucleic acids.
131. The method of embodiment 130, wherein the predetermined time comprises any one of 1-30 days after administration of the CAR T cells.
132. The method of embodiment 130 wherein the predetermined condition comprises a level of second CAR T cells or solid tumor antigen CAR copy numbers drop to a certain amount.
133. A method of enhancing T cell response in a subject or treatment of a subject having cancer, the method comprising: administering an effective amount of a composition comprising first CAR T cells targeting a first antigen (e.g., a WBC antigen such as CD19 and BCMA) to the subject having a form of cancer associated with or expressing an antigen (e.g., a solid tumor antigen); administering an effective amount of a composition comprising second CAR T cells targeting the antigen to the subject; and administering one or more nucleic acids encoding the antigen or a variant thereof, a level of T cell response or anti-tumor activities caused by the CAR T cells enhanced as compared to a subject that is administered with the CAR T cells without the one or more nucleic acids.
134. A kit comprising the vaccine, for example, described in embodiment 117, and CAR T cells of any of the preceding suitable embodiments.
135. The device, system, or method of any of the preceding suitable embodiments of embodiments 1-61, or 73-133, wherein the device, system, or method comprises using fusosome technology, for example, transducing the blood cells obtained from the subject with fusosome comprising fusogen including one or more nucleic acids encoding one or more suitable antigen binding molecules described herein.
136. The device, system, or method of embodiment 135, wherein the fusogen further comprises one or more nucleic acids encoding IL-12, IL-6, and/or IFNγ.
137. A fusosome comprising one or more fusogens, wherein the one or more fusogens includes one or more nucleic acids encoding one or more suitable antigen binding molecules described herein.
138. A pharmaceutical composition comprising the fusosome of embodiment 137.
139. Use of the fusosome or the pharmaceutical composition of embodiment 137 or 138, for (1) treating a subject in need thereof, (2) causing T cell response in a subject, (3) expanding T cells in a subject, (4) inhibiting growth of tumor cells in a subject, (5) genetically modifying lymphocytes, (6) treating tumors in a subject, and/or (7) enhancing T cell response, T cell expansion in vivo, inhibition growth of tumor cells, and/or treatment of the tumor.
140. Use of the device, system, method, pharmaceutical composition, polynucleotide (or nucleic acid), cells, modified cells, or fusosomes of any of the suitable preceding embodiments for (1) treating a subject in need thereof, (2) causing T cell response in a subject, (3) expanding T cells in a subject, (4) inhibiting growth of tumor cells in a subject, (5) genetically modifying lymphocytes, (6) treating tumors in a subject, and/or (7) enhancing T cell response, T cell expansion in vivo, inhibition growth of tumor cells, and/or treatment of the tumor.

### EXAMPLES

Modified Cells Derived from T Cells

Lentiviral vectors that encode individual CAR molecules were generated and transfected with T cells, which are described below. Techniques related to cell cultures, construction of cytotoxic T lymphocyte assay can be found in “Control of large, established tumor xenografts with genetically retargeted human T cells containing CD28 and CD137 domains,” PNAS, Mar. 3, 2009, vol. 106 no. 9, 3360-3365 and “Chimeric Receptors Containing CD137 Signal Transduction Domains Mediate Enhanced Survival of T Cells and Increased Antileukemic Efficacy In Vivo,” Molecular Therapy, August 2009, vol. 17 no. 8, 1453-1464, which are incorporated herein by reference in its entirety.

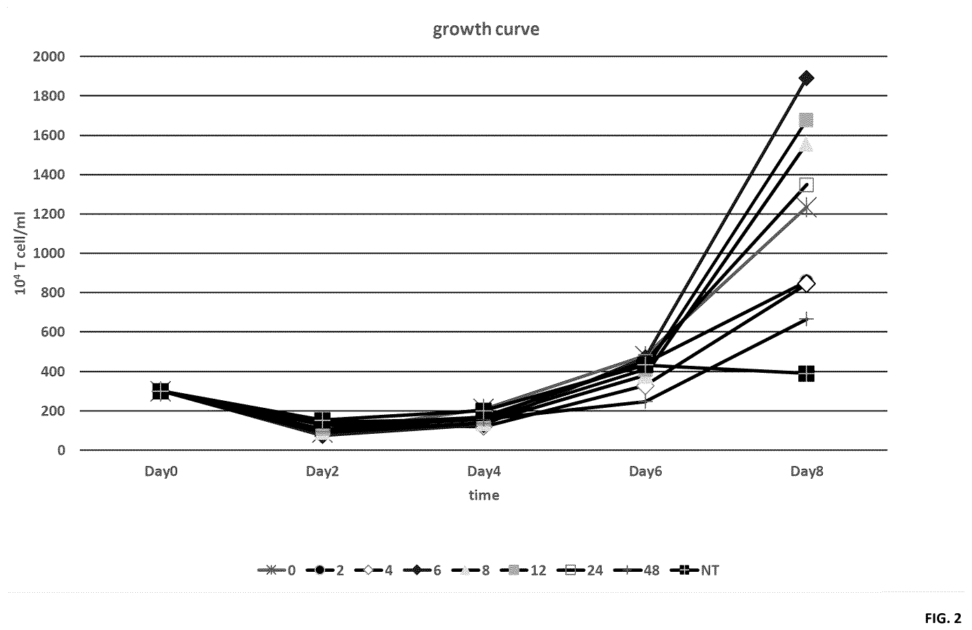

FIG. 2 shows growth curves of CD19 CAR T cells obtained in different manners. On day 0, T cells were obtained from volunteers. These T cells were activated using anti-CD3/CD28 conjugated beads and mixed with lentiviral vectors encoding SEQ ID NO: 5 for a different time period to obtain various CD19 CAR T cells, which were cultured and analyzed at different time points. As shown in FIG. 2, the proliferation of CD19 CAR T cells mixed with vectors for 6, 8, 12, and 24 hours appeared to be better than other time points. Further, CAR expression was evaluated, and results are provided in Table 4. The scFv of CD19 CAR is humanized scFv, and its sequence is disclosed Innovative Cellular Therapeutics' PCT Patent Applications Nos: PCT/CN2016/075061, PCT/CN2018/08891, and PCT/US19/13068, all of which are incorporated by reference in their entirety.

TABLE 4
CAR Expression
car expression

| | 0 h | 2 h | 4 h | 6 h | 8 h | 12 h | 24 h | 48 h | NT |
|---|---|---|---|---|---|---|---|---|---|
| day 4 | 14.34% | 17.55% | 13.61% | 12.31% | 10.83 | 15.58 | 21.89% | 29.65% | 3.18% |
| day 6 | 38.22% | 48.93% | 58.92% | 44.21% | 42.23% | 56.19% | 46.34% | 67.01% | 3.09% |
| day 8 | 39.24% | 51.04% | 56.89% | 40.04% | 40.16% | 50.97% | 47.99% | 61.18% | 5.87% |

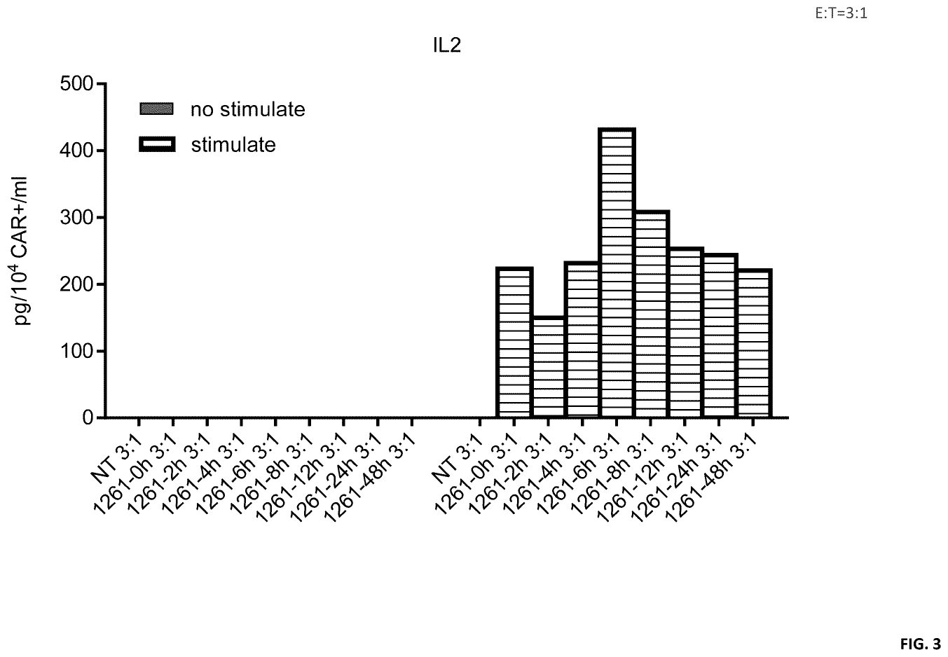

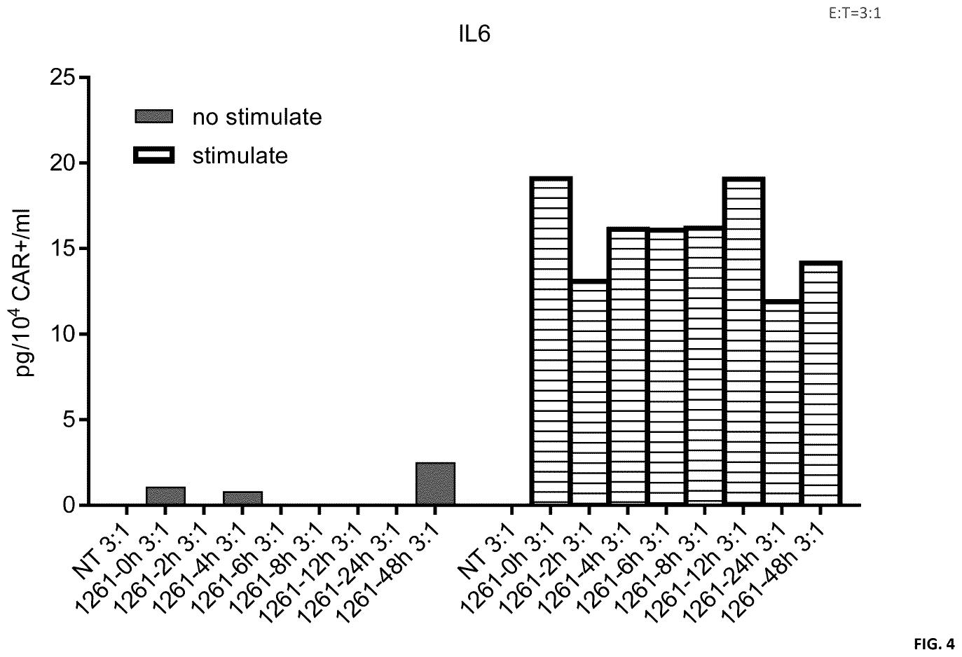

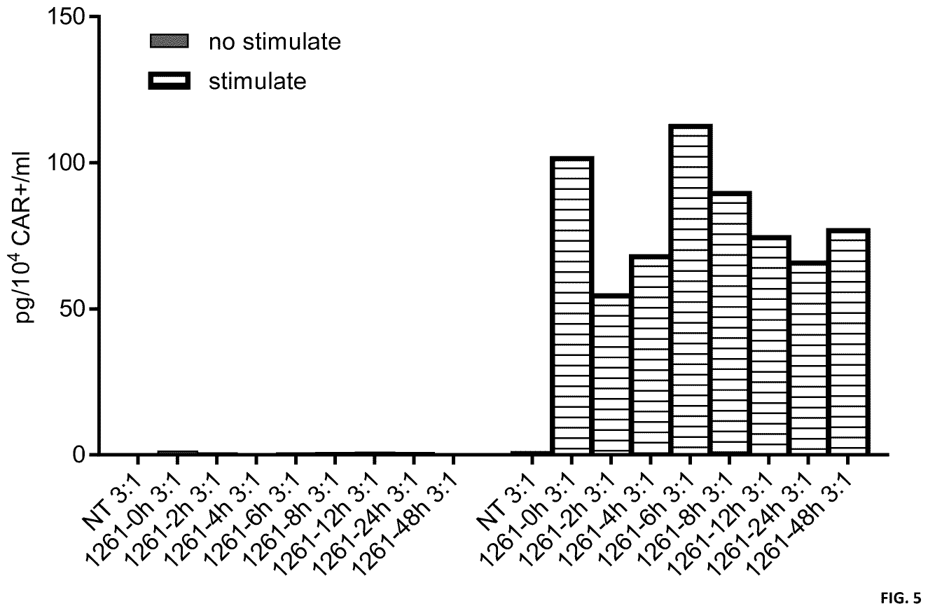

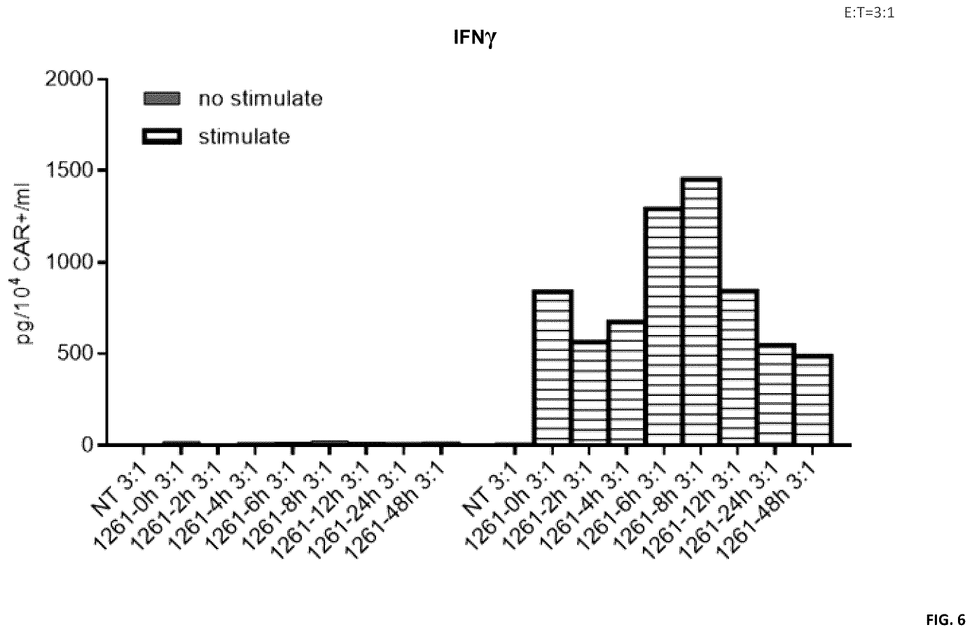

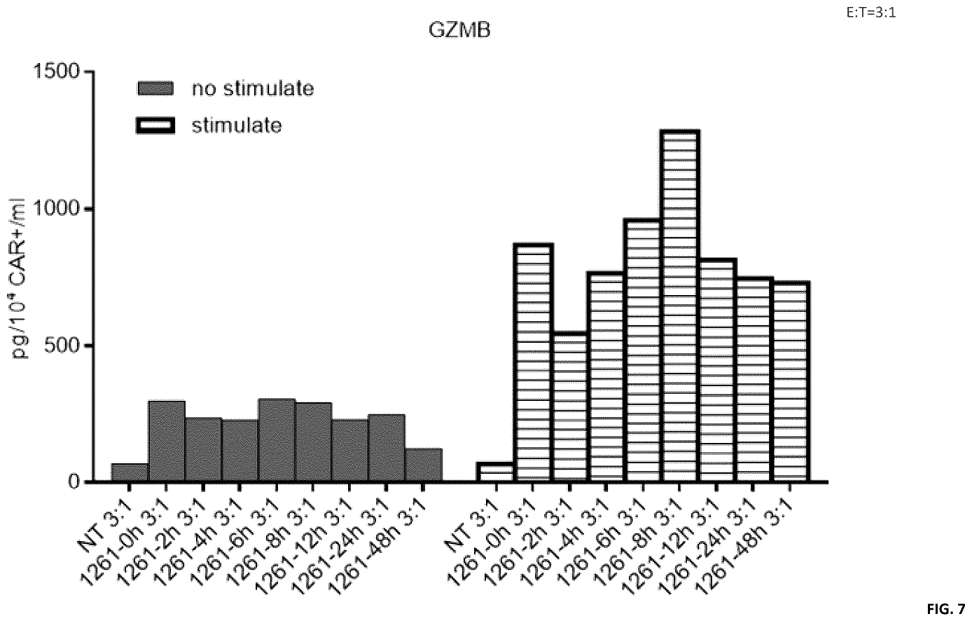

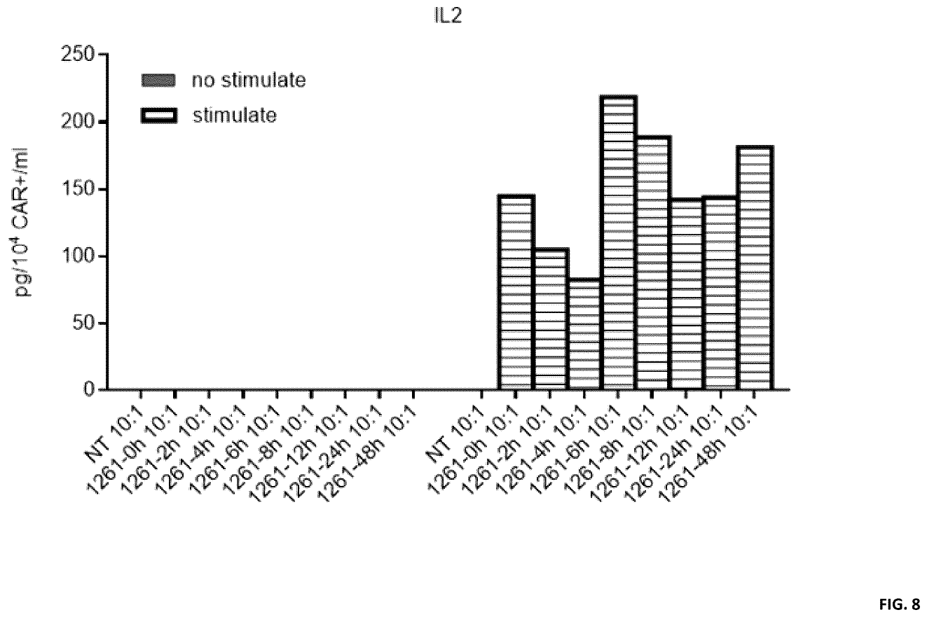

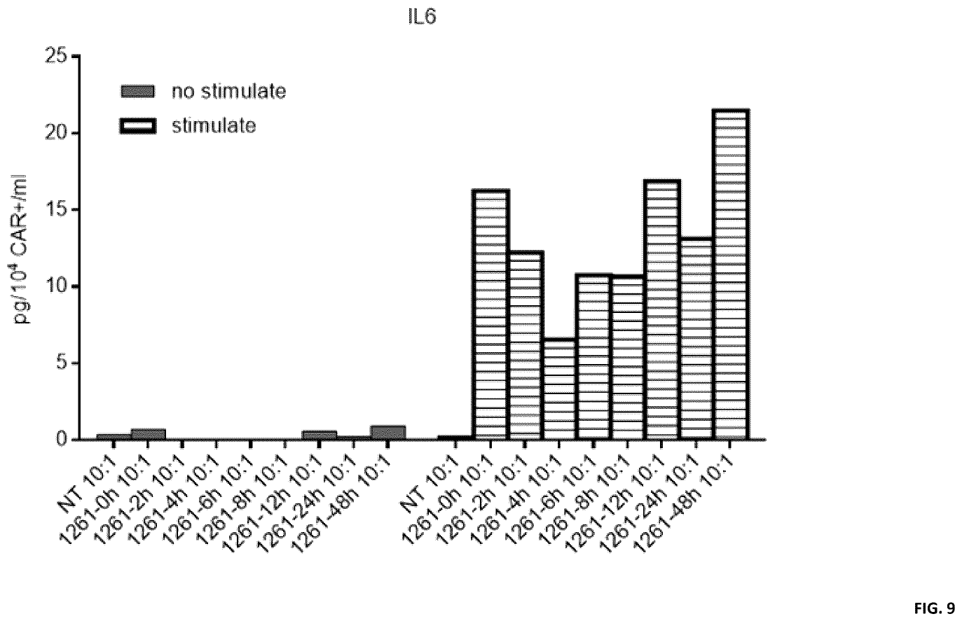

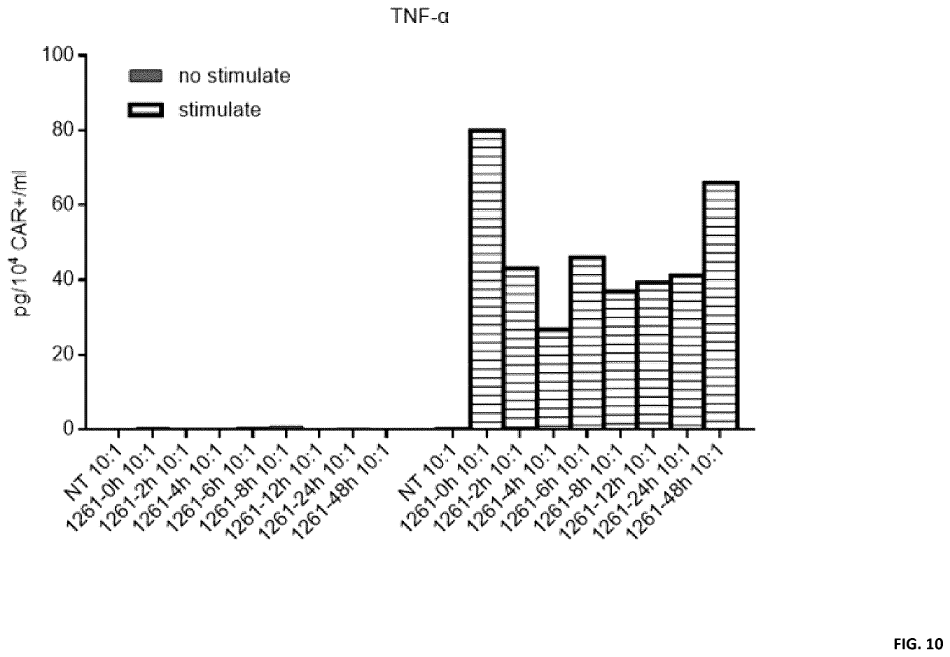

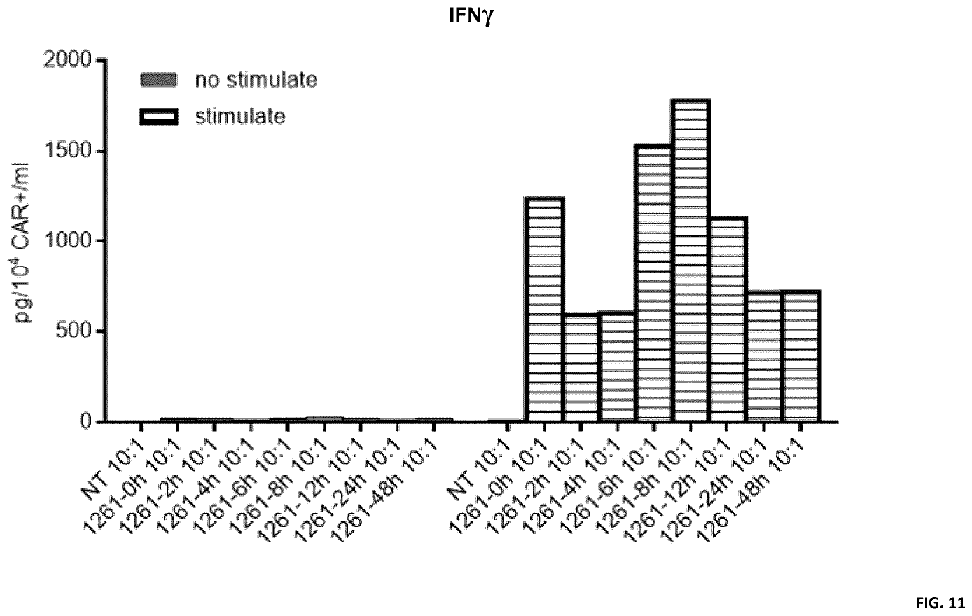

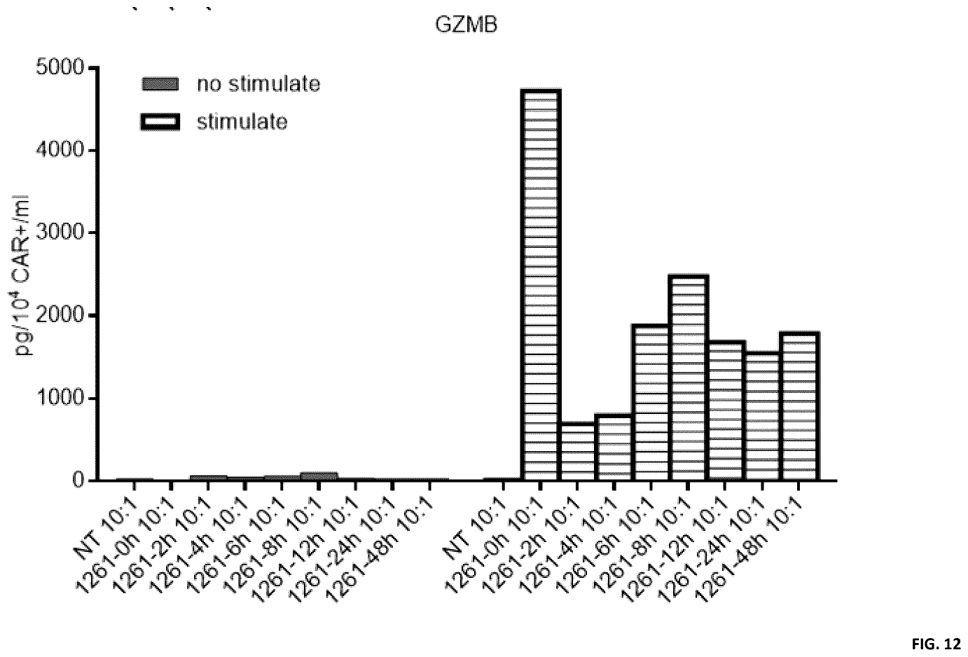

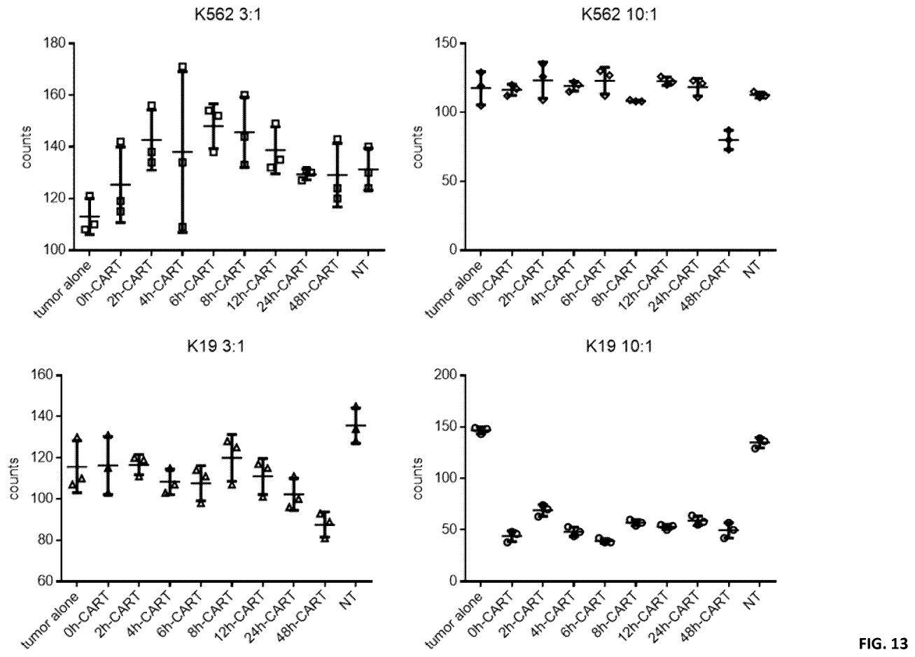

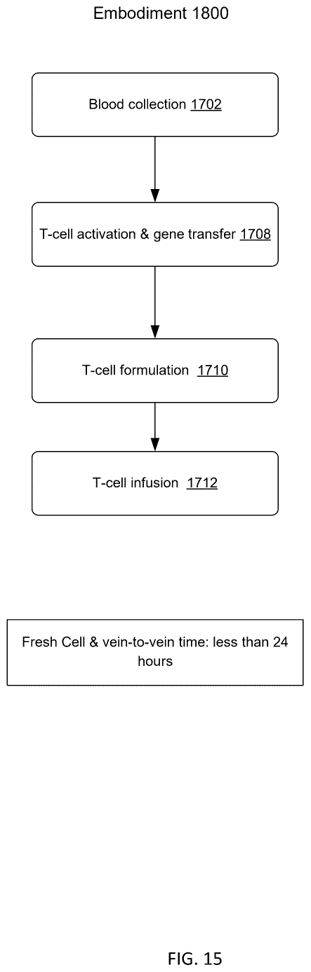

FIG. 3-12 show cytokine release of CD19 CAR T cells cultured with CD19 positive cells for 24 hours. FIG. 13 shows results of functional analysis of CD19 CAR T cells cultured with CD19 positive cells for 24 hours. These data indicate that Embodiments 1400-1800 shown in FIGS. 14 and 15 can be implemented by a closed and/or automatic system, which allows vein-to-vein cell on-site therapy using CAR T cells within 24 hours or even less time without human interferences.

Modified Cells Derived from Blood Cells

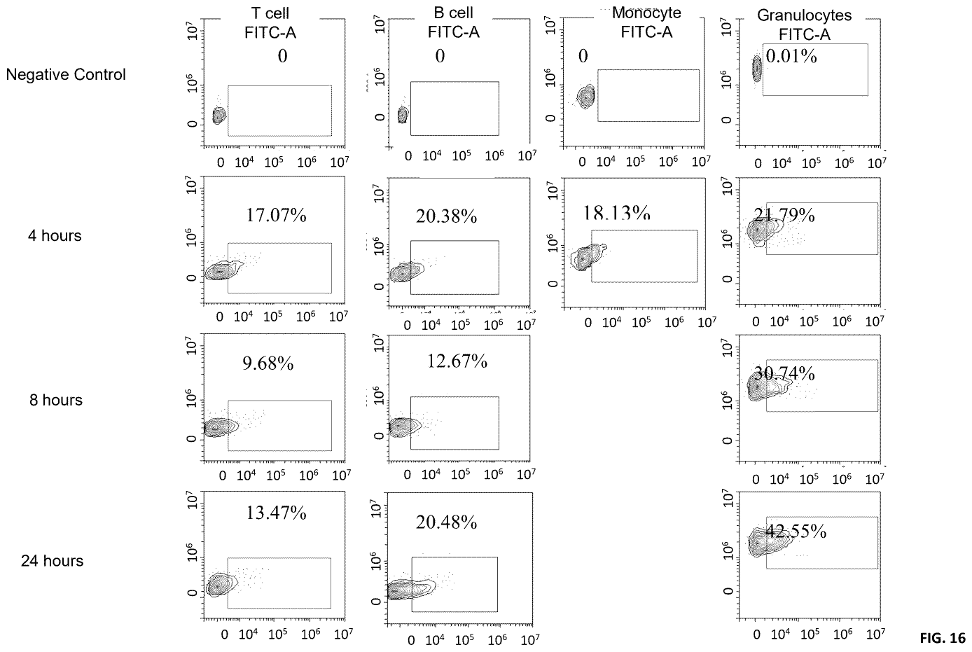

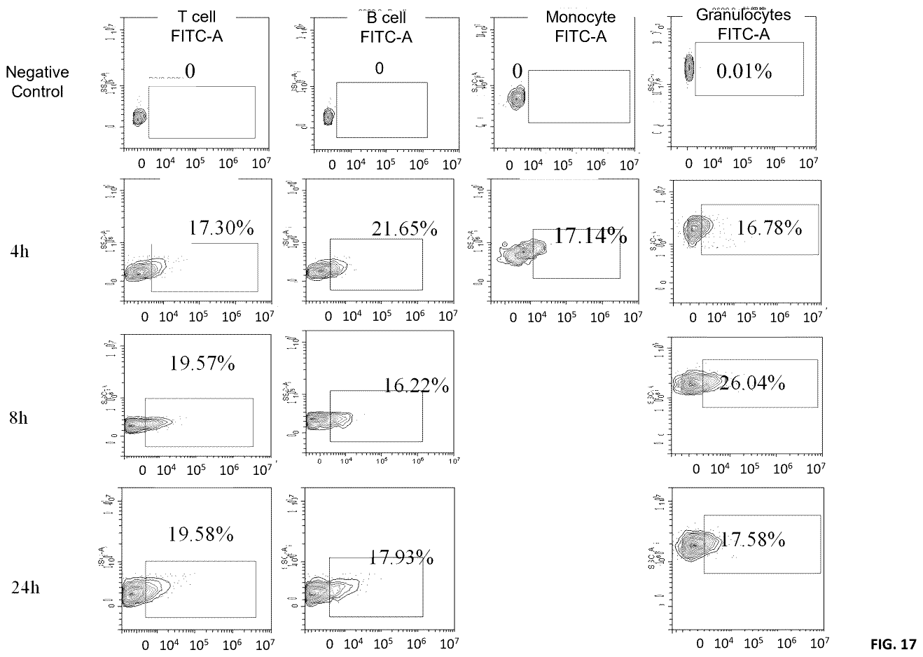

FIG. 16 shows flow cytometry results of GFP expression by blood cells. FIG. 17 shows flow cytometry results of GFP expression by blood cells (Multiplicity of infection (MOI) 400:1) using media including 1% volume of TransAct™. On day 0, human peripheral blood samples (HPBS) were taken and divided into different groups. HPBS were mixed with media containing 1% volume of TransAct™ and lentivirus vectors encoding GFP (MOI 200:1). HPBS were then analyzed using flow cytometry at 4 hours (hrs), 8 hrs, 24 hrs, and 48 hrs after the mixing. As shown in FIGS. 16 and 17, T cells, B cells, monocytes, and granulocytes in the HPBS were successfully transduced with the vectors.

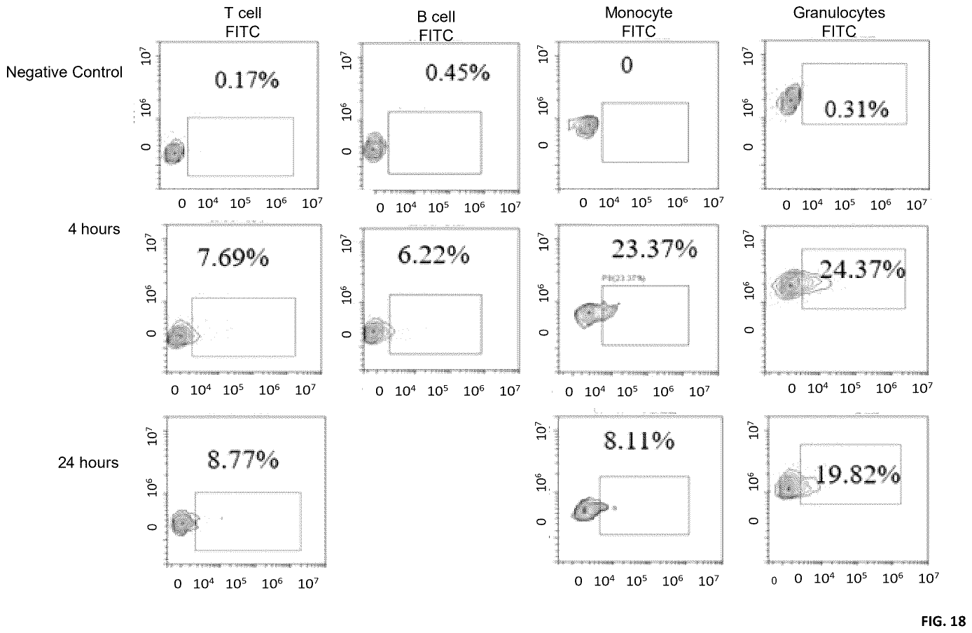

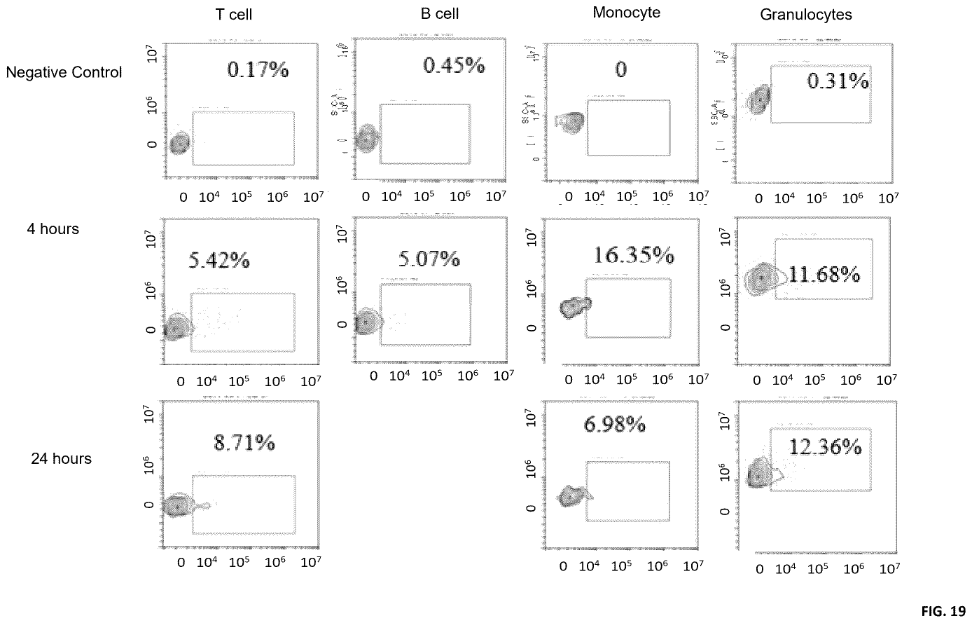

FIG. 18 shows flow cytometry results of GFP expression by blood cells (MOI 200:1) using media without including TransAct™. FIG. 19 shows flow cytometry results of GFP expression by blood cells (MOI 400:1) without media, including CD3/CD28 beads. On day 0, HPBS were taken and divided into different groups. HPBS were mixed with media not containing 1% volume of TransAct™ and lentivirus vectors encoding GFP (MOI 200:1). HPBS were then analyzed using flow cytometry at 4 hrs, 8 hrs, 24 hrs, and 48 hrs after the mixing. As shown in FIGS. 18 and 19, without TransAct™, T cells, B cells, monocytes, and granulocytes in the HPBS were also successfully transduced with the vectors.

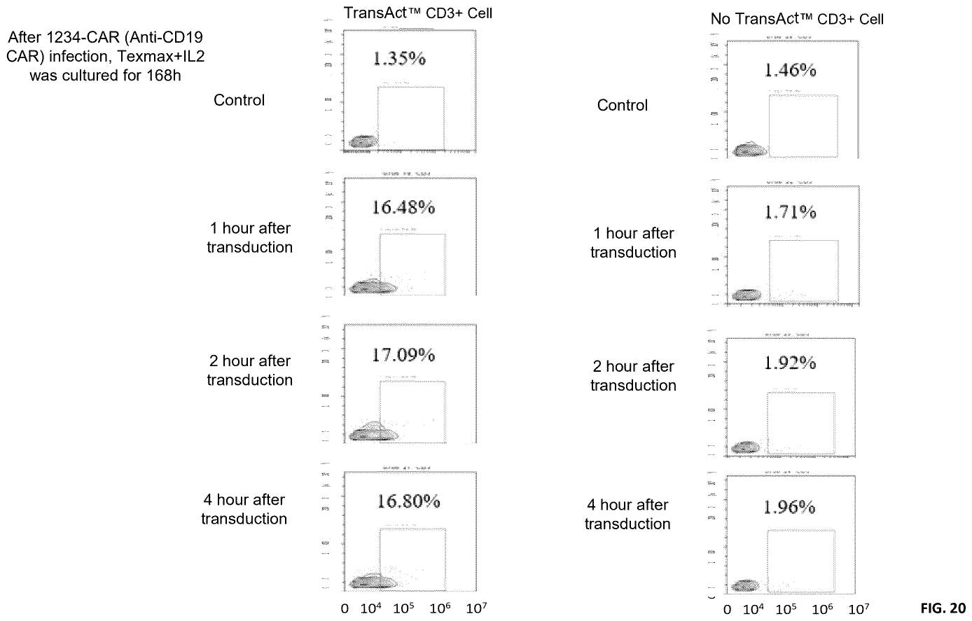

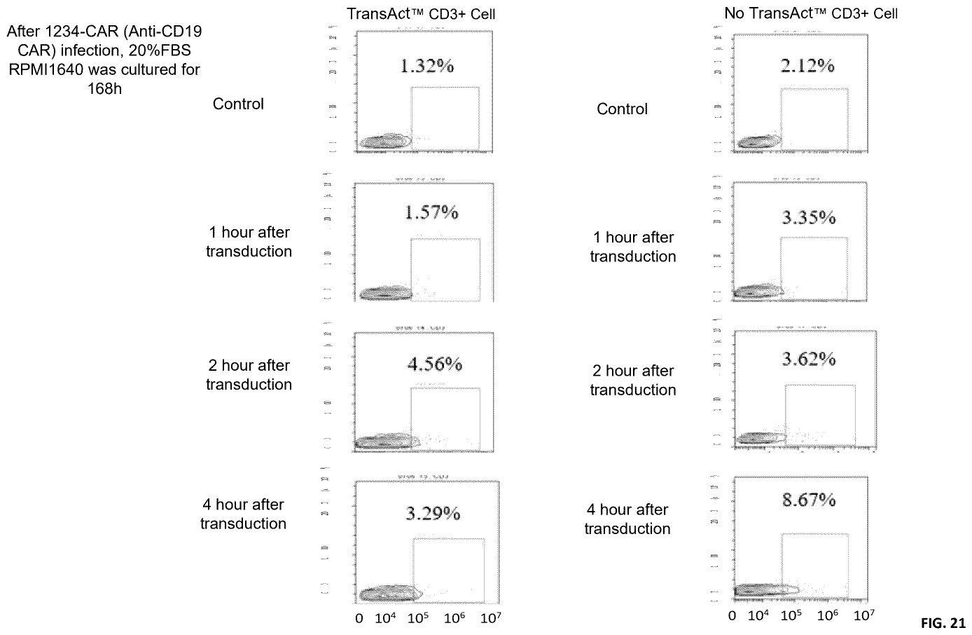

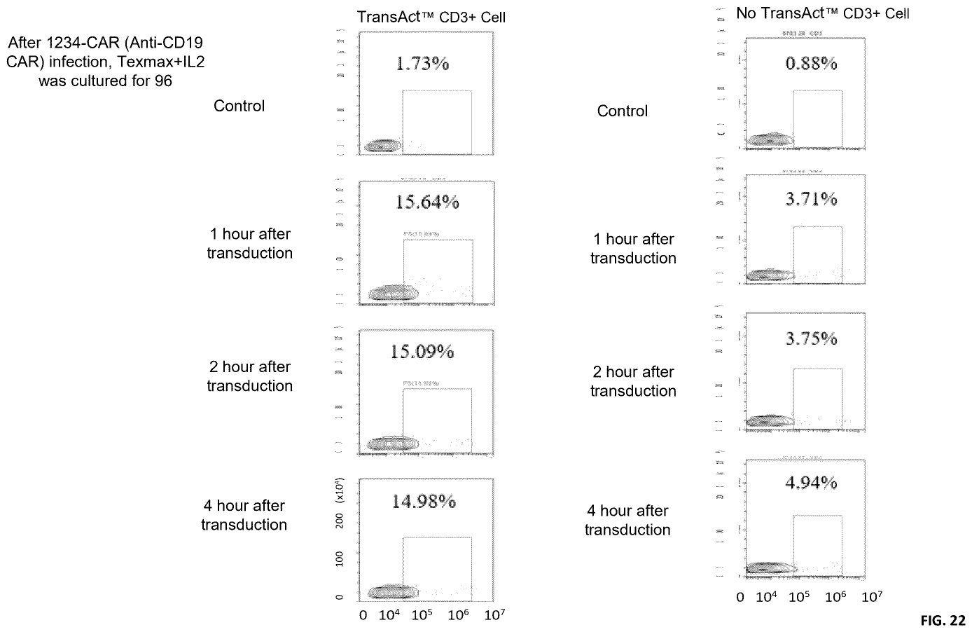

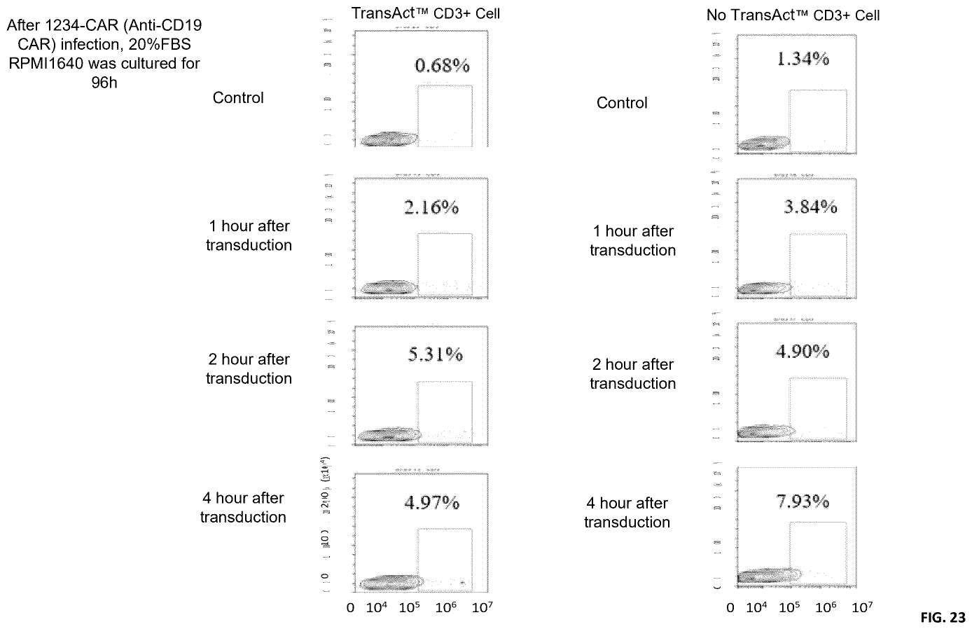

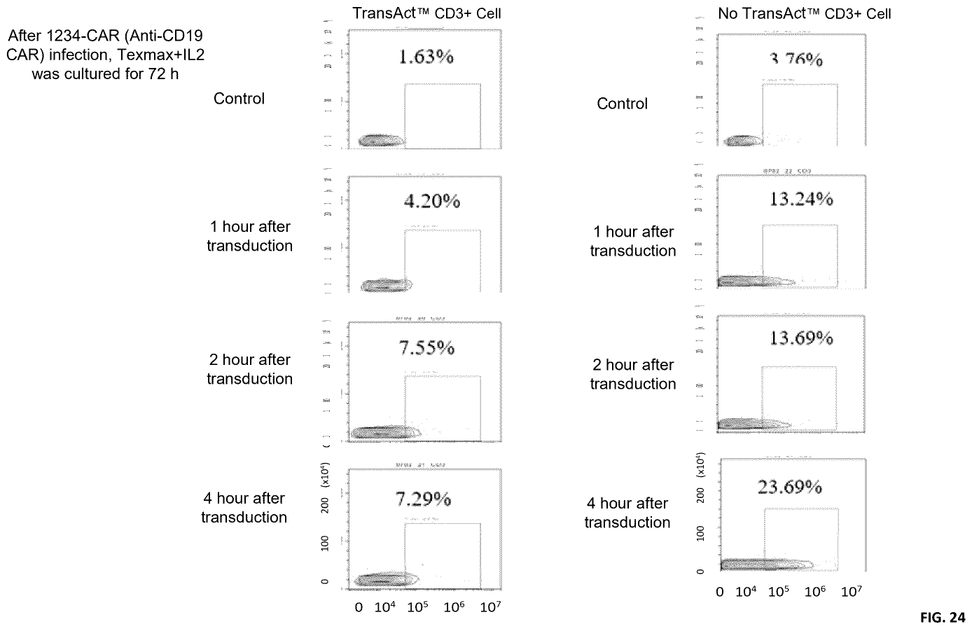

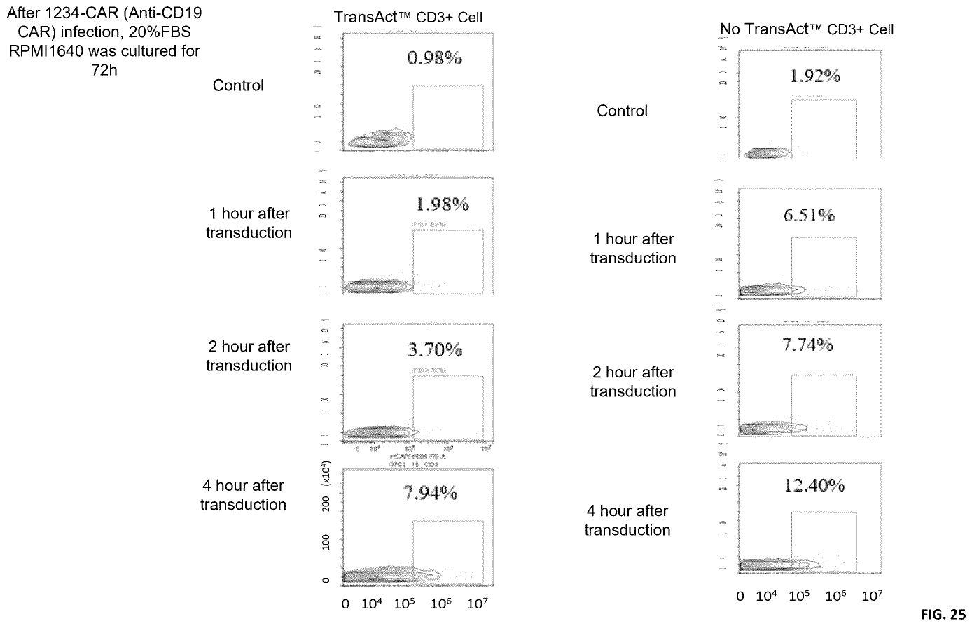

FIGS. 20-25 show flow cytometry results of CAR expression in T cells cultured with various media. On day 0, HPBS were taken and divided into different groups. Some HPBS were mixed with media listed in Tables 5 and 6 and lentivirus vectors (1234) encoding CD19 CAR (MOI 200:1) to obtain CD19 CAR T cells (1234 cells or 1234-CAR cells). Transduction efficiency was measured 7 days after the mixing. As shown in FIG. 20, unlike transduction of vectors encoding GFP, transduction efficiency of CAR was low when the media did not include TransAct™.

TABLE 5

| 1234- | | Incubation | Transfer | |
|---|---|---|---|---|
| CAR | TransAct ™ | Time | agent | Medium |
| MOI | TransAct ™ (1%) | 1 h | PS | 20% FBS-RPMI1640 |
| 200:1 | | 2 h | PS | 20% FBS-RPMI1640 |
| | | 4 h | PS | 20% FBS-RPMI1640 |
| MOI | No TransAct ™ | 1 h | PS | 20% FBS-RPMI1640 |
| 200:1 | | 2 h | PS | 20% FBS-RPMI1640 |
| | | 4 h | PS | 20% FBS-RPMI1640 |
| MOI 0 | TransAct ™ (1%) | 4 h | PS | 20% FBS-RPMI1640 |
| MOI 0 | No TransAct ™ | 4 h | PS | 20% FBS-RPMI1640 |

TABLE 6

| 1234- | | Incubation | T ransfer | |
|---|---|---|---|---|
| CAR | TransAct ™ | Time | agent | Medium |
| MOI | TransAct ™ (1%) | 1 h | PS | TexMACS ® + IL2 |
| 200:1 | | 2 h | PS | TexMACS ® + IL2 |
| | | 4 h | PS | TexMACS ® + IL2 |
| MOI | No TransAct ™ | 1 h | PS | TexMACS ® + IL2 |
| 200:1 | | 2 h | PS | TexMACS ® + IL2 |
| | | 4 h | PS | TexMACS ® + IL2 |
| MOI 0 | TransAct ™ (1%) | 4 h | PS | TexMACS ® + IL2 |
| MOI 0 | No TransAct ™ | 4 h | PS | TexMACS ® + IL2 |

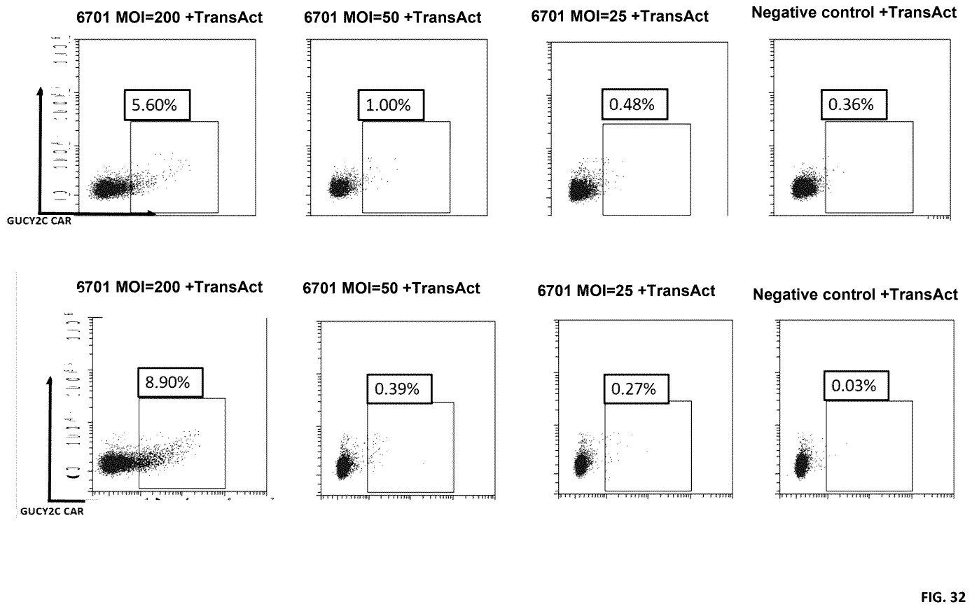

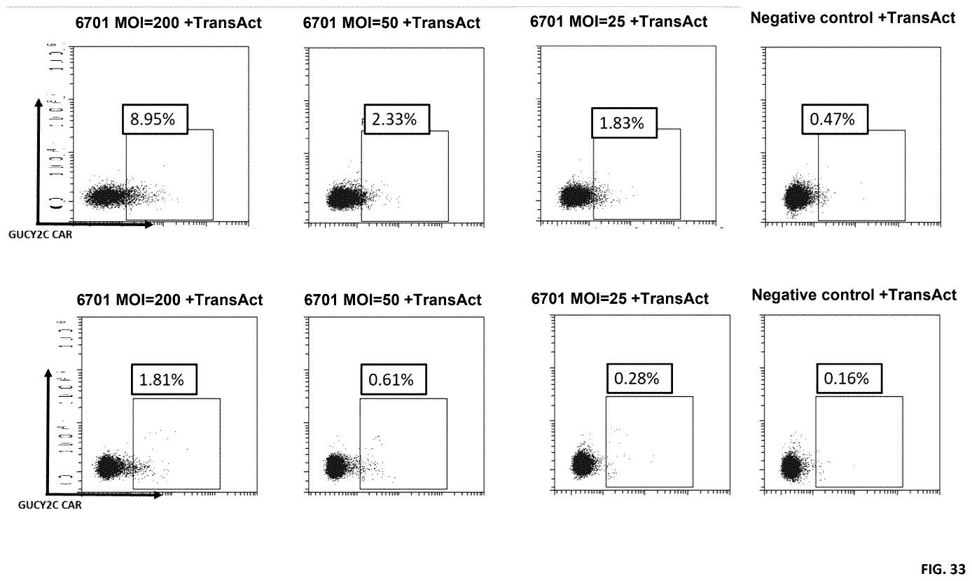

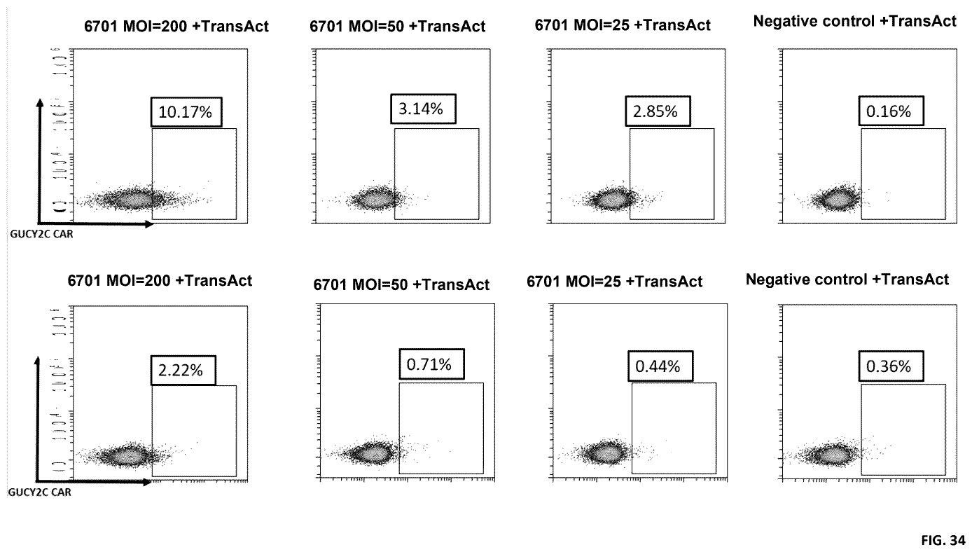

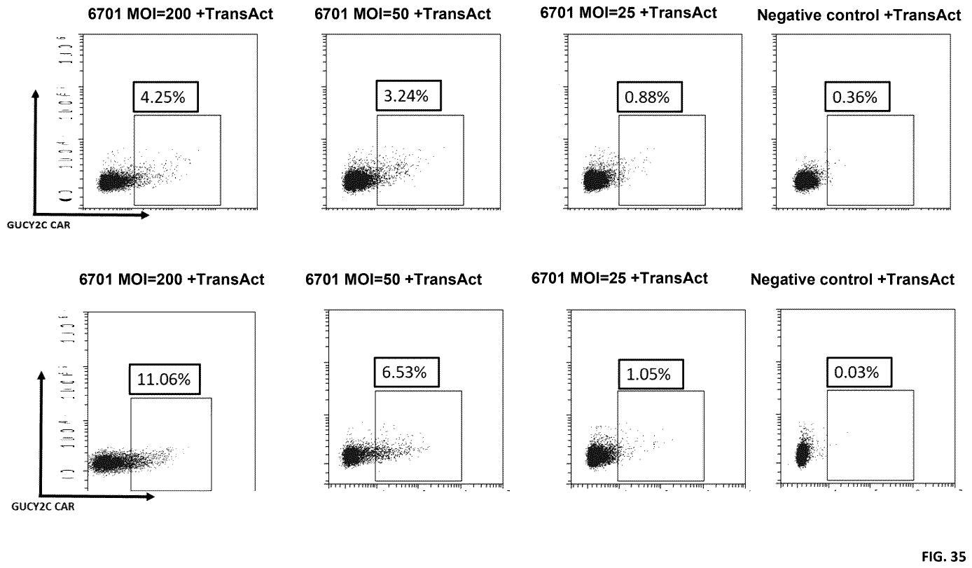

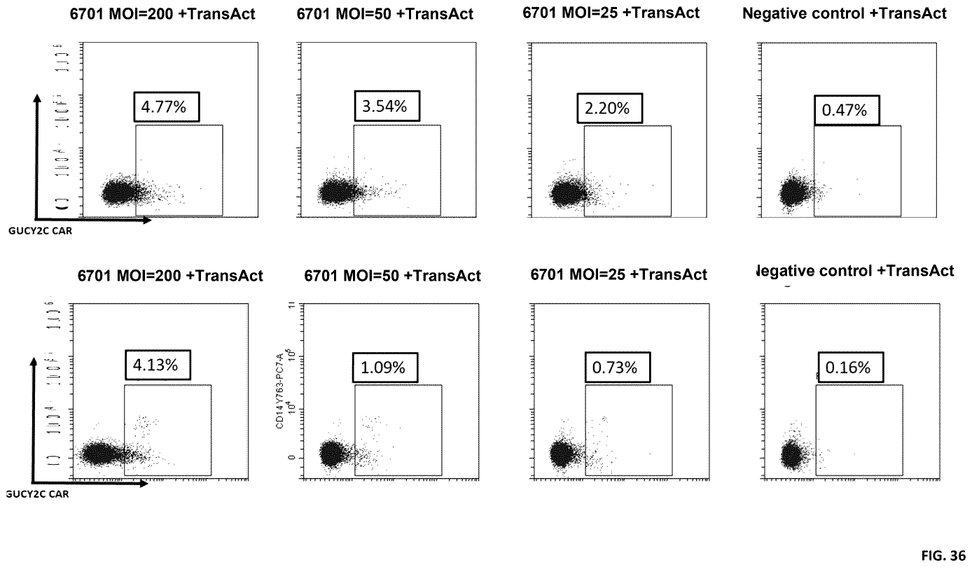

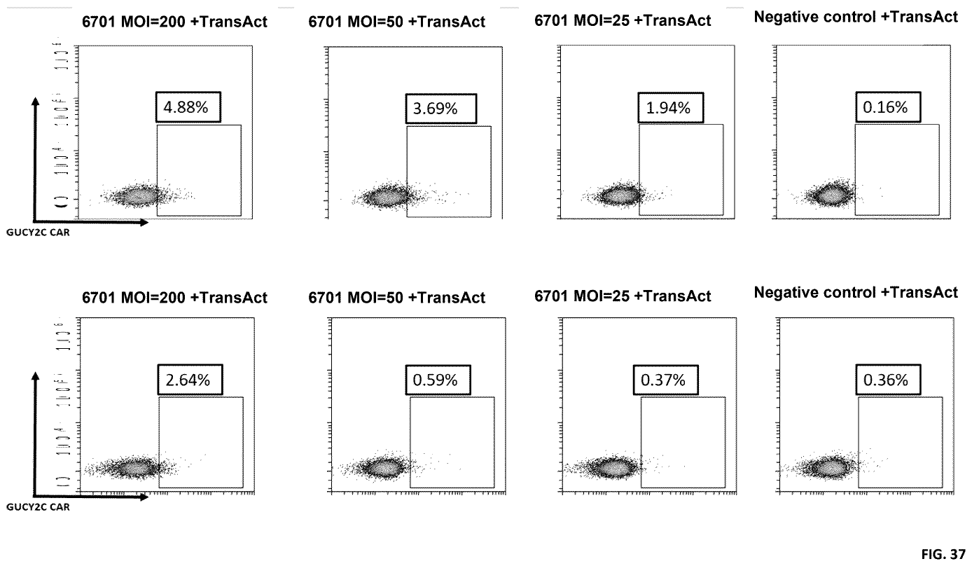

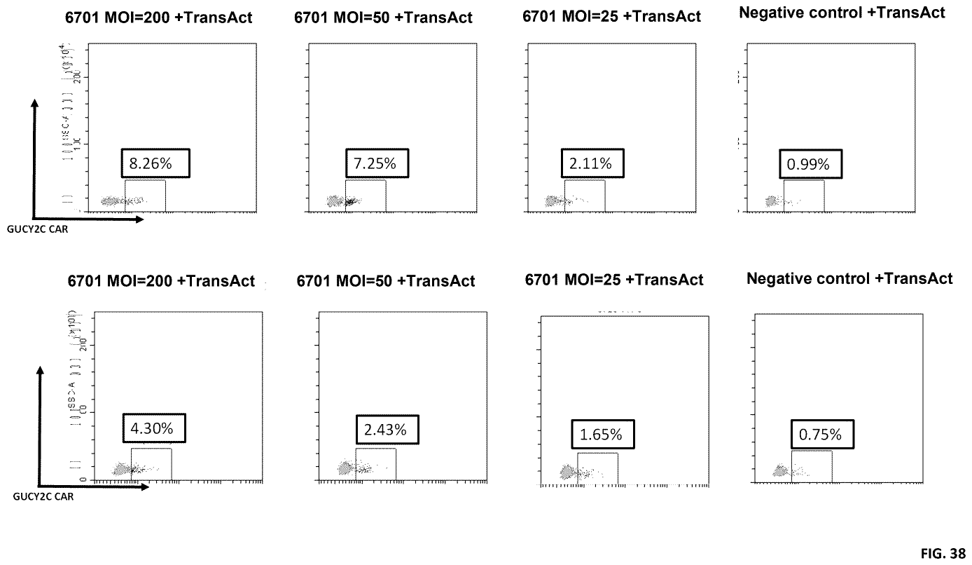

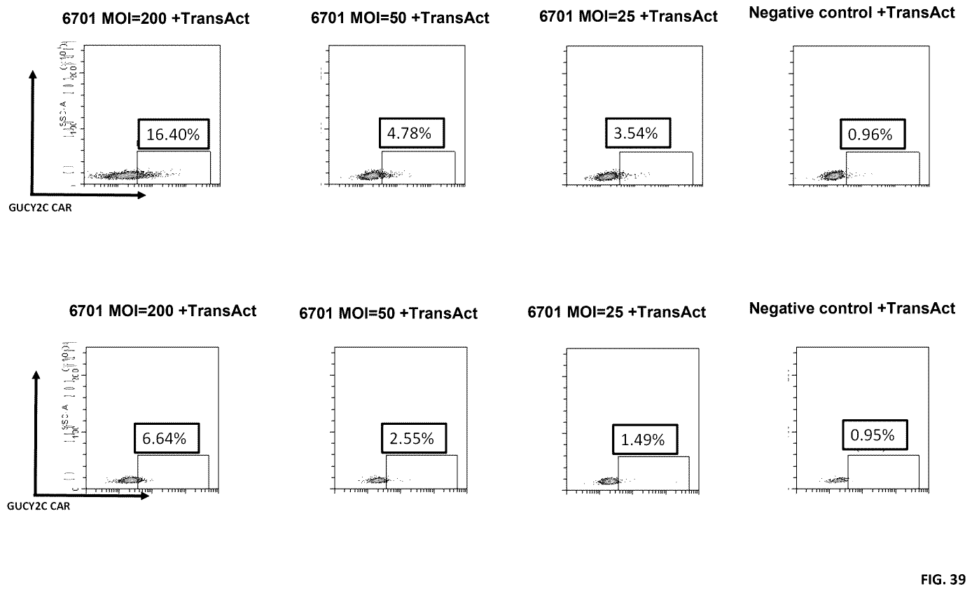

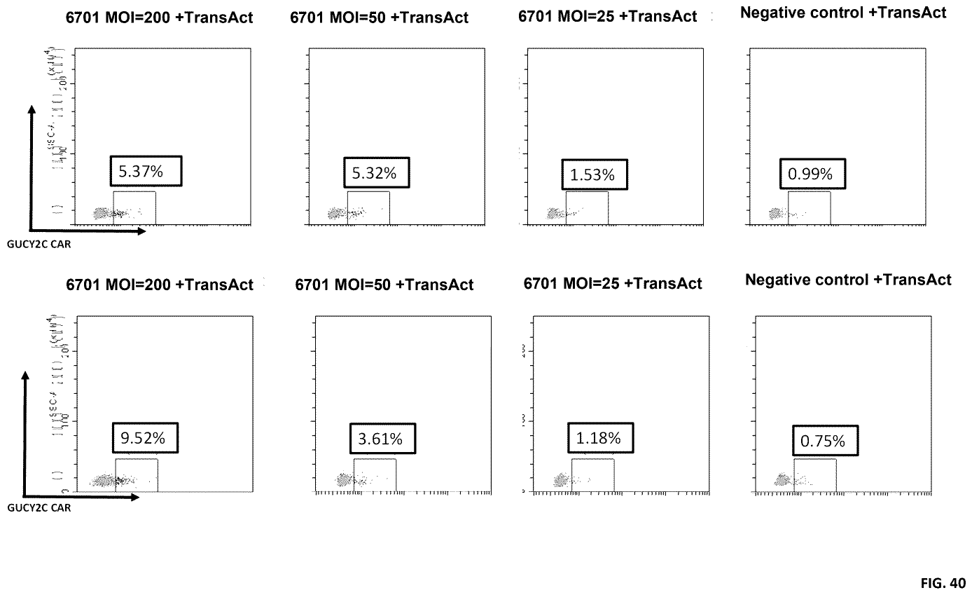

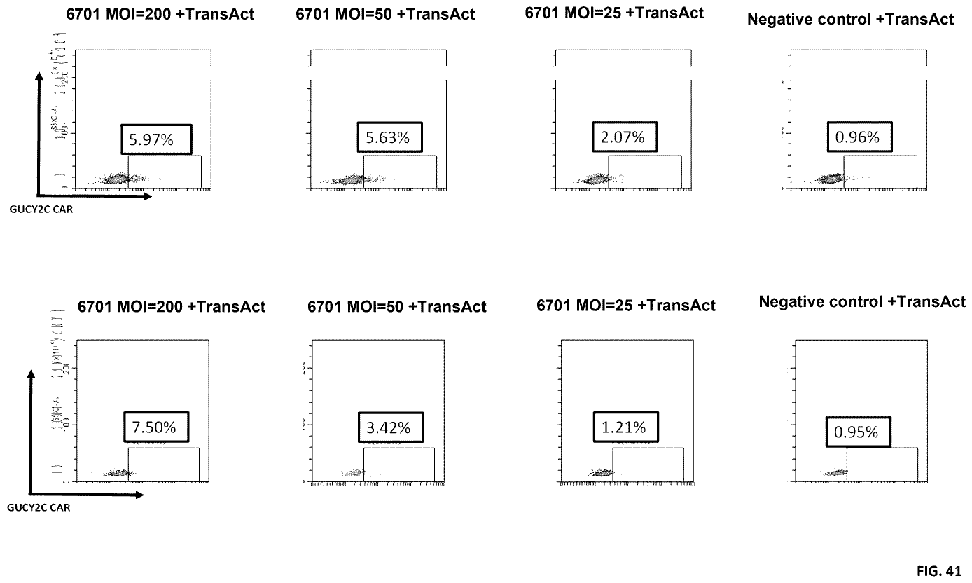

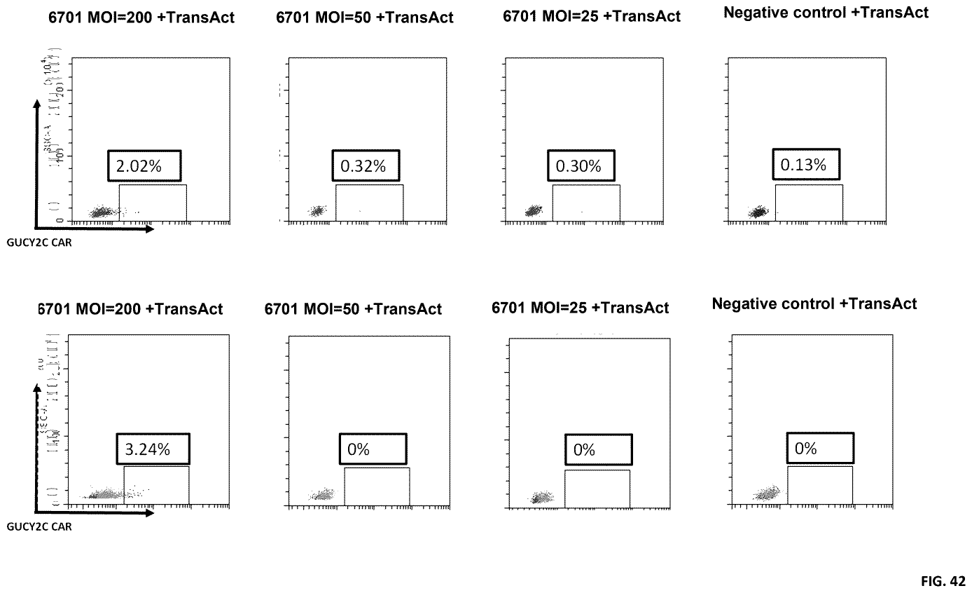

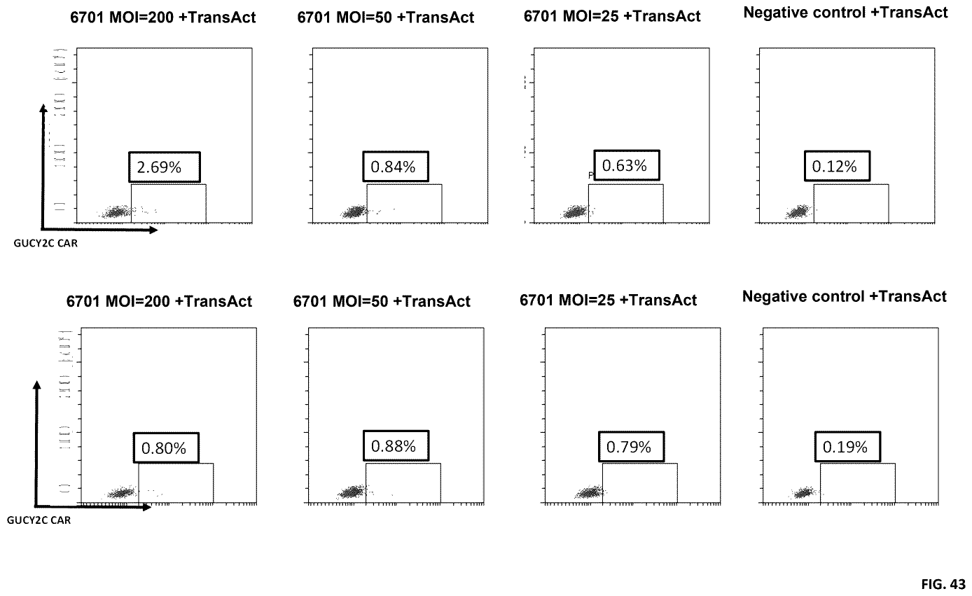

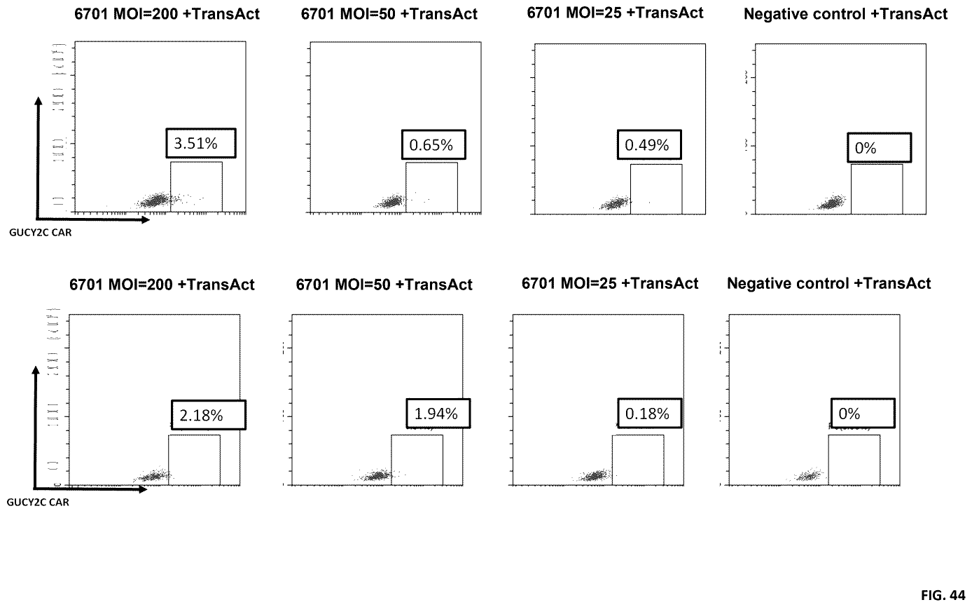

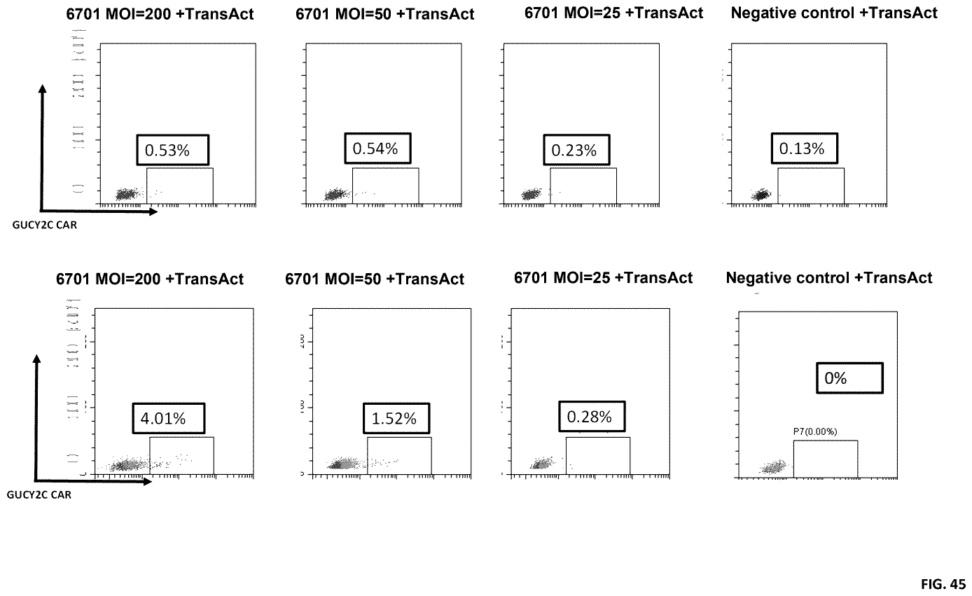

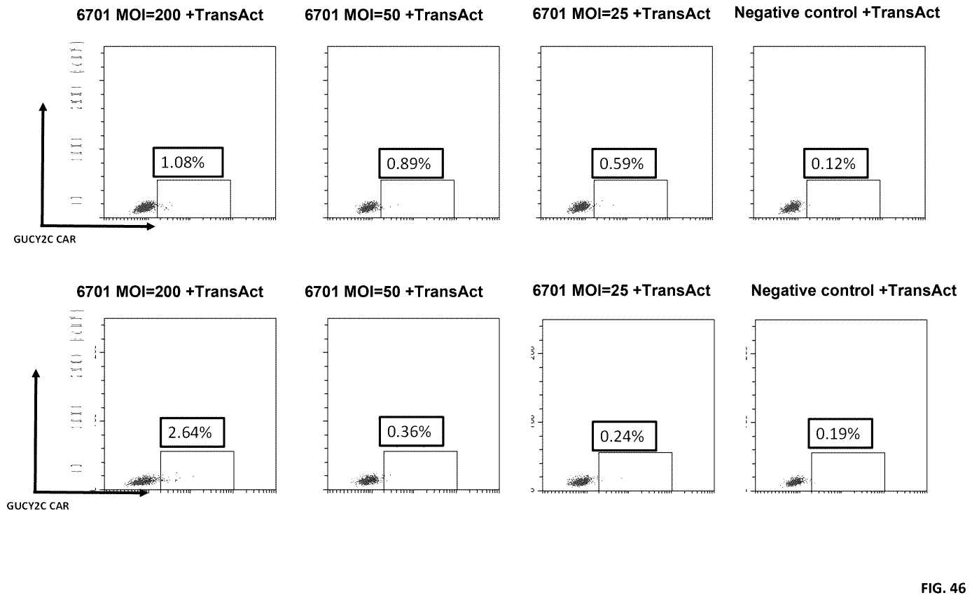

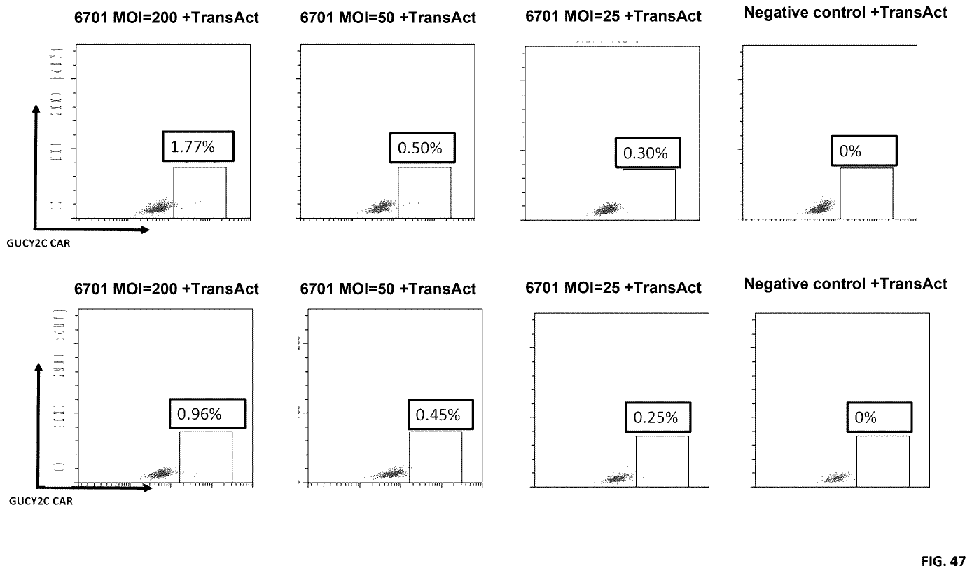

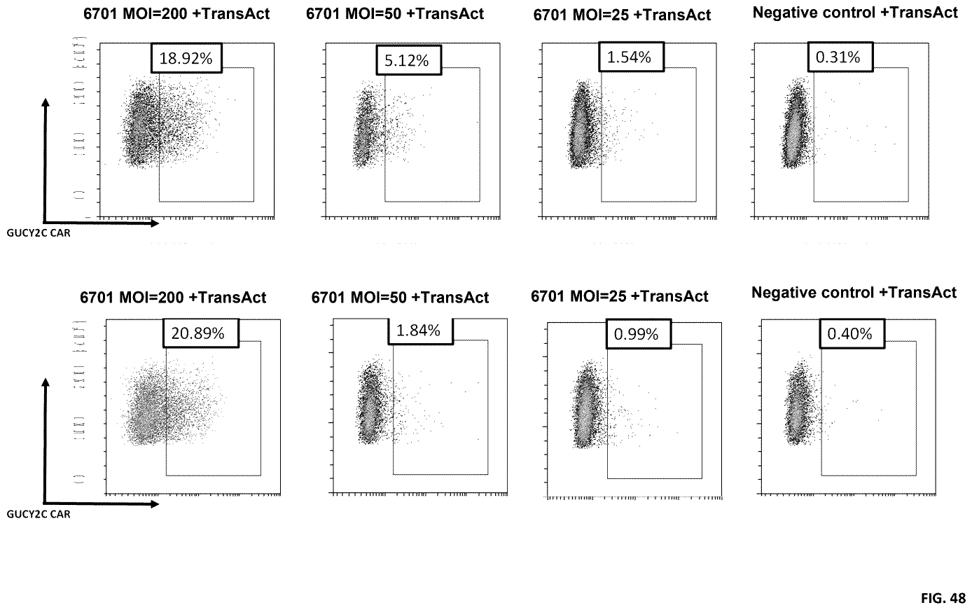

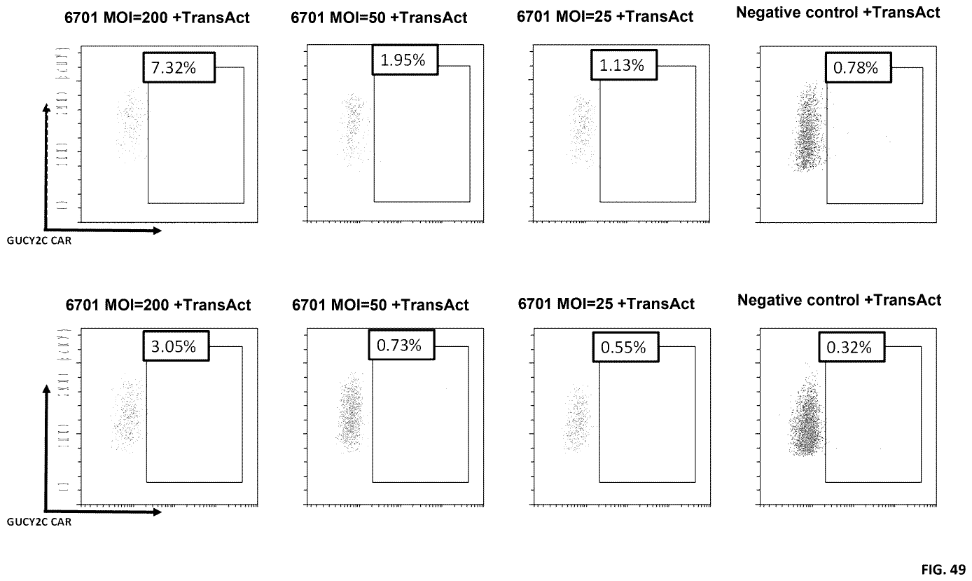

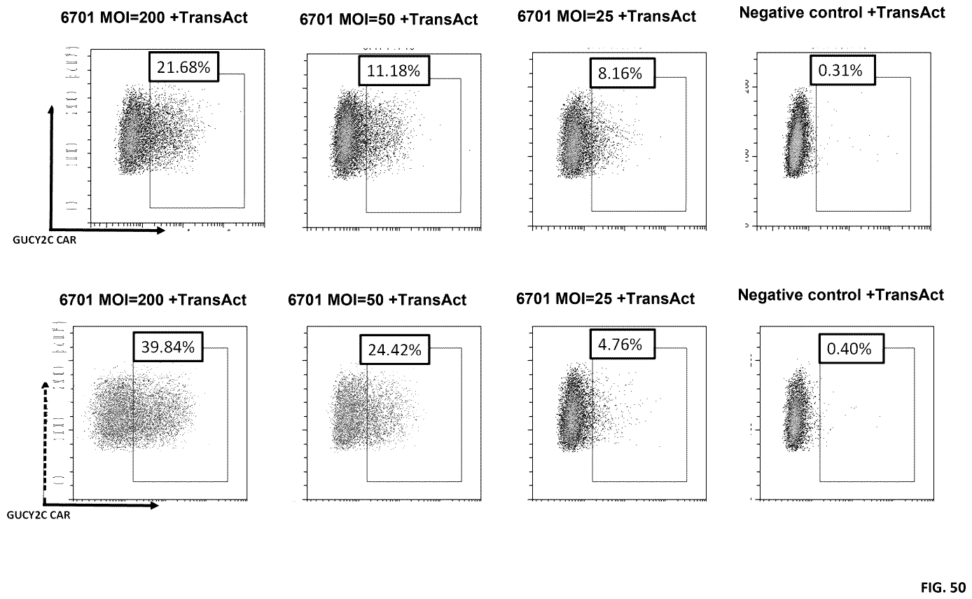

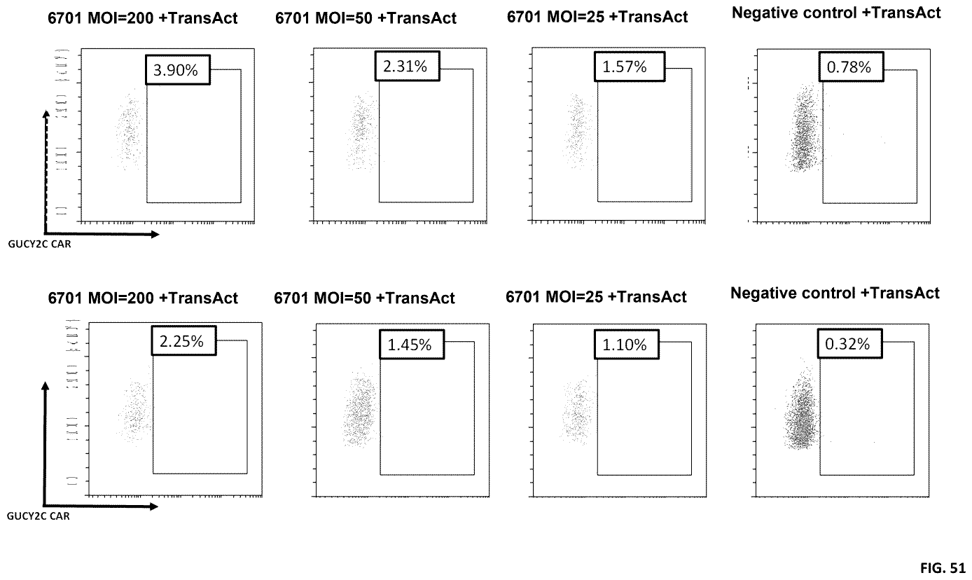

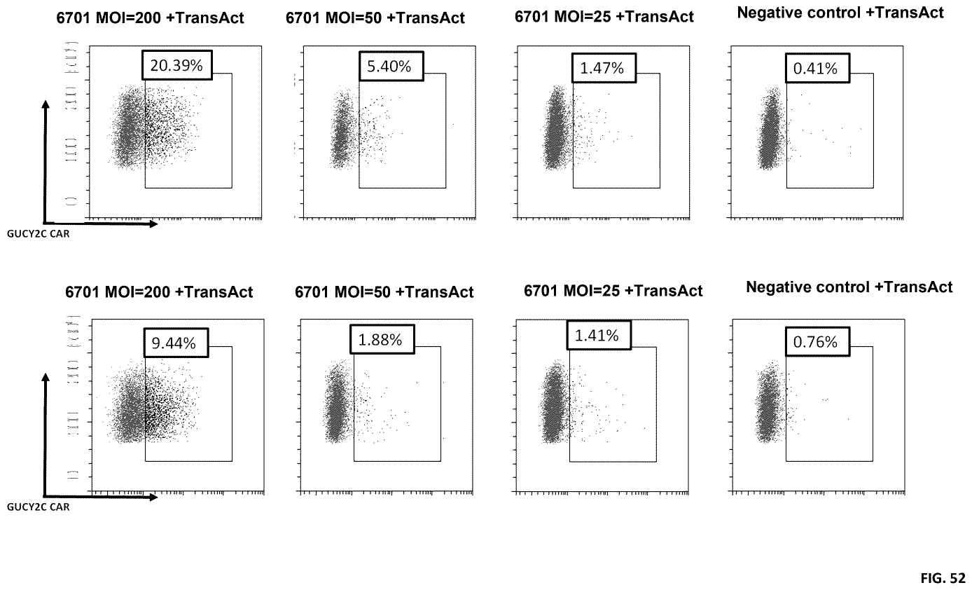

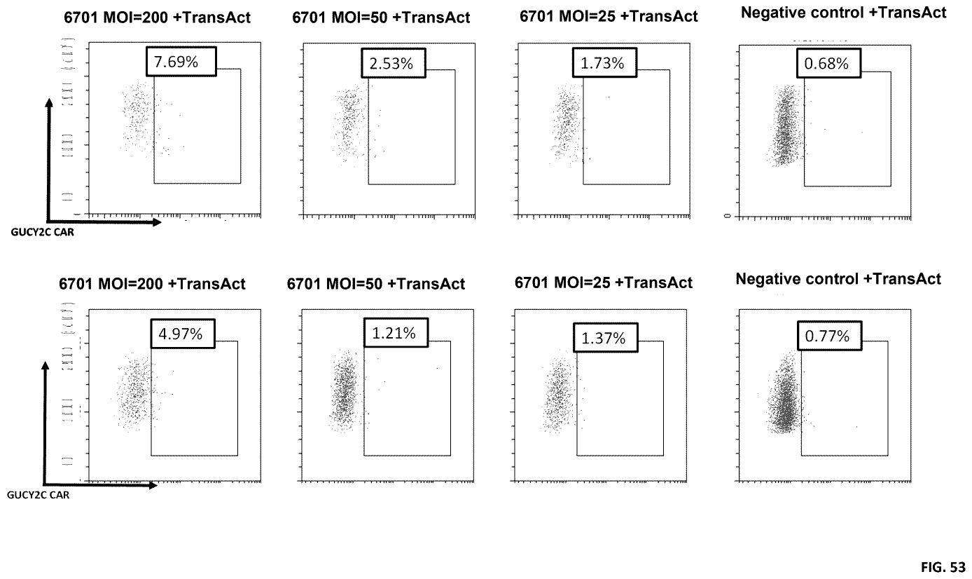

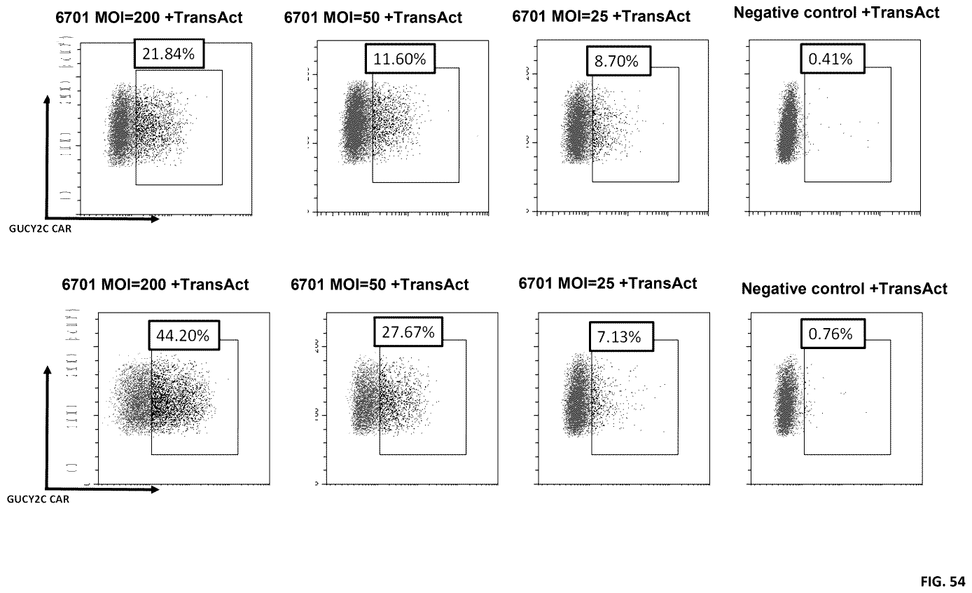

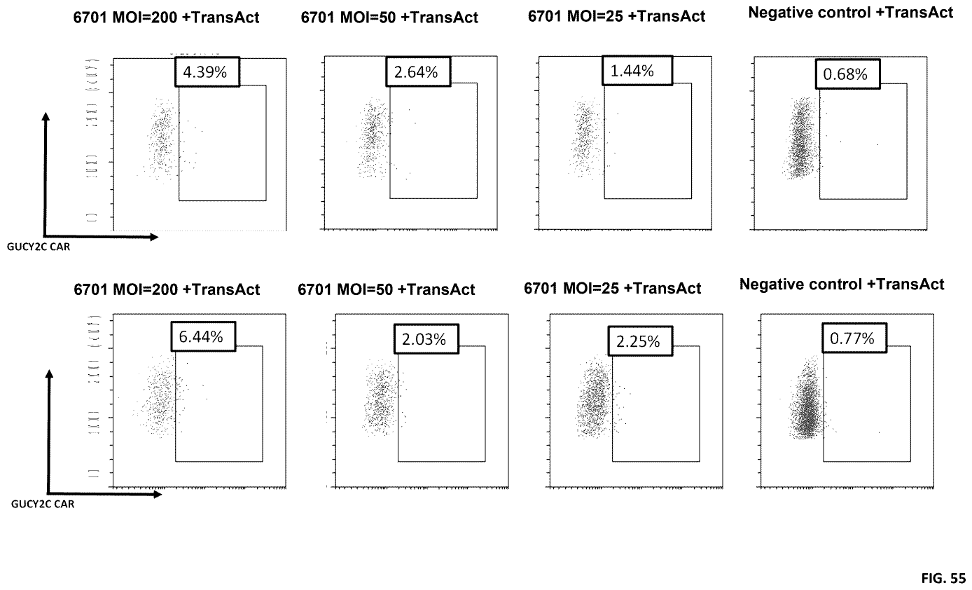

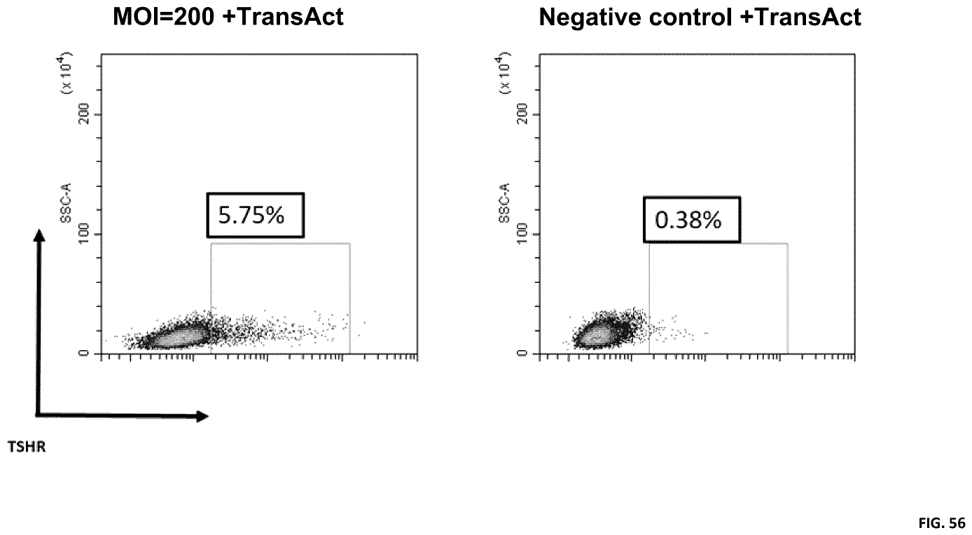

FIGS. 32-56 show flow cytometry assay results of whole blood transduction of vectors encoding GUCY2C CAR (i.e., 6701) in various examples. On day 0, HPBS were obtained from volunteers and mixed with lentivirus vectors encoding GUCY2C CAR in media with or without TransAct™ using different MOIs (200, 50, and 25:1) for 2 or 4 hours. Twenty-four hours and forty-eight hours after mixing, human peripheral blood cells (HPBC) were washed to remove the nontransduced vectors and the TransAct™. On day 2, 5, and 6, a flow cytometry assay was performed to measure the expression of GUCY2C CAR on cells such as T cells, NK cells, granulocytes, monocytes, and B cells.

As shown in FIGS. 32-56, the expression of GUCY2C CAR on T cells, NK cells, granulocytes, monocytes, and B cells appeared to be different. TransAct™ enhanced the expression of GUCY2C CAR on T cells as compared to other cell types. In other words, vectors appeared to infect more T cells than other cell types in the media containing TransAct™.

These data confirmed that whole blood transfection is feasible to implement the gene and cellular therapy system described herein. For example, T cells, NK cells, granulocytes, monocytes, and B cells can all be transfected. These data also indicated that the media including TransAct™ and low MOI (e.g., less than 200) caused high expression of CAR on T cells and low expression on B cells.

### Gene/Cellular Therapy System and In Vivo Use Thereof

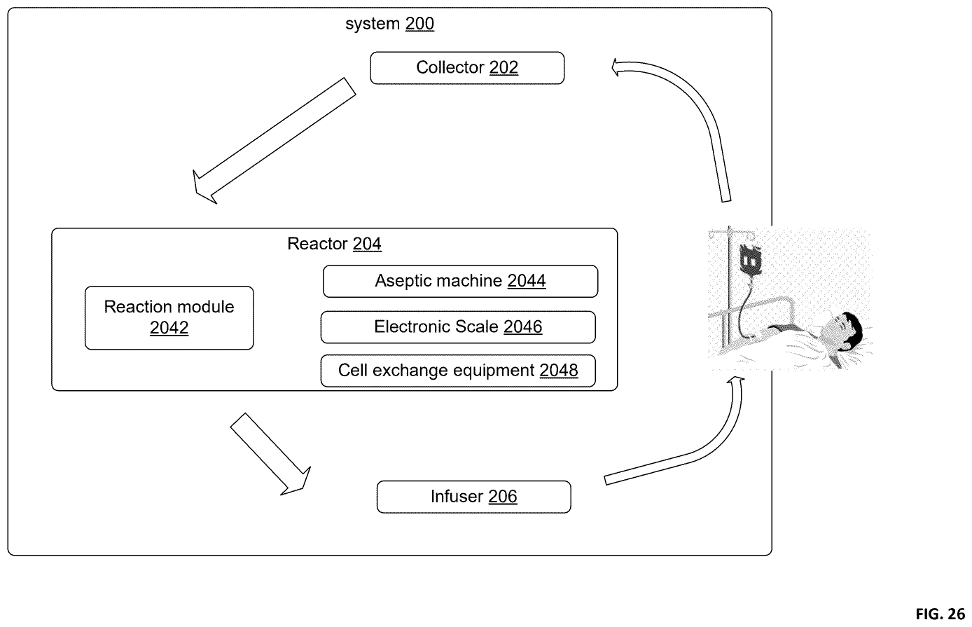

FIG. 26 shows a schematic view of a gene/cell therapy system 200, which can include additional modules, lines, and devices. The system 200 can include collector 202, reactor 204, and infuser 206. Collector 202 is configured to collect peripheral blood from a patient's vein. For example, collector 202 can be a blood collection bag, which is sterile and includes a blood collection needle, tube, blood collection bag, and containing anticoagulant.

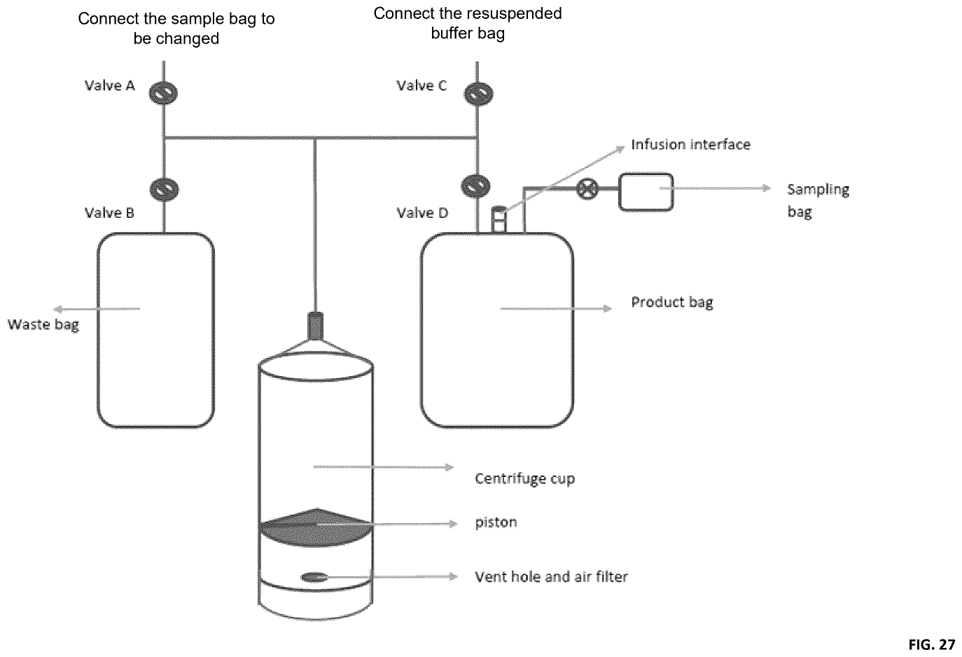

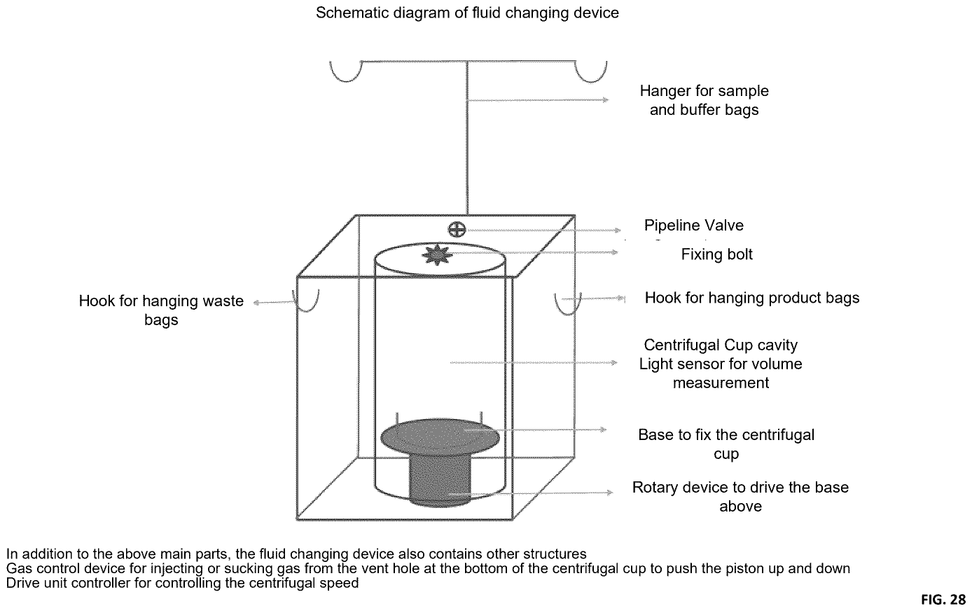

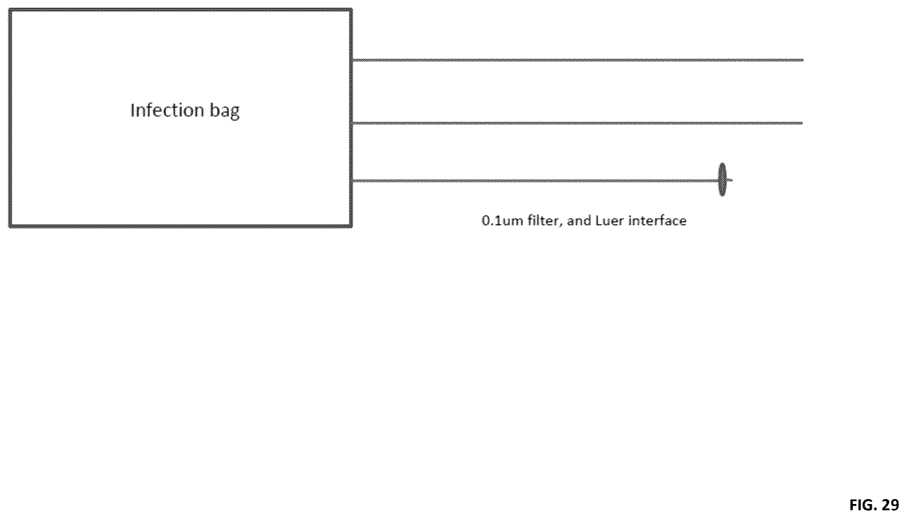

Reactor 2042 can include reaction module 2042, aseptic takeover machine 2044, electronic scale 2046, and cell exchange equipment 2048. Reactor 204 can be configured to activate blood cells collected from the vein and introduce targeting vectors into the blood cells. Reactor 204 can include an infection bag configured to mix reagents and blood for introducing the vectors into the blood cells. For example, the infection bag can be sterile and include three lines, one of which is connected to a 0.1 μm filter with a Luer interface. Reactor 204 can further include a fluid changing module configured to change the medium after the infection is completed, remove the viral vector, and replace the buffer. For example, the fluid changing module can be sterile, a centrifuge cup connected with a waste fluid bag, a product bag, and two fluid lines used to connect the post-infection sample and the buffer for reinfusion. The centrifugal cup contains a piston, and there is a vent hole under the centrifugal cup. The vent hole can suck in or expel gas through the air filter. The centrifugal cup has a slot, which can be fixed on the fluid changing device to keep the cup stable during the centrifugation process. Reactor 204 can further include an infusion buffer bag, a viral solution bag, and activation reagents. The infusion buffer bag can be configured to wash cells to obtain products that can be returned. For example, the infusion buffer bag can be sterile and equipped with a buffer that can be returned. The viral solution bag can be configured to save infected virus vectors, can be sterile and include a virus vector, and can be connected to the infection bag. The activation reagent can be configured to activate cells and can be sterile and added to the infection bag through the filter via a syringe. FIG. 27 shows a schematic view of the fluid change line module. FIG. 28 shows a schematic view of the fluid changing device described in FIG. 27. FIG. 29 shows a schematic view of the infection bag.

An aseptic machine 2044 can be configured to aseptically join lines in common areas. An electronic scale can be configured to quantify liquid. Cell exchange equipment can be configured to be used in conjunction with the fluid change line module. The cell suspension is changed and resuspended with the prepared buffer to output the product and maintain the aseptic condition of the cell sample during the process.

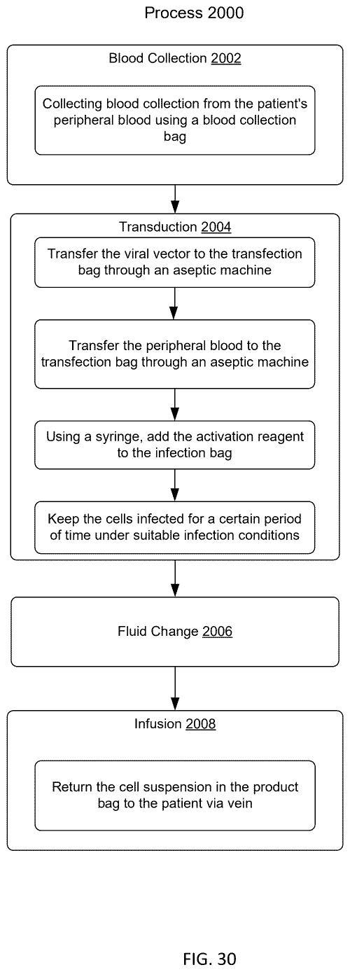

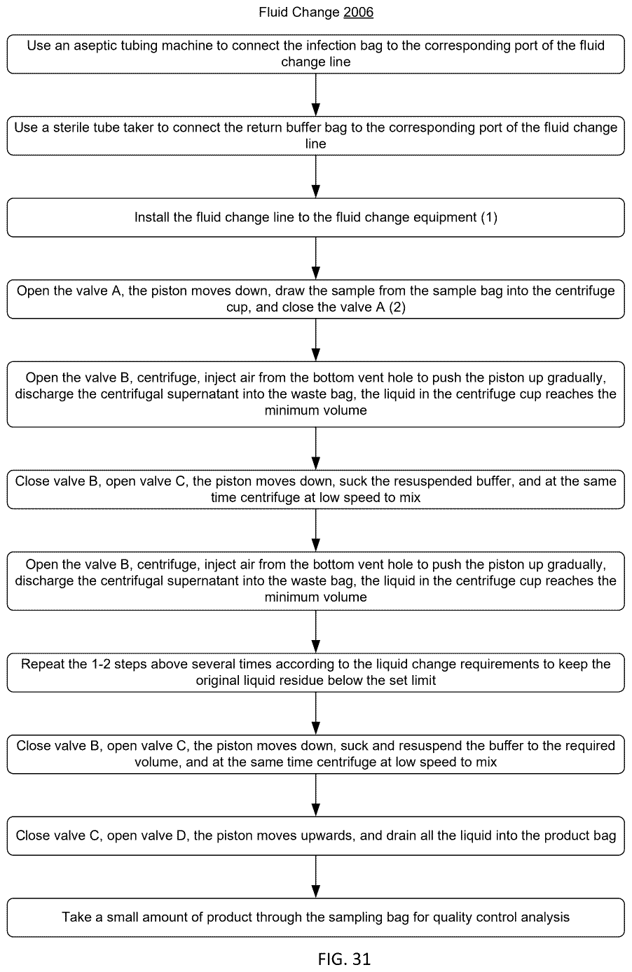

FIGS. 30 and 31 show flow diagrams of an illustrative process 2000 for implementing a cell and/or gene therapy system. Process 2000 is illustrated as a collection of blocks in a logical flow graph, which represents a sequence of operations that can be implemented in hardware, software, or a combination thereof. The order in which the operations are described is not intended to be construed as a limitation, and any number of the described blocks can be combined in any order and/or in parallel to implement the process. Process 2000 is described with reference to system 200, as well as those described in FIGS. 26-29. However, process 2000 can also be implemented in other environments and/or with other equipment.

In embodiments, blood can be collected and obtained from the patient's peripheral blood using a blood collection bag. The viral vector transferred to the transfection bag through an aseptic machine, and the peripheral blood moved to the infection bag through an aseptic machine. Physicians or other trained personnel can use a syringe to add the activation reagent to the infection bag to keep the cells infected for a certain period of time under suitable conditions; can use an aseptic tubing to connect the infection bag to the corresponding port of the fluid change line; and can use a sterile tube to connect the return buffer bag to the corresponding port of the fluid change line. As shown in FIG. 27, system 200 connects the fluid change line to the fluid change equipment (1) and open valve A. The piston moves down (2), and system 200 draws the sample from the sample bag into the centrifuge cup, and closes valve A, opens valve B, centrifuges, injects air from the bottom vent hole to push the piston up gradually, to discharge the centrifugal supernatant into the waste bag. The fluid in the centrifuge cup reaches the minimum volume, and system 200 closes valve B and opens valve C. The piston moves down, and system 200 sucks the resuspended buffer, mixes at the same time, centrifuges at low speed, opens the valve B, centrifuge, injects air from the bottom vent hole to push the piston up gradually, to discharge the centrifugal supernatant into the waste bag. The fluid in the centrifuge cup reaches the minimum volume, and system 200 repeats the steps above several times according to the requirements for changing fluids to keep the original residual fluid below the set limit, close valve B, and open valve C. The piston moves down, and system 200 sucks and resuspend the buffer to the required volume, mixes at the same time, centrifuges at low speed, closes valve C, and opens valve D. The piston moves upwards, and system 200 drains all the fluid into the product bag, takes a small amount of the product through the sampling bag for quality control analysis, and returns the cell suspension in the product bag to the patient via the vein.

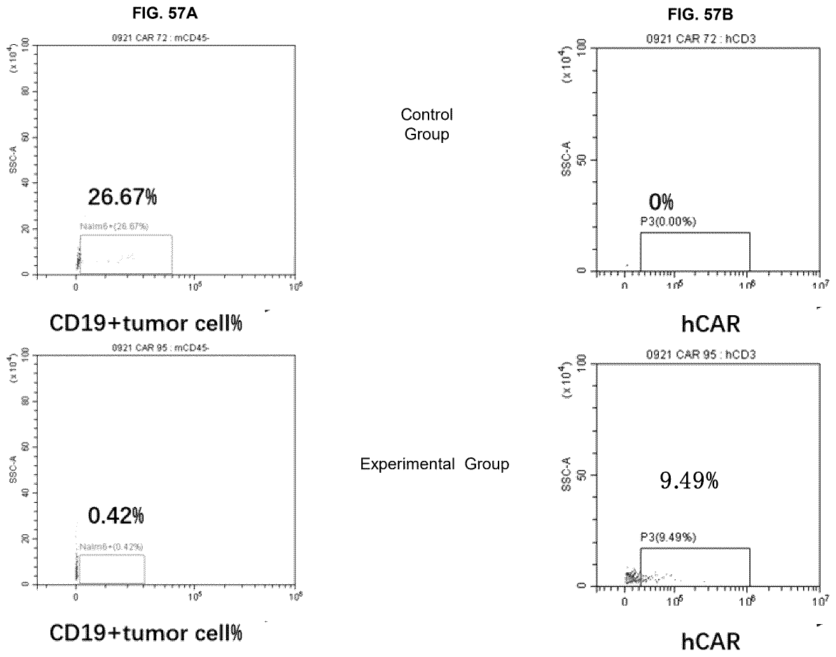

The system was tested using animal models. Heterotransplantation of human cancer cells or tumor biopsies into immunodeficient rodents (xenograft models) has, for the past two decades, constituted the major preclinical screen for the development of novel cancer therapeutics (Song et al., Cancer Res. PMC 2014 Aug. 21, and Morton et al., Nature Protocols, 2, -247-250 (2007)). To evaluate the anti-tumor activity of CART T cells in vivo, immunodeficient mice bearing tumor xenografts were used to evaluate CAR T cells' antitumor activity. CD19+ tumor cells (Nalm6 cells) were used to establish the immunodeficient mice bearing CD19 tumor xenografts. On day 1, Nalm6 cells were injected into the tail veins of the immunodeficient mice. On day 2 or 3, the immunodeficient mice were irradiated in 2 Gy fractions. On day 3, tumor cells were formed in the immunodeficient mice w. Mice were divided into two groups. For the control group, uninfected blood excluding red blood cells was used. For the experimental group, blood excluding red blood cells was transduced according to the protocol described above for whole blood transfection. FIG. 57 shows flow cytometry results of in vivo whole blood transfection. FIG. 57 A shows flow cytometry results that only 0.42% of the tumor cells were left in mice transfused with CD19 CAR transduced whole blood cells, while 26.27% of the tumor cells were left in the mice of the control group, demonstrating that CD19 CAR transduced whole blood cells can kill CD19+ tumor cells.

### Enhancement of Gene Therapy

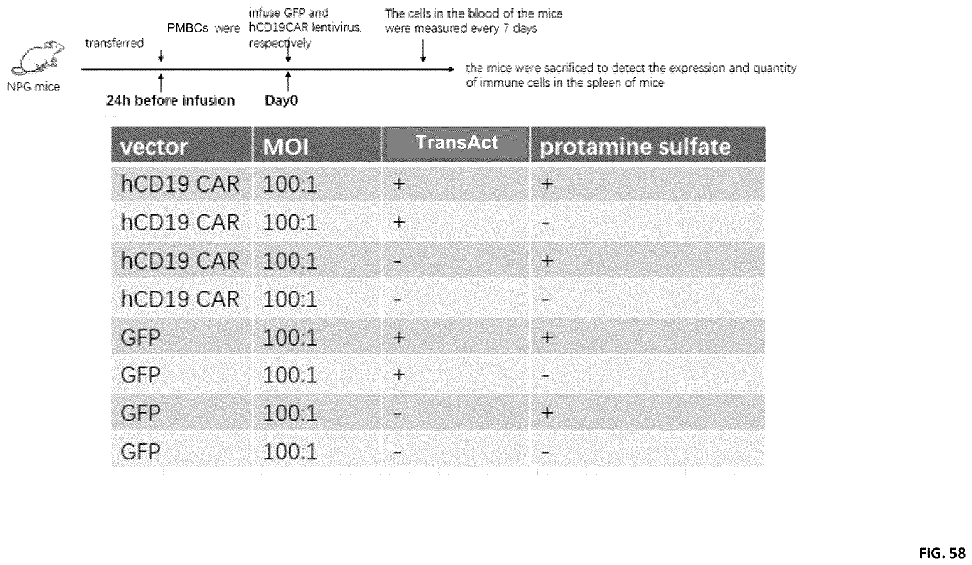

FIG. 58 shows a scheme of in vivo examples. As shown in FIG. 58, human whole blood or PBMC was infused into mice, and then lentivirus was administered to the mice to simulate and infect human blood cells. As shown in the Examples above, the lentivirus can infect human T cells or other immune cells by directly injecting into humans with acceptable expression ratios. Twenty-four hours before virus injection, peripheral blood was collected from healthy volunteers, and 1 ml of PBMC was obtained after whole blood sorting was transferred to mice. On day 0, lentiviral vector infusion was carried out according to the scheme shown in FIG. 58 with an estimated average of 106 normal CD3+ cells in PBMCs. Finally, the mice were sacrificed to examine their spleen cells.

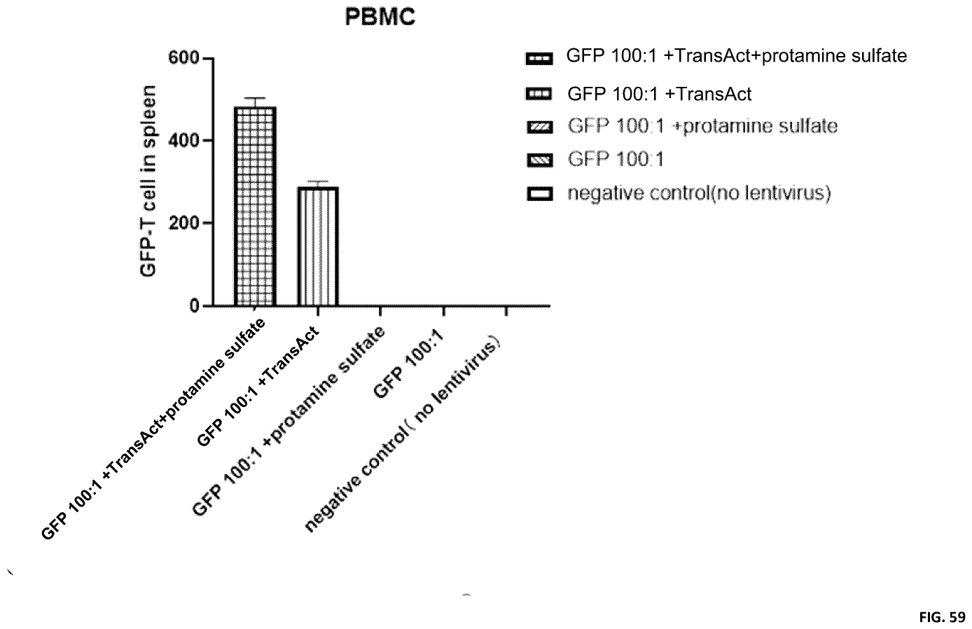

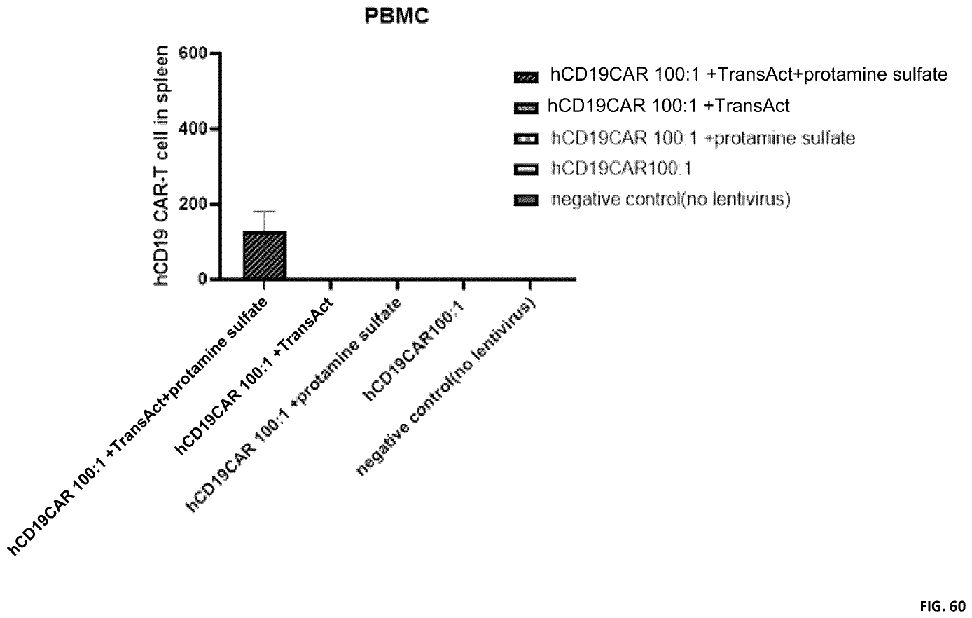

FIG. 59 shows the proportion of GFP-infected CD3+ cells in the spleens of mice that were infused with PBMCs and then infused with lentivirus vectors encoding GFP. As shown in FIG. 59, the infection rate of GFP was significantly increased in the group of cells mixed with TransACT™, and protamine sulfate further enhanced the infection rate of GFP. FIG. 60 shows results of CD19 CAR cell infection in the spleen of mice that were infused with PBMCs and then infused with lentivirus vectors encoding CD19 CAR. As shown in FIG. 60, the infection rate of CD19 CAR was significantly increased in mice infused with vectors mixed with TransACT™ and protamine sulfate. These in vivo results demonstrate that targeting vectors (e.g., lentivirus vectors) encoding a CAR molecule can be introduced to T cells by directly delivering the vectors to mice in the presence of a T cell activator and a transduction adjuvant (e.g., TransACT™ and protamine sulfate).

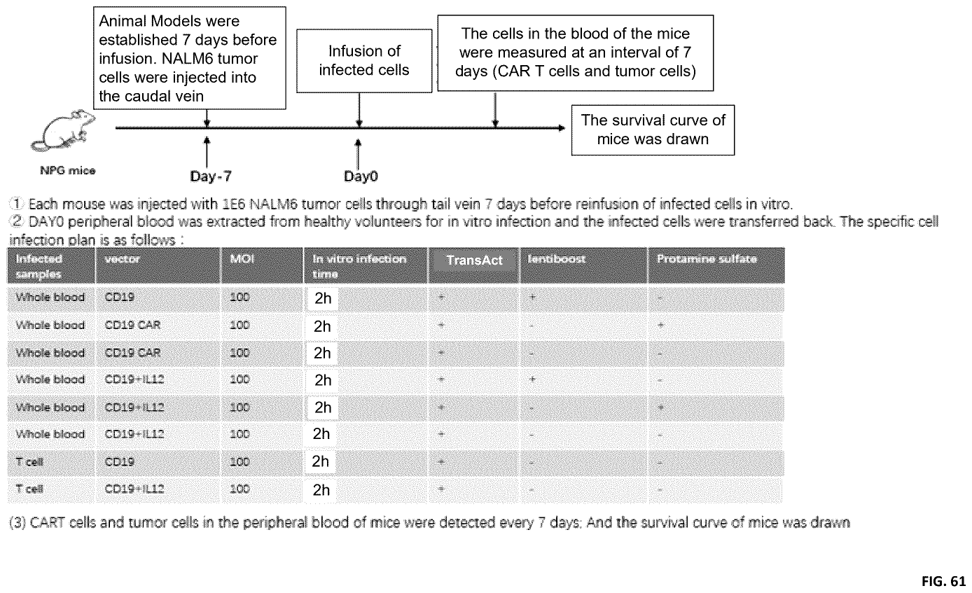

FIG. 61 shows a scheme for the examples related to whole blood transfection. Each mouse was injected with 1×106 NALM6 tumor cells through the tail vein 7 days before reinfusion of infected cells. On day 0, peripheral blood was extracted from healthy volunteers, introduced with lentivirus vectors encoding CD19 CAR and IL-12 to obtain infected cells, and the infected cells were transferred to mice. CAR T cells and tumor cells in the peripheral blood of mice were detected every 7 days, and the survival curve of mice was measured.

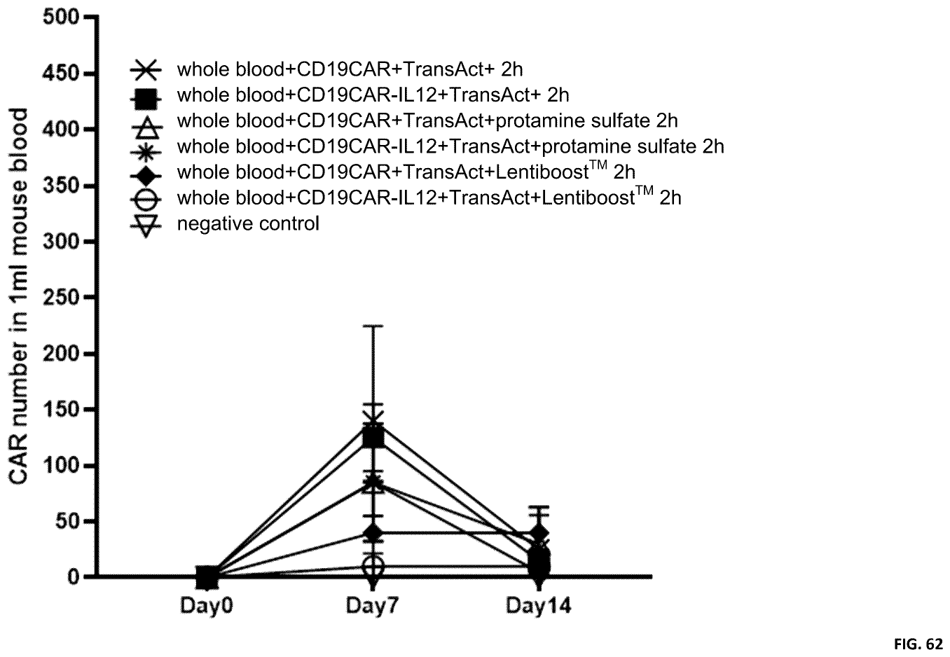

FIG. 62 shows the proliferation of CAR T cells in the peripheral blood of mice after the infected whole blood was infused into the mice. These results indicate that the addition of protamine sulfate (PS) and LentiBOOST™ did not change the proliferation of CAR T cells in mice, in the early stage of cell expansion. On day 14, cell proliferation in peripheral blood of mice in all groups was reduced to a similar level, which can be caused by cell migration to lymphatic organs and killing tumors in vivo.

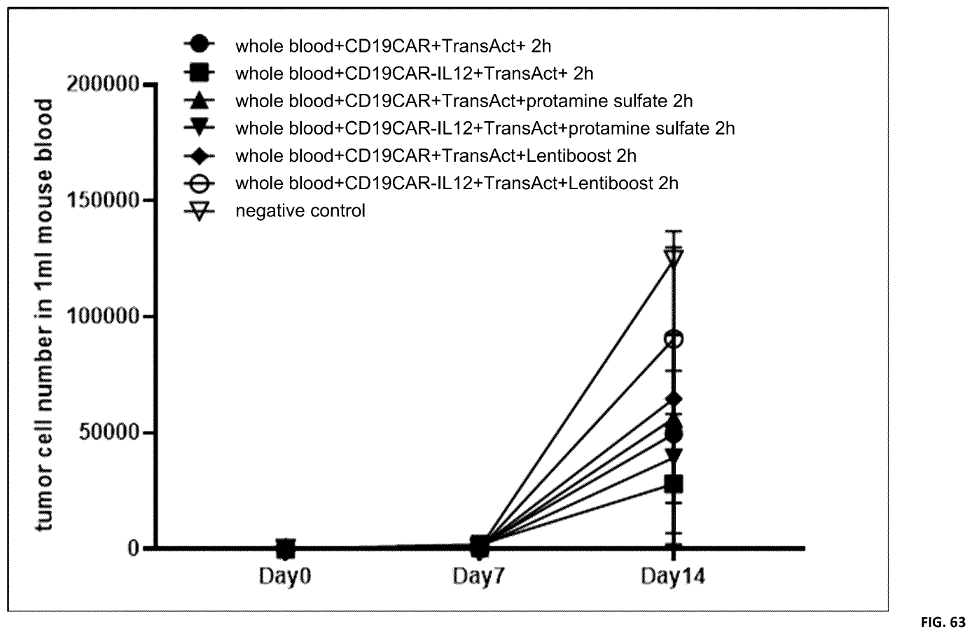

FIG. 63 shows the proliferation of tumor-bearing cells in the peripheral blood of mice after the infected whole blood was infused into the mice. As shown in FIG. 67, the infected whole blood cells inhibited the proliferation of tumor-bearing cells, and it appeared that those cultured with PS or LentiBOOST™ did not inhibit the proliferation of tumor-bearing cells better than those cultured without PS or LentiBOOST™. The infected whole blood cells cultured with TransACT™ and introduced with vectors encoding IL-12 showed better anti-tumor activities than the other groups. Also, the infected whole blood cells introduced with vectors encoding IL-12 showed better anti-tumor activities than those cells without IL-12.

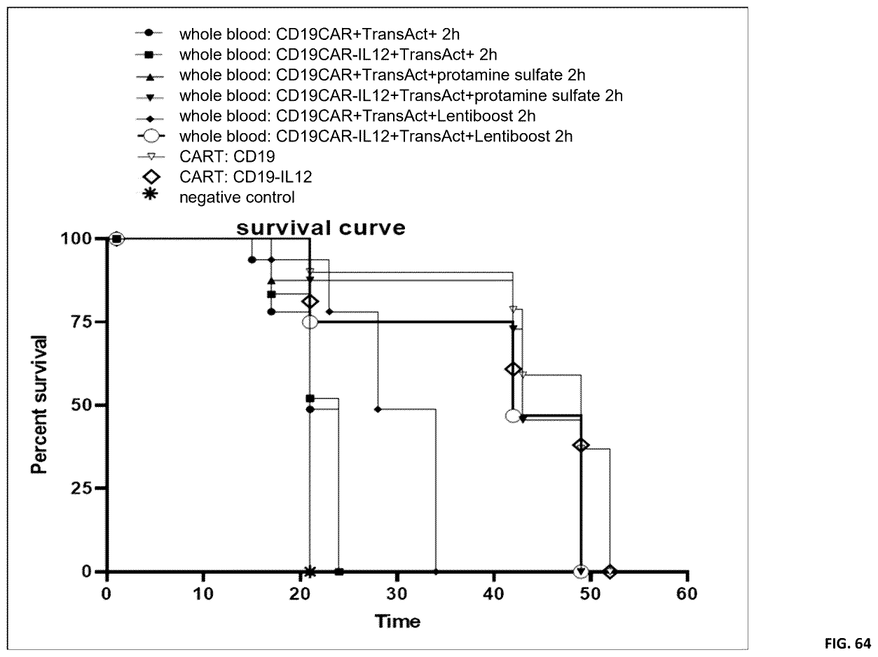

FIG. 64 shows the survival curve of mice that were infused with infected whole blood cells. Whole blood samples were obtained from volunteers. Some whole blood samples were infected with lentivirus vectors encoding CD19 CAR and IL-12 or CD19 CAR to obtain transfected whole blood cells. Some whole blood samples were purified to obtain CD3 positive cells, which were then infected with CD19 CAR and IL-12 or CD19 CAR alone. Each mouse was injected with 106 NALM6 tumor cells through the tail vein 7 days before reinfusion of infected cells. In the control group (tumor transplantation only), the survival days of mice without an infusion of CAR cells was 21 days. As shown in FIG. 64, PS and/or LentiBOOST™ enhanced survival times of mice that were infused with transfected whole blood cells, and IL-12 enhanced survival times of mice that were infused with transfected whole blood cells or T cells.

Thus, these in vivo results confirm that CAR-based treatment of cancer can be implemented by gene therapies through administering viral vectors encoding CAR directly to a subject having a form of cancer. As shown in the Examples, the gene therapies were enhanced by an increase in the transduction rate of the viral vectors and an increase of anti-tumor activities of the transduced CD3+ cells. In other words, the gene therapies were enhanced by administration of a T cell activator (e.g., CD3/CD28 agonists) and a transduction adjuvant (e.g., PS) and by co-expression of IL-12 in CD3+ cells.

All publications, patents, and patent applications cited in this specification are incorporated herein by reference in their entirety as if each individual publication, patent, or patent application were specifically and individually indicated to be incorporated by reference. While the foregoing has been described in terms of various embodiments, the skilled artisan will appreciate that various modifications, substitutions, omissions, and changes may be made without departing from the spirit thereof.

1. A chimeric antigen receptor (CAR) that binds Guanylyl Cyclase C (GCC), comprising amino acids 22 to 485 of SEQ ID NO: 9.

2. (canceled)

3. A polynucleotide encoding the CAR of claim 1.

4. An expression vector comprising the polynucleotide of claim 3.

5. The expression vector of claim 4, wherein the vector is a lentiviral vector.

6. A cell comprising the CAR of claim 1.

7. The cell of claim 6, wherein the cell is a T cell.

8. The cell of claim 7, wherein the cell is a human T cell.

9. A method of treating a subject having a cancer expressing GCC, the method comprising administering to the subject an effective amount of the cell of claim 6.

10. The method of claim 9, wherein the cancer is colorectal cancer.

11. A method of inhibiting the growth of a tumor expressing GCC in a subject, the method comprising administering to the subject an effective amount of the cell of claim 6.

12. The method of claim 11, wherein the tumor is a solid tumor selected from the group consisting of colorectal cancer, gastric cancer, esophageal cancer, and pancreatic cancer.

13. A method of inducing an immune response against a GCC-expressing cell in a subject, the method comprising administering to the subject the cell of claim 6.

14. A method of delivering a chimeric antigen receptor (CAR) to a T cell in vivo in a subject, the method comprising administering to the subject the expression vector of claim 4, wherein the expression vector transduces a T cell in the subject to express the CAR.

15. A cell comprising the expression vector of claim 4.

16. The cell of claim 6, further comprising a CAR that binds CD19.

17. A method of treating colorectal cancer in a subject, the method comprising administering to the subject an effective amount of the cell of claim 16.

18. A method of inhibiting the growth of a tumor expressing GCC in a subject, the method comprising administering to the subject an effective amount of the cell of claim 16.

19. The method of claim 18, wherein the tumor is colorectal cancer.

20. A method of inducing an immune response against a GCC-expressing cell in a subject, the method comprising administering to the subject the cell of claim 16.


SEQUENCE LISTING (9 sequences)

SEQ ID NO: 1 - protein, 21 aa, synthetic construct
  REGION 1..21: note = Synthesized
MALPVTALLLPLALLLHAARP

SEQ ID NO: 2 - protein, 69 aa, synthetic construct
  REGION 1..69: note = Synthesized
AKPTTTPAPRPPTPAPTIASQPLSLRPEACRPAAGGAVHTRGLDFACDIYIWAPLAGTCGVLLLSLVIT

SEQ ID NO: 3 - protein, 42 aa, synthetic construct
  REGION 1..42: note = Synthesized
KRGRKKLLYIFKQPFMRPVQTTQEEDGCSCRFPEEEEGGCEL

SEQ ID NO: 4 - protein, 112 aa, synthetic construct
  REGION 1..112: note = Synthesized
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5 - protein, 483 aa, synthetic construct
  REGION 1..483: note = Synthesized
MALPVTALLLPLALLLHAARPDIQMTQSPSSLSASVGDRVTITCRASQSVGSFLAWYQQKPGKAPKLLIYGASSRESGVPSRFSGSGSGTDFTLTISSLQPEDFATYYCQQTYHNPETFGQGTKVEIKGGGGSGGGGSGGGGSEVQLVESGGGLVQPGGSLRLSCAASGVSLPDYGVSWVRQAPGKGLEWVSVIWGSETTYYNSALKSRFTISRDNAKNSLYLQMNSLRAEDTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 6 - protein, 488 aa, synthetic construct
  REGION 1..488: note = Synthesized
MALPVTALLLPLALLLHAARPEVQLVQSGAEVKKPGQSLKISCKASGYSLTDNWIGWVRQKPGKGLEWMGIIYPGDSDTRYSPSFQGQVTISADKSINTAYLQWSSLKASDTAIYYCVGLDWNYNPLRYWGPGTLVTVSSGGGGSGGGGSGGGGSQSVLTQPPSVSAAPGQKVTISCSGSSSDIGSNYVSWYQQFPGTAPKLLIYDNNKRPSAIPDRFSGSKSGTSATLGITGLQTGDEADYYCGTWDSRLGIAVFGGGTQLTVLAKPTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - protein, 489 aa, synthetic construct
  REGION 1..489: note = Synthesized
MALPVTALLLPLALLLHAARPDIVMTQSPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPWTFGGGTKLEMKGGGGSGGGGSGGGGSQVQLQQSGAELARPGASVKMSCKASGNTFTSYTMRWVKQGPGQGLEWIGYINPSSGYTNYNKKFKDKATLTADKSSSTAYMQLSSLTSDDSAVYYCVFGNPRYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - protein, 15 aa, synthetic construct
  REGION 1..15: note = Synthesized
GGGGSGGGGSGGGGS

SEQ ID NO: 9 - protein, 485 aa, synthetic construct
  REGION 1..485: note = Synthesized
MALPVTALLLPLALLLHAARPEIVMTQSPATLSVSPGERATLSCRASQSVSRNLAWYQQKPGQAPRLLIYGASTRATGIPARFSGSGSGTEFTLTIGSLQSEDFAVYYCQQYKTWPRTFGQGTNVEIKGGGGSGGGGSGGGGSQVQLQQWGAGLLKPSETLSLTCAVFGGSFSGYYWSWIRQPPGKGLEWIGEINHRGNTNDNPSLKSRVTISVDTSKNQFALKLSSVTAADTAVYYCARERGYTYGNFDHWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR